(19) United States
(12) Patent Application Publication
(10) Pub. No.: US 2026/0346032 A1
(43) Pub. Date: Nov. 5, 2026

(54) VECTOR SWEEP WAVEFORMS FOR CARDIAC DEFIBRILLATORS

(71) Applicant: ZOLL Medical Corporation, Chelmsford, MA (US)

(72) Inventors: Nathan J. Berry Ann, Cranberry Township, PA (US); Richard T. Hanna, Bethel Park, PA (US); James T. Mourra, Pittsburgh, PA (US); Robyn I. Moyer, Verona, PA (US); Sean R. Pearson, Valencia, PA (US); Brad A. Radvansky, Cheswick, PA (US)

(73) Assignee: ZOLL Medical Corporation, Chelmsford, MA (US)

(21) Appl. No.: 19/565,744

(22) Filed: Mar. 13, 2026

Related U.S. Application Data
(60) Provisional application No. 63/772,003, filed on Mar. 14, 2025.

(51) Int. Cl. A61N 1/39

(52) U.S. Cl. CPC A61N 1/3918; A61N 1/3904

(57) ABSTRACT

A cardiac defibrillator configured to provide vector sweeping therapeutic shocks to a patient includes ECG electrodes, therapy electrodes configured to be positioned at predetermined locations on the patient, and a defibrillation controller. The defibrillation controller is configured to monitor ECG signals, determine whether the patient is experiencing a cardiac arrhythmia, and launch a treatment sequence in response to determining that the patient is experiencing a cardiac arrhythmia. The treatment sequence comprises alerting the patient that a defibrillation shock is imminent, upon receiving no indication from the patient that the patient is conscious, initiating delivery of vector sweeping defibrillation shocks, via the plurality of therapy electrodes, along a vector sweeping path around the patient's torso, and modulating one or more vector sweeping defibrillation waveforms of the vector sweeping defibrillation shocks to provide an approximately constant level of vector sweeping defibrillation current radially along the vector sweeping path.

### CROSS-REFERENCE TO RELATED APPLICATION

This nonprovisional application claims priority to U.S. Provisional Patent Application Ser. No. 63/772,003, filed on Mar. 14, 2025, titled “VECTOR SWEEP WAVEFORMS FOR CARDIAC DEFIBRILLATORS,” the entirety of which is hereby incorporated by reference.

### BACKGROUND

The present disclosure relates to a cardiac treatment system configured to treat cardiac arrhythmias occurring in various types of patients.

Heart disease, if left untreated, can lead to certain life-threatening arrhythmias. Both atrial and ventricular arrhythmias are common in patients with, for example, ischemic heart disease. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient in which various arrhythmias of the heart, such as ventricular fibrillation, ventricular tachycardia, pulseless electrical activity (PEA), and/or asystole (heart stops all electrical activity), result in the heart providing insufficient levels of blood flow to the brain and other vital organs for the support of life. It is generally useful to monitor heart disease patients to assess heart disease symptoms early and provide interventional therapies as soon as possible.

Cardiac treatment devices may provide defibrillation shocks to the patient if the patient starts experiencing an abnormal cardiac rhythm. Defibrillation shocks are programmed to effectively convert a patient's abnormal cardiac rhythm back to a normal cardiac rhythm. Defibrillation shocks may be delivered by an automated external defibrillator (AED). Otherwise, patients may be prescribed to wear cardiac treatment devices for extended periods of time, where the cardiac treatment device is configured to deliver defibrillation shocks upon determining that the patient is experiencing an abnormal cardiac rhythm.

### SUMMARY

In one or more examples, a cardiac defibrillator configured to provide vector sweeping therapeutic shocks to a patient is provided. The cardiac defibrillator includes a plurality of electrocardiogram (ECG) electrodes configured to sense cardiac electrical activity of an ambulatory patient, a plurality of therapy electrodes configured to be positioned at predetermined locations on the patient, and a defibrillation controller configured to be electrically connected to the plurality of ECG electrodes and the plurality of therapy electrodes. The defibrillation controller is configured to monitor ECG signals generated from the sensed cardiac electrical activity, determine whether the patient is experiencing a cardiac arrhythmia from the monitored ECG signals, and launch a treatment sequence in response to determining that the patient is experiencing a cardiac arrhythmia. The treatment sequence includes alerting the patient that a defibrillation shock is imminent, upon receiving no indication from the patient that the patient is conscious, initiating delivery of vector sweeping defibrillation shocks, via the plurality of therapy electrodes, along a vector sweeping path around the patient's torso, and modulating one or more vector sweeping defibrillation waveforms of the vector sweeping defibrillation shocks to provide an approximately constant level of vector sweeping defibrillation current radially along the vector sweeping path.

Implementations of the cardiac defibrillator configured to provide vector sweeping therapeutic shocks to a patient can include one or more of the following features. The vector sweeping path includes a 360 degree path around a vertical axis of the patient's torso. The approximately constant level of vector sweeping defibrillation current includes an average current between 3 and 75 Amps. The approximately constant level of vector sweeping defibrillation current includes an average current between 5 and 20 Amps. Modulating the one or more vector sweeping defibrillation waveforms of the vector sweeping defibrillation shocks includes applying pulse width modulation to provide the approximately constant level of vector sweeping defibrillation current radially along the vector sweeping path.

The approximate predetermined locations on the patient are spaced approximate 45 to 135 degrees radially from each other. The approximate predetermined locations on the patient are spaced approximately 60 to 120 degrees radially from each other. The approximate predetermined locations on the patient are spaced approximately 90 degrees radially from each other. The approximate predetermined locations on the patient are spaced approximately 60 degrees radially from each other. The plurality of therapy electrodes includes three or more therapy electrodes. The plurality of therapy electrodes includes four or more therapy electrodes.

The delivery of the vector sweeping defibrillation shock, via the plurality of therapy electrodes, is along a plurality of vectors across the patient's torso. The plurality of vectors are configured with fine vector angular resolution and smooth transitions between adjacent vectors. The plurality of vectors across the patient's torso are defined by a plurality of voltage waveforms administered to the patient by the plurality of therapy electrodes. The plurality of voltage waveforms include a plurality of half sinusoidal waveforms. The plurality of voltage waveforms include a plurality of synchronized multiple capacitor exponential decay curves.

Modulating the one or more vector sweeping defibrillation waveforms of the vector sweeping defibrillation shock includes modulating a therapy electrode voltage for each of the plurality of therapy electrodes according to an approximate sine wave. The approximate sine waves of the plurality of therapy electrodes are spaced radially even to each other. The approximate sine waves of the plurality of therapy electrodes are spaced radially even to each other at approximately 60 to 120 degree intervals. The approximate sine waves of the plurality of therapy electrodes are spaced radially even to each other at approximately 90 degree intervals. The approximate sine waves of the plurality of therapy electrodes are spaced radially even to each other at approximately 60 degree intervals. A peak of each approximate sine wave is between 400 V and 1250 V. A peak of each approximate sine wave is between 400 and 800 V.

The treatment sequence further includes generating an initial rising waveform. Generating the initial rising waveform includes generating the initial rising waveform before modulating the one or more vector sweeping defibrillation waveforms of the vector sweeping defibrillation shocks. Generating the initial rising waveform includes generating the initial rising waveform as part of modulating the one or more vector sweeping defibrillation waveforms of the vector sweeping defibrillation shocks.

Modulating the one or more vector sweeping defibrillation waveforms of the vector sweeping defibrillation shock includes modulating the one or more vector sweeping defibrillation waveforms to compensate for differences in tissue impedance along the vector sweeping path and thereby provide the approximately constant level of vector sweeping defibrillation current radially along the vector sweeping path. The defibrillation controller is further configured to take a plurality of tissue impedance measurements along the vector sweeping path. Modulating the one or more vector sweeping defibrillation waveforms to compensate for differences in tissue impedance along the vector sweeping path includes adjusting the one or more vector sweeping defibrillation waveforms according to the plurality of tissue impedance measurements. The defibrillation controller is further configured to receive one or more physical body measurements for the patient. Modulating the one or more vector sweeping defibrillation waveforms to compensate for differences in tissue impedance along the vector sweeping path includes adjusting the one or more vector sweeping defibrillation waveforms according to the one or more physical body measurements of the patient.

The defibrillation controller is further configured to detect a falloff signal at each of the plurality of therapy electrodes and determine, using the falloff signals, whether each of the therapy electrodes has a sufficient level of attachment to the patient. The defibrillation controller is further configured to adjust the delivery of the vector sweeping defibrillation shocks in response to detecting that one or more of the plurality of therapy electrodes lacks the sufficient level of attachment to the patient based on the falloff signals. The defibrillation controller is configured to adjust the delivery of the vector sweeping defibrillation shocks based on the predetermined locations on the patient associated with one or more of the plurality of therapy electrodes having the sufficient level of attachment to the patient. The defibrillation controller is configured to adjust the delivery of the vector sweeping defibrillation shocks by adjusting one or more voltage waveforms to be administered by the one or more of the plurality of therapy electrodes having the sufficient level of attachment to the patient. The defibrillation controller is configured to adjust the delivery of the vector sweeping defibrillation shocks by switching the delivery of the vector sweeping defibrillation shocks to multi-sequential defibrillation shocks to be administered by the one or more of the plurality of therapy electrodes having the sufficient level of attachment to the patient.

The cardiac defibrillator is configured as a wearable cardiac defibrillator. The plurality of ECG electrodes, the plurality of therapy electrodes, and the defibrillation controller are configured to be worn by the patient. The cardiac defibrillator further includes a garment configured to be worn about a torso of the patient. The plurality of ECG electrodes and the plurality of therapy electrodes are configured to be removably attached to the garment. At least one of the plurality of ECG electrodes or the plurality of therapy electrodes is permanently integrated into the garment. The garment is configured to support the defibrillation controller. The garment includes a belt configured to be worn about the torso of the patient. The garment further includes shoulder straps connected to the belt, the shoulder straps configured to be worn over shoulders of the patient. The cardiac defibrillator further includes one or more adhesive patches configured to be removably attached to a skin surface of the patient. The plurality of ECG electrodes are configured to be supported by at least one of the one or more adhesive patches. The plurality of therapy electrodes are configured to be supported by at least one of the one or more adhesive patches. The defibrillation controller is configured to be supported by at least one of the one or more adhesive patches.

The cardiac defibrillator is configured as an automatic external defibrillator. The cardiac defibrillator further includes one or more adhesive patches. The plurality of ECG electrodes and the plurality of therapy electrodes are configured to be supported by the one or more adhesive patches. The one or more adhesive patches include a single adhesive patch configured to support the plurality of ECG electrodes and the plurality of therapy electrodes.

In one or more examples, a method for providing vector sweeping therapeutic shocks to a patient using a wearable cardiac defibrillator is executed. The method includes monitoring electrocardiogram (ECG) signals generated from cardiac electrical activity of an ambulatory patient sensed by a plurality of ECG electrodes of the wearable cardiac defibrillator, determining whether the patient is experiencing a cardiac arrhythmia from the monitored ECG signals, and launching a treatment sequence in response to determining that the patient is experiencing a cardiac arrhythmia. The treatment sequence includes alerting the patient that a defibrillation shock is imminent, upon receiving no indication from the patient that the patient is conscious, initiating delivery of a vector sweeping defibrillation shock, via a plurality of therapy electrodes of the wearable cardiac defibrillator, along a vector sweeping path around the patient's torso, and modulating one or more vector sweeping defibrillation waveforms of the vector sweeping defibrillation shock to provide an approximately constant level of vector sweeping defibrillation current radially along the vector sweeping path.

Implementations of the method for providing vector sweeping therapeutic shocks to a patient using a wearable cardiac defibrillator can include one or more of the following features. The vector sweeping path includes a 360 degree path around a vertical axis of the patient's torso. The approximately constant level of vector sweeping defibrillation current includes an average current between 3 and 75 Amps. The approximately constant level of vector sweeping defibrillation current includes an average current between 5 and 20 Amps. Modulating the one or more vector sweeping defibrillation waveforms of the vector sweeping defibrillation shocks includes applying pulse width modulation to provide the approximately constant level of vector sweeping defibrillation current radially along the vector sweeping path.

The plurality of therapy electrodes includes three or more therapy electrodes. The plurality of therapy electrodes includes four or more therapy electrodes. The delivery of the vector sweeping defibrillation shock, via the plurality of therapy electrodes, is along a plurality of vectors across the patient's torso. The plurality of vectors are configured with fine vector angular resolution and smooth transitions between adjacent vectors. The plurality of vectors across the patient's torso are defined by a plurality of voltage waveforms administered to the patient by the plurality of therapy electrodes. The plurality of voltage waveforms include a plurality of half sinusoidal waveforms. The plurality of voltage waveforms include a plurality of synchronized multiple capacitor exponential decay curves.

Modulating the one or more vector sweeping defibrillation waveforms of the vector sweeping defibrillation shock includes modulating a therapy electrode voltage for each of the plurality of therapy electrodes according to an approximate sine wave. The approximate sine waves of the plurality of therapy electrodes are spaced radially even to each other. The approximate sine waves of the plurality of therapy electrodes are spaced radially even to each other at approximately 60 to 120 degree intervals. The approximate sine waves of the plurality of therapy electrodes are spaced radially even to each other at approximately 90 degree intervals. The approximate sine waves of the plurality of therapy electrodes are spaced radially even to each other at approximately 60 degree intervals. A peak of each approximate sine wave is between 400 V and 1250 V. A peak of each approximate sine wave is between 400 and 800 V.

The treatment sequence further includes generating an initial rising waveform. Generating the initial rising waveform includes generating the initial rising waveform before modulating the one or more vector sweeping defibrillation waveforms of the vector sweeping defibrillation shocks. Generating the initial rising waveform includes generating the initial rising waveform as part of modulating the one or more vector sweeping defibrillation waveforms of the vector sweeping defibrillation shocks.

Modulating the one or more vector sweeping defibrillation waveforms of the vector sweeping defibrillation shock includes modulating the one or more vector sweeping defibrillation waveforms to compensate for differences in tissue impedance along the vector sweeping path and thereby provide the approximately constant level of vector sweeping defibrillation current radially along the vector sweeping path. Taking a plurality of tissue impedance measurements along the vector sweeping path. Modulating the one or more vector sweeping defibrillation waveforms to compensate for differences in tissue impedance along the vector sweeping path includes adjusting the one or more vector sweeping defibrillation waveforms according to the plurality of tissue impedance measurements. Receiving one or more physical body measurements for the patient. Modulating the one or more vector sweeping defibrillation waveforms to compensate for differences in tissue impedance along the vector sweeping path includes adjusting the one or more vector sweeping defibrillation waveforms according to the one or more physical body measurements of the patient.

The method further includes detecting a falloff signal at each of the plurality of therapy electrodes and determining, using the falloff signals, whether each of the therapy electrodes has a sufficient level of attachment to the patient. The treatment sequence further includes adjusting the delivery of the vector sweeping defibrillation shocks in response to detecting that one or more of the plurality of therapy electrodes lacks the sufficient level of attachment to the patient based on the falloff signals. The defibrillation controller is configured to adjust the delivery of the vector sweeping defibrillation shocks by adjusting one or more voltage waveforms to be administered by the one or more of the plurality of therapy electrodes having the sufficient level of attachment to the patient. The defibrillation controller is configured to adjust the delivery of the vector sweeping defibrillation shocks by switching the delivery of the vector sweeping defibrillation shocks to multi-sequential defibrillation shocks to be administered by the one or more of the plurality of therapy electrodes having the sufficient level of attachment to the patient.

In one or more examples, a non-transitory computer-readable medium storing instructions that are configured to, when executed by one or more processors, cause the one or more processors to form a number of functions. The instructions cause the one or more processors to monitor electrocardiogram (ECG) signals generated from cardiac electrical activity of an ambulatory patient sensed by a plurality of ECG electrodes of a wearable cardiac defibrillator, determine whether the patient is experiencing a cardiac arrhythmia from the monitored ECG signals, and launch a treatment sequence in response to determining that the patient is experiencing a cardiac arrhythmia. The treatment sequence includes alerting the patient that a defibrillation shock is imminent, upon receiving no indication from the patient that the patient is conscious, initiating delivery of a vector sweeping defibrillation shock, via a plurality of therapy electrodes of the wearable cardiac defibrillator, along a vector sweeping path around the patient's torso, and modulating one or more vector sweeping defibrillation waveforms of the vector sweeping defibrillation shock to provide an approximately constant level of vector sweeping defibrillation current radially along the vector sweeping path.

Implementations of the non-transitory computer-readable medium can include one or more of the following features. The vector sweeping path includes a 360 degree path around a vertical axis of the patient's torso. The approximately constant level of vector sweeping defibrillation current includes an average current between 3 and 75 Amps. The approximately constant level of vector sweeping defibrillation current includes an average current between 5 and 20 Amps. Modulating the one or more vector sweeping defibrillation waveforms of the vector sweeping defibrillation shocks includes applying pulse width modulation to provide the approximately constant level of vector sweeping defibrillation current radially along the vector sweeping path. Implementations of the non-transitory computer-readable medium can also or alternatively include one or more features discussed above, such as with respect to the method for providing vector sweeping therapeutic shocks to a patient, discussed above.

In one or more examples, a cardiac defibrillator configured to provide vector sweeping therapeutic shocks to a patient is provided. The cardiac defibrillator includes a plurality of electrocardiogram (ECG) electrodes configured to sense cardiac electrical activity of an ambulatory patient, a plurality of therapy electrodes configured to be positioned at predetermined locations on the patient, and a defibrillation controller configured to be electrically connected to the plurality of ECG electrodes and the plurality of therapy electrodes. The defibrillation controller is configured to monitor ECG signals generated from the sensed cardiac electrical activity, determine whether the patient is experiencing a cardiac arrhythmia from the monitored ECG signals, and launch a treatment sequence in response to determining that the patient is experiencing a cardiac arrhythmia. The treatment sequence includes alerting the patient that a defibrillation shock is imminent, upon receiving no indication from the patient that the patient is conscious, delivering a first voltage waveform across the patient's torso using a first therapy electrodes from the plurality of therapy electrodes, and delivering a second voltage waveform across the patient's torso using a second therapy electrode from the plurality of therapy electrodes at least partially simultaneously with the first voltage waveform. The delivery of at least the first voltage waveform and the second voltage waveform cause an approximately constant level of vector sweeping defibrillation current to be delivered radially around the patient's torso.

Implementations of the cardiac defibrillator configured to provide vector sweeping therapeutic shocks can include one or more of the following features. The approximately constant level of vector sweeping defibrillation current includes an average current between 3 and 75 Amps. The approximately constant level of vector sweeping defibrillation current includes an average current between 5 and 20 Amps. The delivery of the at least the first voltage waveform and the second voltage waveform includes applying pulse width modulation to shape the at least the first waveform and the second waveform.

The approximate predetermined locations on the patient are spaced approximate 45 to 135 degrees radially from each other. The approximate predetermined locations on the patient are spaced approximately 60 to 120 degrees radially from each other. The approximate predetermined locations on the patient are spaced approximately 90 degrees radially from each other. The approximate predetermined locations on the patient are spaced approximately 60 degrees radially from each other. The plurality of therapy electrodes includes three or more therapy electrodes. The plurality of therapy electrodes includes four or more therapy electrodes.

The delivery of the at least the first voltage waveform and the second voltage waveform is along a plurality of vectors across the patient's torso. The plurality of vectors are configured with fine vector angular resolution and smooth transitions between adjacent vectors. The plurality of vectors across the patient's torso are defined by a plurality of voltage waveforms administered to the patient by the plurality of therapy electrodes. The plurality of voltage waveforms include a plurality of half sinusoidal waveforms. The plurality of voltage waveforms include a plurality of synchronized multiple capacitor exponential decay curves. The approximate predetermined locations on the patient are spaced approximately 90 degrees radially from each other. The approximate predetermined locations on the patient are spaced approximately 60 degrees radially from each other.

The first voltage waveform and the second voltage waveform each includes an approximate sine wave. The approximate sine waves of the first voltage waveform and the second voltage waveform are spaced radially from each other. The approximate sine waves of the first voltage waveform and the second voltage waveform are spaced radially even to each other at approximately a 60 to 120 degree interval. The approximate sine waves of the first voltage waveform and the second voltage waveform spaced radially even to each other at approximately a 90 degree interval. The approximate sine waves of the first voltage waveform and the second voltage waveform are spaced radially even to each other at approximately a 60 degree interval. A peak of each approximate sine wave is between 400 V and 1250 V. A peak of each approximate sine wave is between 400 and 800 V.

The treatment sequence further includes generating an initial rising waveform. Generating the initial rising waveform includes generating the initial rising waveform before delivering the first voltage waveform. Generating the initial rising waveform includes generating the initial rising waveform as part of delivering the first voltage waveform.

The treatment sequence further includes modulating at least one of the first voltage waveform or the second voltage waveform to compensate for differences in tissue impedance around the patient's body and thereby provide the approximately constant level of vector sweeping defibrillation current radially along the vector sweeping path. The defibrillation controller is further configured to take a plurality of tissue impedance measurements around the patient's body. Modulating the at least one of the first voltage waveform or the second voltage waveform to compensate for differences in tissue impedance around the patient's body includes adjusting the at least one of the first voltage waveform or the second voltage waveform according to the plurality of tissue impedance measurements. The defibrillation controller is further configured to receive one or more physical body measurements for the patient. Modulating the at least one of the first voltage waveform or the second voltage waveform to compensate for differences in tissue impedance around the patient's body includes adjusting the at least one of the first voltage waveform or the second voltage waveform according to the one or more physical body measurements of the patient.

The defibrillation controller is further configured to detect a falloff signal at each of the plurality of therapy electrodes and determine, using the falloff signals, whether each of the therapy electrodes has a sufficient level of attachment to the patient. The defibrillation controller is further configured to adjust the delivery of at least one of the first voltage waveform or the second voltage waveform in response to detecting that one or more of the plurality of therapy electrodes lacks the sufficient level of attachment to the patient based on the falloff signals. The defibrillation controller is configured to adjust the delivery of the at least one of the first voltage waveform or the second voltage waveform based on the predetermined locations on the patient associated with one or more of the plurality of therapy electrodes having the sufficient level of attachment to the patient. The defibrillation controller is configured to adjust the delivery of the at least one of the first voltage waveform or the second voltage waveform by adjusting one or more voltage waveforms to be administered by the one or more of the plurality of therapy electrodes having the sufficient level of attachment to the patient. The defibrillation controller is configured to adjust the delivery of the at least one of the first voltage waveform or the second voltage waveform by switching the delivery of the vector sweeping defibrillation shocks to multi-sequential defibrillation shocks to be administered by the one or more of the plurality of therapy electrodes having the sufficient level of attachment to the patient.

The cardiac defibrillator is configured as a wearable cardiac defibrillator. The plurality of ECG electrodes, the plurality of therapy electrodes, and the defibrillation controller are configured to be worn by the patient. A garment configured to be worn about a torso of the patient. The plurality of ECG electrodes and the plurality of therapy electrodes are configured to be removably attached to the garment. At least one of the plurality of ECG electrodes or the plurality of therapy electrodes is permanently integrated into the garment. The garment is configured to support the defibrillation controller. The garment includes a belt configured to be worn about the torso of the patient. The garment further includes shoulder straps connected to the belt, the shoulder straps configured to be worn over shoulders of the patient. The cardiac defibrillator includes one or more adhesive patches configured to be removably attached to a skin surface of the patient. The plurality of ECG electrodes are configured to be supported by at least one of the one or more adhesive patches. The plurality of therapy electrodes are configured to be supported by at least one of the one or more adhesive patches. The defibrillation controller is configured to be supported by at least one of the one or more adhesive patches.

The cardiac defibrillator is configured as an automatic external defibrillator. The cardiac defibrillator further includes one or more adhesive patches. The plurality of ECG electrodes and the plurality of therapy electrodes are configured to be supported by the one or more adhesive patches. The one or more adhesive patches include a single adhesive patch configured to support the plurality of ECG electrodes and the plurality of therapy electrodes.

In one or more examples, a cardiac defibrillator configured to provide vector sweeping therapeutic shocks to a patient is provided. A cardiac defibrillator includes a plurality of therapy electrodes configured to be positioned at predetermined locations on a patient experiencing a cardiac arrhythmia and a defibrillation controller configured to be electrically connected to the plurality of therapy electrodes. The defibrillation controller is configured to launch a treatment sequence to treat the patient experiencing a cardiac arrhythmia. The treatment sequence includes initiating delivery of a vector sweeping defibrillation shock, via the plurality of therapy electrodes, along a vector sweeping path around the patient's torso and modulating one or more vector sweeping defibrillation waveforms of the vector sweeping defibrillation shock to provide an approximately constant level of vector sweeping defibrillation current radially along the vector sweeping path.

Implementations of the cardiac defibrillator configured to provide vector sweeping therapeutic shocks to a patient can include one or more of the following features. The vector sweeping path includes a 360 degree path around a vertical axis of the patient's torso. The approximately constant level of vector sweeping defibrillation current includes an average current between 3 and 75 Amps. The approximately constant level of vector sweeping defibrillation current includes an average current between 5 and 20 Amps. Modulating the one or more vector sweeping defibrillation waveforms of the vector sweeping defibrillation shocks includes applying pulse width modulation to provide the approximately constant level of vector sweeping defibrillation current radially along the vector sweeping path. The cardiac defibrillator is configured as an automatic external defibrillator. The cardiac defibrillator further includes one or more adhesive patches. The plurality of therapy electrodes are configured to be supported by the one or more adhesive patches. The one or more adhesive patches include a single adhesive patch configured to support the plurality of therapy electrodes. Implementations of the cardiac defibrillator configured to provide vector sweeping therapeutic shocks to a patient may also or alternatively include one or more features discussed above, such as with respect to the cardiac defibrillator implementations discussed above. For instance, in examples, the cardiac defibrillator may include ECG electrodes.

In one or more examples, a cardiac defibrillator configured to provide vector sweeping therapeutic shocks to a patient is provided. The cardiac defibrillator includes a plurality of therapy electrodes configured to be positioned at predetermined locations on a patient, a defibrillation controller configured to be electrically connected to the plurality of therapy electrodes. The defibrillation controller is configured to deliver a first voltage waveform across the patient's torso using a first therapy electrode from the plurality of therapy electrodes and deliver a second voltage waveform across the patient's torso using a second therapy electrode from the plurality of therapy electrodes at least partially simultaneously with the first voltage waveform. The delivery of at least the first voltage waveform and the second voltage waveform cause an approximately constant level of vector sweeping defibrillation current to be delivered radially around the patient's torso.

Implementations of the cardiac defibrillator configured to provide vector sweeping therapeutic shocks to a patient can include one or more of the following features. The approximately constant level of vector sweeping defibrillation current includes an average current between 3 and 75 Amps. The approximately constant level of vector sweeping defibrillation current includes an average current between 5 and 20 Amps. The delivery of the at least the first voltage waveform and the second voltage waveform includes applying pulse width modulation to shape the at least the first waveform and the second waveform. The cardiac defibrillator is configured as an automatic external defibrillator. The cardiac defibrillator further includes one or more adhesive patches. The plurality of therapy electrodes are configured to be supported by the one or more adhesive patches. The one or more adhesive patches include a single adhesive patch configured to support the plurality of therapy electrodes. Implementations of the cardiac defibrillator configured to provide vector sweeping therapeutic shocks to a patient may also or alternatively include one or more features discussed above, such as with respect to the cardiac defibrillator implementations discussed above. For instance, in examples, the cardiac defibrillator may include ECG electrodes.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1A depicts an example wearable cardiac defibrillator.

FIG. 1B depicts an example electronic architecture for a wearable cardiac defibrillator.

FIG. 2 depicts an example therapy electrode placement for a cardiac defibrillator.

FIG. 3 depicts another example therapy electrode placement for a cardiac defibrillator.

FIG. 4 depicts another example therapy electrode placement for a cardiac defibrillator.

FIG. 5A depicts another example therapy electrode placement for a cardiac defibrillator.

FIG. 5B depicts another example therapy electrode placement for a cardiac defibrillator.

FIG. 5C depicts another example therapy electrode placement for a cardiac defibrillator.

FIG. 5D depicts another example therapy electrode placement for a cardiac defibrillator.

FIG. 5E depicts another example therapy electrode placement for a cardiac defibrillator.

FIG. 5F depicts another example therapy electrode placement for a cardiac defibrillator.

FIG. 5G depicts another example therapy electrode placement for a cardiac defibrillator.

FIG. 5H depicts another example therapy electrode placement for a cardiac defibrillator.

FIG. 6 depicts an example of vector sweeping defibrillation shocks that can be delivered by a cardiac defibrillator.

FIG. 7 depicts example graphs of defibrillation shocks using pulse width modulation.

FIG. 8 depicts other example graphs of defibrillation shocks using pulse width modulation.

FIG. 9 depicts example graphs of vector sweeping defibrillation shocks using pulse width modulation.

FIG. 10 depicts an example polar current plot for vector sweeping defibrillation shocks.

FIG. 11 depicts example modeled graphs of vector sweeping defibrillation shocks using pulse width modulation and an example modeled polar current plots for the vector sweeping defibrillation shocks.

FIG. 12 depicts another example graph of vector sweeping defibrillation shocks using pulse width modulation.

FIG. 13 depicts another example polar current plot for vector sweeping defibrillation shocks.

FIG. 14A depicts another example graph of defibrillation shocks using pulse width modulation.

FIG. 14B depicts another example graph of defibrillation shocks using pulse width modulation.

FIG. 14C depicts another example graph of defibrillation shocks using pulse width modulation.

FIG. 14D depicts another example of vector sweeping defibrillation shocks that can be delivered by a cardiac defibrillator.

FIG. 15A depicts an example circuit diagram for therapy electrodes.

FIGS. 15B-15E depict another example circuit diagram for therapy electrodes.

FIG. 16 depicts an example process flow for providing vector sweeping defibrillation shocks to a patient.

FIG. 17 depicts an example process flow for a treatment sequence.

FIG. 18 depicts another example process flow for a treatment sequence.

FIG. 19 depicts another example wearable cardiac defibrillator.

FIG. 20 depicts another example wearable cardiac defibrillator.

FIG. 21 depicts another example wearable cardiac defibrillator.

### DETAILED DESCRIPTION

This disclosure relates to electrotherapy waveforms for treating cardiac arrhythmias. Disclosed herein are techniques, electrode placements, and cardiac defibrillator architecture concepts for generating and delivering waveforms with features such as ascending ramps and quasi-sinusoidal waveforms, which are not provided by conventional cardiac defibrillator systems. Such waveform features, provided within waveforms known as vector sweep waveforms, allow for lower energy required to defibrillate and/or cardiovert, thus reducing risk of potential damage to cardiac tissue when compared to conventional waveforms (e.g., those provided by discharging a single capacitor or single capacitor bank using conventional waveshaping circuitry). Additional advantages are detailed below. In implementations herein, the cardiac defibrillator is configured to control one or more capacitors to discharge, for example, a patterned vector sweeping sequence of rapid current pulses to generate an averaged voltage and/or current with a predetermined waveform shape.

Cardiac devices and systems implementing the methods and techniques disclosed herein can be used in various settings to provide treatments, such as defibrillation, cardioversion, and/or pacing shocks in the event of life-threatening arrhythmias. Such cardiac devices may be, for example, defibrillators or cardioverter defibrillators. In examples, these defibrillators may include automated external defibrillators (AEDs) and/or wearable cardiac defibrillators. An AED may be used typically by a third party such as bystander or a healthcare professional to deliver treatment shocks to a patient who is experiencing a cardiac arrhythmia. The third party applies the AED to a patient suffering from a cardiac arrhythmia, and the AED then provides treatment shocks to the patient to restore a normal cardiac rhythm. The AED may include one or more adhesive patches configured to support a number of therapy electrodes and/or electrocardiogram (ECG) sensing electrodes. In examples, an AED may include both therapy electrodes and ECG electrodes configured to sense the patient's cardiac rhythm. Using the patient's cardiac rhythm, the AED may provide cardioversion therapy in time with the patient's cardiac rhythm, instruct the patient in how to provide chest compressions to the patient, and/or the like. In examples, an AED may include only therapy electrodes. In examples, the AED may include multiple adhesive patches, where each adhesive electrode supports one or more therapy and/or ECG electrodes. In examples, the AED may include one adhesive patch configured to support all of the therapy and/or ECG electrodes.

A wearable cardiac defibrillator is configured to be worn continuously by a patient over a prescribed period of time, as described in further detail below. The wearable cardiac defibrillator, upon detecting a cardiac arrhythmia is configured to alert the patient that the wearable cardiac defibrillator has determined the patient is experiencing the cardiac arrhythmia. The wearable cardiac defibrillator is configured to allow the patient to respond to the wearable cardiac defibrillator in the event of such an alert, such as by pressing one or more response buttons, to indicate that the patient is still conscious. If, instead, the patient does not respond within a predetermined time period, the wearable cardiac defibrillator is configured to proceed with delivering one or more treatment shocks to the patient.

In the case of defibrillation (or cardioversion) shocks, a cardiac defibrillator device as described herein is configured to deliver defibrillation shocks along various vectors through and/or around the patient's heart using various energies. In embodiments, the cardiac device is configured to deliver defibrillation shocks in a vector sweeping path around the patient's torso. For instance, the device is configured to deliver such vector sweeping defibrillation shocks as a successive series of therapeutic shocks in a predetermined rotational sequence of therapy vectors transversing a torso of a patient. As an illustration, a vector sweep defibrillation waveform may be generated by distributing electrodes about a cross-sectional plane of the patient's torso, such as roughly evenly about the cross-sectional plane, and then coordinating a sequence of shocks across these electrodes to deliver a rotating sequence of electric current vectors along the primary and/or intermediate axes of the electrode pairs. A primary axis may be formed, for example, directly between a pair of electrodes. By contrast, for example, an intermediate axis may be formed by delivering a voltage using uneven numbers of electrodes as the positive versus negative polarity of the vector.

In various examples, a cardiac device may deliver vector sweeping defibrillation shocks based on wave shaping techniques to control defibrillation waveforms of the defibrillation shocks. As an illustration, and as described in further detail below, the cardiac device is configured to employ pulse width modulation by varying the duty cycle of how one or more capacitors are discharged to create a predetermined voltage and/or current waveform applied to the patient. Such defibrillation waveforms created through pulse width modulation are delivered through various therapy electrodes positioned around the patient's torso to produce vector sweeping defibrillation shocks. In examples, the cardiac device uses pulse width modulation to control a desired defibrillation parameter for the defibrillation shocks, such as a desired current magnitude delivered around the patient's torso from multiple angles (e.g., radially around the patient's torso).

As such, this disclosure relates to cardiac defibrillators configured to provide vector sweeping therapeutic shocks to a patient. For example, in implementations, a cardiac defibrillator may include therapy electrodes configured to be positioned at predetermined locations on a patient who is experiencing a cardiac arrhythmia. The cardiac defibrillator may also include a defibrillation controller configured to be electrically connected to the therapy electrodes. The defibrillation controller is configured to launch a treatment sequence to treat the patient.

In implementations, during the treatment sequence, the defibrillation controller initiates delivery of a vector sweeping defibrillation shock, via the therapy electrodes, along a vector sweeping path around the patient's torso. Each electric current vector originates at the patient's skin on their torso and passes through their heart. To illustrate, the vector sweeping path may be defined by the placement of the therapy electrodes. For instance, the vector sweeping path may be on a transverse plane bisecting the patient's torso, where the center points of such therapy electrodes lie on the transverse plane. As another example, the vector sweeping path may be on a different plane bisecting the patient's torso, such as a plane along a diagonal to the transverse plane, where the center points of the therapy electrodes lie on the diagonal plane. As another example, the vector sweeping path may be along multiple planes, such as where the therapy electrodes are located on planes offset from each other.

In example implementations, the treatment sequence described herein also includes scenarios where the defibrillation controller is configured to use wave shaping techniques to control the vector sweeping waveforms. For example, the defibrillation controller is configured to modulate one or more vector sweeping waveforms of the vector sweeping defibrillation shock. As an illustration, the defibrillation controller may modulate one or more vector sweeping waveforms to provide an approximately constant level of vector sweeping defibrillation current radially along the vector sweeping path about the patient's torso. The defibrillation controller can be configured to ensure an approximately constant, predetermined level of vector sweeping defibrillation current, for example, by rapidly modulating the frequency, pulse width, and/or duty cycle of the one or more vector sweeping waveforms as the capacitor(s) supplying the defibrillation voltage discharge. For example, the defibrillation controller may modulate these aspects of the defibrillation waveforms on a scale of 10-100 microseconds, 10-100 milliseconds, 1-2 seconds, and/or the like. In various embodiments, the defibrillation controller may coordinate this modulation across three or more electrodes in order to shape the waveform along vectors that are intermediate to the primary electrode-pair axes. In implementations, the one or more vector sweeping waveforms that the defibrillation controller modulates may be or include one or more voltage waveforms and/or one or more current waveforms.

As another illustration, in implementations, the defibrillation controller delivers a series of waveforms across the patient's torso using two or more of the therapy electrodes during the treatment sequence. For instance, the defibrillation controller may deliver a first voltage waveform across the patient's torso using a first therapy electrode. The defibrillation controller may then deliver a second voltage waveform across the patient's torso using a second therapy electrode. In implementations, the defibrillation controller may deliver additional voltage waveforms using additional and/or the same therapy electrodes. The delivery of the voltage waveforms (e.g., at least the first voltage waveform and the second voltage waveform) causes an approximately constant level of vector sweeping defibrillation current to be delivered radially around the patient's torso.

In implementations, a cardiac defibrillator may also include electrocardiogram (ECG) electrodes configured to sense cardiac electrical activity of an ambulatory patient. For example, the cardiac defibrillator may sense cardiac electrical activity prior to, during, and/or after the treatment sequence. As such, the defibrillation controller may be configured to monitor ECG signals generated from the sensed cardiac electrical activity and determine whether the patient is experiencing a cardiac arrhythmia from the monitored ECG signals. The defibrillation controller thus may launch the treatment sequence in response to determining that the patient is experiencing a cardiac arrhythmia. In implementations, the treatment sequence further includes alerting the patient that a defibrillation shock is imminent. The defibrillation controller, as an illustration, may alert the patient that the defibrillation shock will be delivered within about 5 seconds to about 2 minutes from onset of the treatment sequence. For instance, the cardiac defibrillator may include a speaker configured to provide audio warnings, a light and/or a screen configured to provide visual warnings, a vibration box configured to provide haptic warnings, and/or the like, to alert the patient about the imminent shock. The treatment sequence therefore includes initiating delivery of the vector sweeping defibrillation shocks upon receiving no indication from the patient that the patient is conscious. To illustrate, the cardiac defibrillator may include one or more patient response buttons (e.g., positioned on the housing of the defibrillation controller, as a separate patient response unit, and/or the like) that the patient can press to indicate to the cardiac defibrillator that the patient is still conscious. If the patient presses the one or more patient response buttons, the defibrillation controller may delay or cancel the vector sweeping defibrillation shocks. If, instead, the patient does not press the one or more patient response buttons within a predetermined amount of time, the defibrillation controller determines that no indication from the patient that the patient is conscious has been received and proceeds with the vector sweeping defibrillation shocks, as outlined above. In examples, the cardiac defibrillator may provide the patient with 5 seconds, 10 seconds, 30 seconds, 45 seconds, another user-defined duration, and/or the like before the cardiac defibrillator proceeds with the treatment sequence and delivers the vector sweeping defibrillation shocks.

In one example use case, a cardiologist may prescribe that a patient at risk for developing a life-threatening arrhythmia use a wearable cardiac defibrillator until the patient can receive an implantable defibrillator. The wearable cardiac defibrillator includes a garment that the patient wears around their torso, an electrode belt that is assembled into the garment, and a defibrillation controller configured to be connected to the electrode belt. The electrode belt includes ECG sensing electrodes and therapy electrodes that are configured to be disposed at predetermined locations on the patient when the patient is wearing the wearable cardiac defibrillator. For instance, these predetermined locations may be spaced equally apart radially around the patient's torso. The defibrillation controller is configured to sense when the patient is experiencing a cardiac arrhythmia, such as by using ECG signals generated from cardiac electrical activity sensed by the ECG electrodes. In response to determining that the patient is experiencing a cardiac arrhythmia, the defibrillation controller launches a treatment sequence. As part of the treatment sequences, the defibrillation controller provides therapy in the form of vector sweeping defibrillation shocks across a vector sweeping path around the patient's torso. For example, each of the therapy electrodes may include one or more capacitors configured to be charged when the defibrillation controller detects a cardiac arrhythmia. The defibrillation controller may then control circuitry of the therapy electrodes to administer or release stored energy in therapeutic shocks to the patient. As the one or more capacitors discharge, the defibrillation controller may use pulse width modulation to control the shape of one or more defibrillation waveforms. To illustrate, the defibrillation controller may use pulse width modulation to create an approximately constant current delivered around the patient's torso. In implementations, the constant current may be, for example, a constant average current delivered around the patient's torso.

The defibrillation controller is also configured to, prior to delivering the vector sweeping defibrillation shocks, deliver one or more alarms to the patient to alert the patient of the impending therapy. The defibrillation controller may deliver the one or more alarms via user interfaces located on the defibrillation controller itself, located on a signal processing node also included on the electrode belt, located on a separate patient interface pod, and/or the like. Alarms may include auditory alarms (e.g., a siren, a voice instruction, and/or the like), visual alarms (e.g., flashing lights, a warning screen, and/or the like), tactile alarms (e.g., vibrations delivered by a tactile alarm mechanism, and/or the like), and/or so on. After observing the one or more alarms, the patient may indicate to the defibrillation controller that the patient is still conscious by pressing one or more response buttons. Similar to the user interfaces configured to deliver alarms, the one or more response buttons may be located on the defibrillation controller, the signal processing node, a separate patient interface pod, and/or the like. The cardiac controller may delay or abort the therapy delivery in response to the patient pressing the one or more response buttons.

In another example use case, a cardiologist may prescribe a wearable cardiac defibrillator for a patient at risk of developing life-threatening arrhythmias but who is not physically well enough to receive an implantable defibrillator. The wearable cardiac defibrillator may include adhesive therapy components configured to be removably adhesively attached to the patient's torso. In examples, the adhesive therapy components may include ECG electrodes configured to sense cardiac electrical activity of the patient and therapy electrodes configured to deliver therapeutic shocks. The adhesive therapy components are configured to be electrically coupled to a defibrillation controller. The patient may wear the defibrillation controller or the cardiac controller may also be adhesively attached to the patient. As an example, the patient may wear the defibrillation controller in a carrying case, for instance, configured to attach to the patient's belt, be worn using a shoulder strap, and/or the like. As another example, the patient may wear the defibrillation controller mounted on a garment that the patient wears around their torso. The defibrillation controller determines when the patient is experiencing a cardiac arrhythmia condition and, in response, controls the delivery of vector sweeping therapeutic shocks. Part of controlling the delivery of the vector sweeping therapeutic shocks may include modulating one or more defibrillation waveforms to create desired voltage and/or current waveforms that the defibrillation controller delivers in a vector sweeping pattern using the therapy electrodes.

In another example use case, a patient undergoing a cardiac arrhythmia may be treated with an AED. A third party, such as a bystander or first responder, may connect an AED to a patient undergoing a cardiac arrhythmia. For example, the third party can attach adhesive patches to the patient, where each of the adhesive patches supports one or more therapy electrodes and/or one or more ECG electrodes. Additionally, the adhesive patches are configured to be attached to the patient such that the therapy electrodes are positioned at predetermined locations on the patient. The adhesive patches are electrically connected to a defibrillation controller, which can then deliver vector sweeping defibrillation shocks via the therapy electrodes. In implementations, the defibrillation controller may sense the patient's cardiac rhythm using the ECG electrodes and synchronize the vector sweeping defibrillation shocks with the patient's cardiac rhythm. The defibrillation controller is also configured to modulate one or more defibrillation waveforms of the vector sweeping defibrillation shocks to provide, for instance, a desired current around the patient's torso. As an illustration, the defibrillation controller may be configured to modulate the duty cycle of the vector sweeping defibrillation shocks to produce an approximately constant level of vector sweeping defibrillation current radially around the patient's torso. For example, the approximately constant level of defibrillation current may be an average constant level of defibrillation current around the patient's torso.

The cardiac defibrillators described herein may provide advantages over prior art systems. Delivering precisely timed shocks, which may be overlapping, across three or more therapy electrodes can produce the vector sweeping effect described earlier. This vector sweeping effect can engage a circular sequence of electric field vectors through the patient's heart, thereby reducing the total energy required for defibrillation compared to a single shock. Additionally, by using pulse width modulation to control defibrillation waveforms, the cardiac defibrillators can deliver defibrillation shocks without abrupt jumps between different vectors. Instead, for example, the cardiac defibrillators can create a smooth sweep of vectors covering the plane of the electrodes at a high resolution. By removing discrete jumps in defibrillation vectors through defibrillation waveforms shaped using pulse width modulation, as described in further detail below, the cardiac defibrillators may increase the probability of the electric field vector being in parallel to the anatomical orientation of the cardiomyocytes in the patient's heart. Defibrillating according to vectors parallel with a given cardiomyocytes is known to be the optimal orientation for depolarizing the cardiomyocyte with minimal field strength. In implementations, as also described below, cardiac defibrillators may generate vectors sweeping defibrillation shocks with an initial rising waveform. These rising waveforms may further reduce the abrupt jumps in the vector sweeping defibrillation shocks, thereby also reducing tissue damage and increasing the probability of cardiomyocyte depolarization.

Additionally, the cardiomyocytes in the human heart are oriented non-uniformly, and in various cases, 75-90% of ventricular cardiomyocytes must be depolarized simultaneously for effective defibrillation. Furthermore, the biophysical mechanism of defibrillation is believed to be a threshold function. In particular, current greater than a “rheobase,” or a minimum threshold of electric current required to trigger an action potential in the cardiomyocytes, must be applied for a “chronaxie” duration, or a minimum amount time needed for the current to stimulate the cardiomyocytes. Therefore, the minimum current of the defibrillation shocks must be greater than the rheobase for the chronaxie. By using pulse width modulation as described in further detail below as part of delivering vector sweeping defibrillation shocks, a cardiac defibrillator may shape defibrillation waveforms to better generate 1) a desired average of current 2) across a large number of vectors. Accordingly, such cardiac defibrillators can more easily deliver the rheobase of current in the orientation of the ventricular cardiomyocytes, thus lowering the amount of unnecessary current applied to the patient, increasing the effectiveness of the defibrillation shocks, and reducing the damage to cardiac tissues during defibrillation.

Moreover, the increased effectiveness of such vector sweeping defibrillation schemes can mean that less overall energy is needed during defibrillation. Lower defibrillation energy means reduced stress on the heart during defibrillation and reduced chances of skin burns. The lower defibrillation energy may also be associated with improved return of spontaneous circulation, cerebral performance, and myocardial function. Additionally, in turn, less required energy may allow the number and/or size of capacitors needed for such cardiac defibrillators, which would mean lighter, smaller, and less expensive cardiac defibrillators that are able to provide the same or better level of care. For wearable cardiac defibrillators, the decreased weight and size may also make such wearable cardiac defibrillators more comfortable for patients to wear during their daily lives, leading to increased patient compliance with prescriptions to continuously use the wearable cardiac defibrillators. Alternatively, or additionally, such cardiac defibrillators may need to be charged less frequently due to the decreased energy requirements.

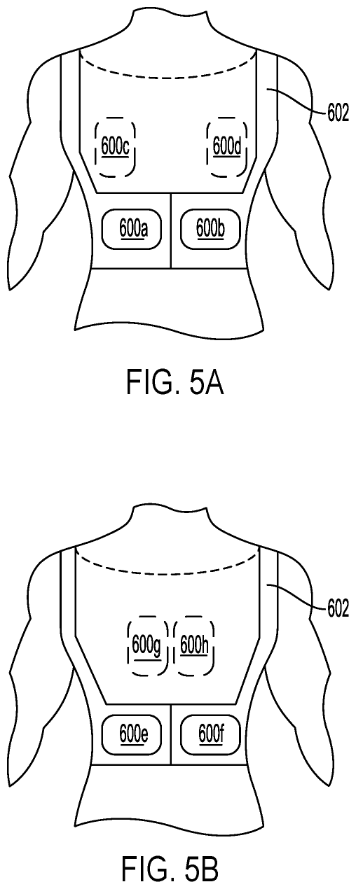

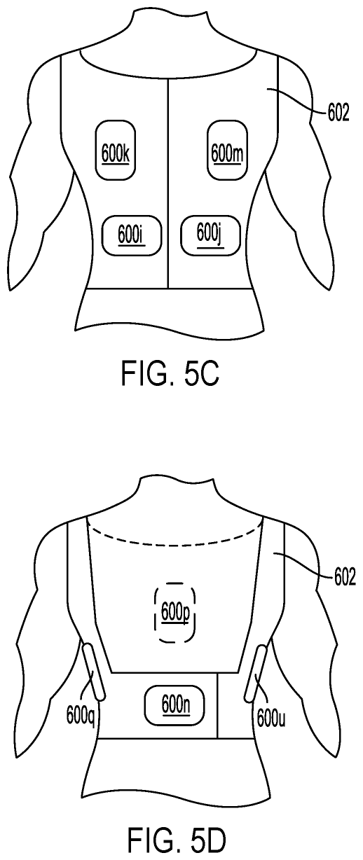

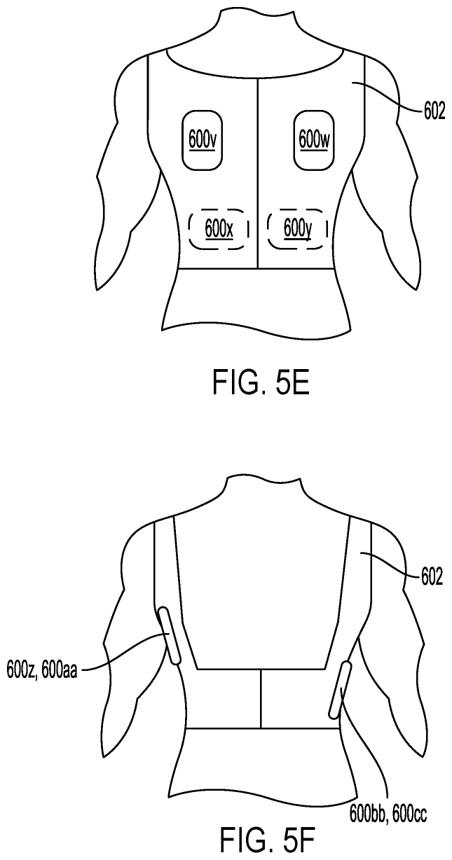

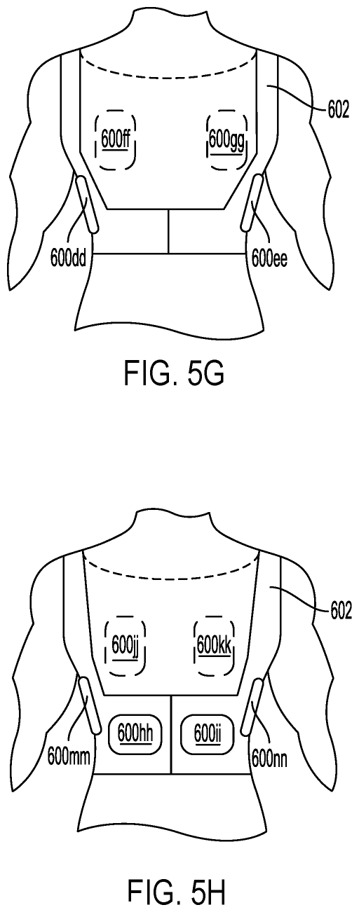

Using pulse width modulation to shape defibrillation waveforms may be particularly beneficial with vector sweeping defibrillation. In implementations, when sweeping defibrillation vectors around the patient's torso and through the patient's heart, if the cardiac defibrillator uses the same energy for all shocks, some of the vectors may deliver far more current than the patient needs. For example, if the patient receives a defibrillation shock from two pairs of electrodes positioned at approximately 90-degree angles around the patient's body (e.g., as shown in FIG. 5D), the cardiac defibrillator may activate the first pair to create a vector directly between the two electrodes of the first pair, both pairs simultaneously to create a diagonal vector between the two pairs of electrodes, and the second pair to create a vector directly between the electrodes of the second pair. If the cardiac defibrillator uses the same energy for each activation, the diagonal vector will deliver twice the amount of current as the two vectors directly between the electrode pairs. However, by using pulse width modulation to shape the defibrillation waveforms, the cardiac defibrillator may deliver defibrillation shocks using the same amount of current for each vector.

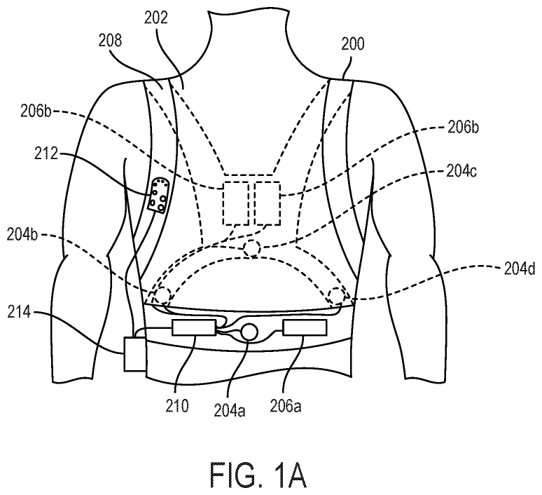

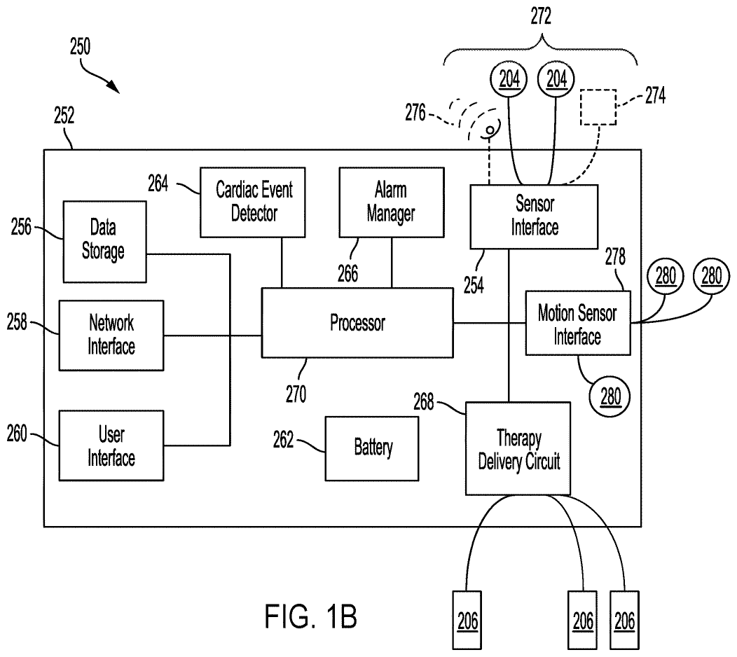

As described above, cardiac devices and systems implementing the vector sweep methods and techniques discussed herein may include, for example, various types of cardiac defibrillators. For example, cardiac defibrillators may include wearable cardiac defibrillators configured to detect when a patient is experiencing a cardiac arrhythmia and provide a treatment to the patient and/or AEDs configured to be used by a third party (e.g., a bystander, a medical professional, etc.) to provide a treatment to a patient experiencing a cardiac arrhythmia. FIG. 1A illustrates an example of a wearable cardiac defibrillator 200, according to implementations disclosed herein. As shown in FIG. 1A, the wearable cardiac defibrillator 200 is external and wearable by a patient 202 around the patient's torso. Such a wearable cardiac defibrillator 200, for instance, can be capable and designed for moving with the patient 202 as the patient 202 goes about their daily routine. In examples, the wearable cardiac defibrillator 200 may be configured to be bodily-attached to the patient 202. To illustrate, the patient 202 may don the wearable cardiac defibrillator 200 about the patient's torso and secure the wearable cardiac defibrillator 200 in place through a closure. In example scenarios, the wearable cardiac defibrillator 200 can be worn nearly continuously or substantially continuously for an extended period of time, such as a week, two weeks, a month, two months, three months, six months, etc. During the period of time in which the wearable cardiac defibrillator 200 is worn by the patient 202, the wearable cardiac defibrillator 200 can be configured to continuously or substantially continuously monitor the vital signs of the patient 202 and can be configured to, upon determination that treatment is required, deliver one or more therapeutic electrical pulses to the patient 202. For example, such therapeutic shocks can be pacing, defibrillation, cardioversion, and/or transcutaneous electrical nerve stimulation (TENS) pulses. In embodiments, the wearable cardiac defibrillator 200 may be a wearable cardioverter defibrillator and configured to synchronize therapeutic shocks with the patient's cardiac cycles.

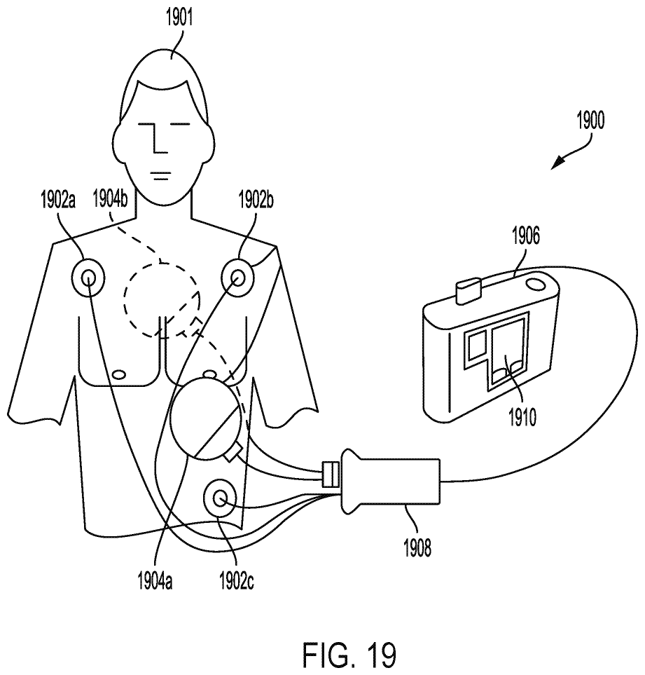

As noted above, regardless of the extended period of wear, the use of the wearable cardiac defibrillator 200, as well as other embodiments of wearable cardiac defibrillators discussed below with reference to FIGS. 19-21, can include continuous or nearly continuous wear by the patient as described above. For example, the continuous use can include continuous wear or attachment of the wearable cardiac defibrillator to the patient during both periods of monitoring and periods when the wearable cardiac defibrillator may not be monitoring the patient but is otherwise still worn by or otherwise attached to the patient. The wearable cardiac defibrillator can be configured to continuously monitor the patient for cardiac-related information (e.g., ECG information, including arrhythmia information, cardiovibrations, and/or the like) and/or non-cardiac information (e.g., blood oxygen, the patient's temperature, glucose levels, tissue fluid levels, lung vibrations, and/or the like). The wearable cardiac defibrillator can carry out its monitoring in periodic or aperiodic time intervals or times. For example, the monitoring during intervals or times can be triggered by a user action or another event. As an illustration, the wearable cardiac defibrillator may continuously monitor the patient while the wearable cardiac defibrillator is turned on and detects at least a minimum level of electrode attachment to the patient.

As noted above, the wearable cardiac defibrillator is continuously used, except for sporadic periods during which the use temporarily ceases. Examples of temporary cessation include while the patient bathes, while the patient is refit with a new and/or a different garment, while adhesive patches are being replaced, while the battery is charged/changed, while the garment is laundered, and/or the like. Such substantially or nearly continuous use as described herein may nonetheless be considered continuous use. For example, the wearable cardiac defibrillator can be configured to be worn by a patient for as many as 24 hours a day. As another example, the patient can remove the wearable cardiac defibrillator for a short portion of the day (e.g., for half an hour to bathe).

As shown in FIG. 1A, the wearable cardiac defibrillator 200 can include one or more ECG electrodes 204a-204d (collectively referred to herein as ECG electrodes 204) configured to contact the patient's skin and sense cardiac electrical activity of the patient 202, one or more therapy electrodes 206a and 206b (collectively referred to herein as therapy electrodes 206) configured to deliver therapeutic shocks to the heart of the patient 202, and a defibrillation controller 214 configured to be electrically or operably connected to the ECG electrodes 204 and the therapy electrodes 206. In implementations, the one or more ECG electrodes 204, the one or more therapy electrodes 206, and the defibrillation controller 214 are configured to be worn by the patient 202. For example, as shown, the wearable cardiac defibrillator 200 can also include a garment 208 configured to be worn about the torso of the patient 202, where the garment 208 is configured to support at least the ECG electrodes 204 and the therapy electrodes 206. In implementations, the garment 208 may include a belt configured to be worn about the patient's torso. In implementations, the garment 208 may further include shoulder straps connected to the belt, where the shoulder straps are configured to be worn over the patient's shoulders. To illustrate, as shown, the garment 208 may be configured in a vest-like configuration for wear over the patient's upper torso.

In implementations, the wearable cardiac defibrillator 200 can include additional elements. Additional elements may include, for instance, a signal processing node 210, a patient interface pod 212, additional sensors or detectors, or any combination of these, as further illustrated in FIG. 1A. Examples of additional sensors or detectors include one or more motion detectors configured to generate motion data indicative of physical activity being performed by the patient 202, wear state sensors configured to detect a wear state of the wearable cardiac defibrillator 200 (e.g., whether the patient 202 is wearing the wearable cardiac defibrillator 200 or not), vibrational or bioacoustics sensors configured to generate cardiovibrational or bioacoustics signals indicative of cardiovibrational or bioacoustics markers for the heart of the patient 202, respiration sensors configured to generate respiration signals indicative of respiration activity of the patient 202, thoracic fluid sensors configured to generate thoracic fluid signals indicative of a thoracic fluid level of the patient 202, and/or the like.

In implementations, some or all of the ECG electrodes 204, the therapy electrodes 206, the signal processing node 210, the patient interface pod 212, additional sensors or detectors, and/or the like may be configured to be assembled onto the garment 208. To illustrate, some or all of the ECG electrodes 204, the therapy electrodes 206, the signal processing node 210, the patient interface pod 212, additional sensors or detectors, and/or the like may be configured to be removably attached to the garment 208. In examples, at least some of the components of the wearable cardiac defibrillator 200 can be configured to be removably attached, mounted, or affixed on the garment 208, such as by mating hooks, hook-and-loop fabric strips, receptacles (e.g., pockets), snaps (e.g., plastic or metal snaps), and/or the like. As an illustration, the ECG electrodes 204 may be removably attached to the garment 208 by hook-and-loop fabric strips on the ECG electrodes 204 and the garment 208. As another illustration, the therapy electrodes 206 may be removably attached to the garment 208 by being inserted into receptacles (e.g., pockets with conductive mesh) on the garment 208. In implementations, at least some of the components of the wearable cardiac defibrillator 200 (e.g., at least one of the ECG electrodes 204, therapy electrodes 206, signal processing node 210, patient interface pod 212, additional sensors or detectors, and/or the like) can be permanently integrated into the garment 208. For instance, at least some of the components of the wearable cardiac defibrillator 200 may be sewn into the garment 208 and/or adhesively secured to the garment 208 with a permanent adhesive.

In examples, at least some of the components may be connected to each other through external cables, through internal or sewn-in connections (e.g., wires woven into the fabric of the garment 208, wires woven between layers of the garment 208, and/or the like), through conductive fabric of the garment 208, and/or the like. Component configurations other than those shown in FIG. 1A are also possible. For example, the ECG electrodes 204 may be configured to be attached at other positions about the body of the patient 202 and/or may include additional or fewer ECG electrodes 204. As another example, the therapy electrodes 206 may be configured to be attached at other positions about the body of the patient 202 and/or may include additional or fewer electrodes 206, such as discussed in further detail below with respect to FIGS. 5A-5H.

As noted above, the defibrillation controller 214 can be configured to be electrically or operatively coupled to the ECG electrodes 204 and the therapy electrodes 206. In implementations, the defibrillation controller 214 may be configured to be directly coupled to at least some of the ECG electrodes 204 and/or therapy electrodes 206. In implementations, the defibrillation controller 214 may be configured to be indirectly coupled to at least some of the ECG electrodes 204 and/or therapy electrodes 206, such as through another component of the wearable cardiac defibrillator 200. For example, and as shown in FIG. 1A, the defibrillation controller 214 can be indirectly coupled to at least some of the ECG electrodes 204 and/or the therapy electrodes 206 through the signal processing node 210. In implementations, the garment 208 may be configured to support the defibrillation controller 214. To illustrate, the defibrillation controller 214 may also be configured to be assembled into the garment 208. For example, the entire defibrillation controller 214 may be configured to be inserted into and/or attached to a receptacle of the garment 208, such as a pocket. As another example, the functions of the defibrillation controller 214 may be dispersed among multiple defibrillation controller units (e.g., a cardiac arrhythmia monitoring unit, a therapy delivery unit, a communications unit, an alarm unit, etc.). The multiple defibrillation controller units, for instance, may include individual units for each of some or all of the components shown in the electronic architecture of FIG. 1B. To illustrate, these multiple defibrillation controller units may then be inserted into and/or attached to receptacles of the garment 208. As another illustration, these multiple defibrillation controller units may be permanently integrated into garment 208. For example, defibrillation controller units can be sewn and/or permanently adhered into the garment 208.

As noted above, the ECG electrodes 204 can be configured to sense cardiac electrical activity of the patient 202. Example ECG electrodes 204 may include a metal electrode with an oxide coating, such as tantalum pentoxide electrodes. For instance, by design, the ECG electrodes 204 can include skin-contacting electrode surfaces that may be deemed polarizable or non-polarizable depending on a variety of factors, including the metals and/or coatings used in constructing the electrode surface. All such electrodes can be used with the principles, techniques, devices, and systems described herein. For instance, the electrode surfaces can be based on stainless steel, noble metals such as platinum, or Ag—AgCl. In implementations, the ECG electrodes 204 can be used with an electrolytic gel dispersed between the electrode surface and the patient's skin. In implementations, the ECG electrodes 204 can be dry electrodes that do not need an electrolytic material. As an example, such a dry electrode can be based on tantalum metal, such as by having a tantalum pentoxide coating as is described above. Such dry electrodes can be more comfortable for long-term monitoring applications. Based on the cardiac electrical activity of the patient 202 sensed by the ECG electrodes 204, the ECG electrodes 204 can generate ECG signals for the patient 202, which may be further amplified, digitized, filtered, and/or the like as described in further detail below.

In implementations, the ECG electrodes 204 can include additional components such as accelerometers or other types of motion detectors, cardiac acoustic signal detecting devices (e.g., cardiovibrational sensors), and/or other measuring devices for recording other types of parameters for the patient 202. For example, the ECG electrodes 204 can also be configured to detect other patient physiological parameters and/or acoustic signals, such as tissue fluid levels, heart vibrations, lung vibrations, respiration vibrations, patient movement, etc. Similarly, in implementations, the therapy electrodes 206 can additionally or alternatively be configured to include sensors configured to detect cardiac electrical activity of the patient 202 (e.g., therapy electrodes 206 can be combined with ECG electrodes 204). In examples, the therapy electrodes 206 can additionally or alternatively be configured to include sensors configured to detect other physiological parameters or signals from the patient 202, such as the physiological parameters or signals described above.

The signal processing node 210 can, in various examples, include a signal processor configured to amplify, filter, and/or digitize ECG signals prior to transmitting the ECG signals to the defibrillation controller 214. As an example, the signal processing node 210 may be configured to reduce and/or remove noise in the ECG signals received from the ECG electrodes 204. As another example, the signal processing node 210 may be configured to digitize the ECG signals received from the ECG electrodes 204, such as through an analog-to-digital converter. In implementations, the ECG electrodes 204 may instead include circuitry to amplify, filter, and/or digitize signals prior to transmitting the signals to the defibrillation controller 214. For example, the ECG electrodes 204 may digitize ECG signals, and the signal processing node 210 may reduce and/or remove noise using ground signals from a ground electrode. In implementations, the defibrillation controller 214 instead may perform at least some of the amplifying, filtering, and/or digitizing of the ECG signals.

In implementations, the wearable cardiac defibrillator 200 may include a driven ground electrode. The driven ground electrode may be implemented as one of the ECG electrodes 204, one of the therapy electrodes 206, and/or as a separate electrode provided elsewhere on the wearable cardiac defibrillator 200. The driven ground electrode may be used to reduce the effects of noise and/or detect if an electrode (e.g., one of the ECG electrodes 204) has fallen off from the patient's skin surface or otherwise become disconnected from the rest of the wearable cardiac defibrillator 200. For example, in implementations, the signal processing node 210 or another component of the wearable cardiac defibrillator 200 (e.g., the defibrillation controller 214) may inject a signal having a higher frequency than the frequencies normally observed in an ECG waveform via the driven ground electrode. The ECG electrodes 204 can detect the high-frequency driven ground signals. Detection of the high-frequency driven ground signals (e.g., falloff signals) by a given ECG electrode 204 indicates that the given ECG electrode 204 is still in contact with the patient's skin. Failing to detect the high-frequency driven ground signals may be an indication that the respective ECG electrode 204 has fallen off of the patient's skin. The defibrillation controller 214 can assign lower confidence levels to signals derived from ECG electrodes 204 that the defibrillation controller 214 has determined have fallen off.

Although the signal processing node 210 is shown as attached to the garment 208 at patient's front in FIG. 1A, in examples the signal processing node 210 may be attached to the garment 208 at the small of the patient's back. In such implementations, because the signal processing node 210 is located against the small of the patient's back where the patient 202 may be sensitive to feeling movement, the signal processing node 210 can be configured to include one or more vibration motors to provide tactile notifications to the patient 202. For instance, the signal processing node 210 can receive one or more signals from the defibrillation controller 214 and provide a tactile alert to the patient 202, as described in further detail below.

In implementations, the defibrillation controller 214 is configured to monitor the ECG signals generated from the cardiac electrical activity sensed by the ECG electrodes 204 and determine when the patient 202 is experiencing a cardiac arrhythmia. The therapy electrodes 206 are configured to deliver one or more electrical therapeutic shocks to the patient 202, such as one or more therapeutic cardioversion/defibrillation shocks to the body of the patient 202, when the defibrillation controller 214 determines that treatment is warranted. For instance, treatment can include vector sweeping defibrillation shocks, as described in further detail below. Example therapy electrodes 206 can include conductive metal electrodes such as stainless-steel electrodes. In implementations, a cardiac defibrillator may include three or more therapy electrodes, as shown in FIG. 1A. In implementations, a cardiac defibrillator may include four or more therapy electrodes. In implementations, the therapy electrodes 206 include one or more conductive gel deployment devices configured to deliver conductive gel between the metal electrode and the patient's skin prior to delivery of a therapeutic shock. The functionality of the defibrillation controller 214 is described in further detail below with respect to FIG. 1B.

In implementations, the signal processing node 210 may be configured to control at least part of the delivery of the one or more therapeutic shocks. As an example, the signal processing node 210 may receive a signal from the defibrillation controller 214 initiating therapy delivery. The signal processing node 210 may send a signal to the therapy electrodes 206 to activate the deployment of conductive electrolytic gel at the therapy electrodes 206 (e.g., from an integrated or removable gel pack on each therapy electrode 206). The signal processing node 210 may then receive one or more therapeutic charges from the defibrillation controller 214 and convey the one or more therapeutic charges to the therapy electrodes 206 for delivery of the one or more therapeutic shocks to the patient 202.

In implementations, the defibrillation controller 214 is also configured to warn the patient 202 prior to the delivery of a therapy. For example, the defibrillation controller 214 may be configured to transmit one or more warnings to the patient 202 via one or more output devices integrated into or connected to the defibrillation controller 214, the signal processing node 210, and/or the patient interface pod 212. As an illustration, in implementations, the patient interface pod 212 can be secured to a shoulder strap of the garment 208, such as via a hook-and-loop fastener, a plastic or metal snap connector, a clip, a buckle, etc. Thus assembled, the patient interface pod 212 may then issue warnings to the patient 202. In implementations, a warning may be auditory. For example, a warning may be or include a siren alarm, a voice instruction indicating that the patient 202 is going to be shocked, and/or the like. In implementations, a warning may be visual. To illustrate, a warning may be or include flashing lights on the defibrillation controller 214 and/or the patient interface pod 212, a warning display on a screen of the defibrillation controller 214, and/or the like. In implementations, a warning may be haptic. As an illustration, a warning may be or include a tactile, buzzing alarm generated by a haptic device of the signal processing node 210, and/or the like. In implementations, a warning may include a combination of auditory, visual, and/or haptic alarms.

As an example of the foregoing, the defibrillation controller 214 may deliver alarms warning the patient 202 of an impending therapeutic shock via speakers on the defibrillation controller 214 and/or the signal processing node 210 (e.g., where the alarm includes a siren that increases in volume over time, verbal warnings that the patient 202 is going to receive a therapeutic shock, verbal instructions informing the patient 202 to avoid a therapeutic shock by pressing the one or more response buttons, as outlined below, and/or the like). As another example, the defibrillation controller 214 may deliver alarms via a screen or other lights of the defibrillation controller 214 or patient interface pod 212 (e.g., by displaying a warning on the screen of the defibrillation controller 214, by lighting up LEDs on the defibrillation controller 214, and/or the like). As another example, the defibrillation controller 214 may deliver alarms by causing the signal processing node 210 to vibrate against the patient's back. In implementations, alarms may include combinations of some or all of these examples. In implementations, alarms may escalate over time, such as by including louder sounds, higher frequencies, more frequent warnings, stronger vibrations, and/or the like.

If the patient 202 is still conscious, the patient 202 may be able to delay or stop the delivery of the therapeutic shock. In implementations, the patient 202 may press one or more response buttons to indicate to the defibrillation controller 214 that the patient 202 is still conscious. Such response buttons may be provided, for example, on the defibrillation controller 214 and/or on the patient interface pod 212. In response to the patient 202 pushing the one or more response buttons, the defibrillation controller 214 may delay or stop the delivery of the therapeutic shock. For example, the defibrillation controller 214 may include response buttons provided on opposite sides of the defibrillation controller 214 that the patient 202 must push simultaneously to delay or cancel the therapeutic shock. In such examples, the wearable cardiac defibrillator 200 may not include a patient interface pod 212. As another example, the patient interface pod 212 may include one or more response buttons that the patient 202 must push to delay or cancel the therapeutic shock. In implementations, the patient 202 may need to press a response button on the patient interface pod 212 and the defibrillation controller 214 simultaneously to delay or cancel the therapeutic shock. In implementations, the patient interface pod 212 may be implemented as a wireless response button unit, for example, implemented as a watch or wristband that the patient 202 wears along with the garment 208. As another illustration, the wearable cardiac defibrillator 200 may not include a patient interface pod. Instead, a patient user device, such as a smartphone, may serve as the response button unit. For example, the defibrillation controller 214 may communicate with the smartphone (e.g., via Bluetooth®, via cellular networks, etc.) and cause the smartphone to display a button, a link, or so on that the patient 202 must press to delay or cancel the therapeutic shock. In implementations, the wearable cardiac defibrillator 200 may include combinations of the above types of response buttons.

FIG. 1B illustrates a sample component-level view of a defibrillation controller 250, which is an example of the defibrillation controller 214 of FIG. 1A. As shown in FIG. 1B, the defibrillation controller 250 may include a housing 252 configured to house a number of electronic parts. These electronic parts may include any or all of a sensor interface 254, a data storage 256, a network interface 258, a user interface 260, at least one battery 262 (e.g., positioned within a battery chamber configured for such a purpose), a cardiac event detector 264, an alarm manager 266, a therapy delivery circuit 268, and/or at least one processor 270. In implementations, the processor 270 includes one or more processors (or one or more processor cores) that are each configured to perform a series of instructions that result in the manipulation of data and/or the control of the operation of the other components of the defibrillation controller 250. In implementations, when executing a specific process (e.g., monitoring sensed surface electrical signals of the patient 202, determining when to apply vector sweeping therapeutic shocks to the patient 202, and/or the like), the processor 270 can be configured to make specific logic-based determinations based on input data received. The processor 270 may be further configured to provide one or more outputs that can be used to control or otherwise inform subsequent processing to be carried out by the processor 270 and/or other processors or circuitry to which the processor 270 is communicably coupled. Thus, the processor 270 reacts to a specific input stimulus in a specific way and generates a corresponding output based on that input stimulus. In example cases, the processor 270 can proceed through a sequence of logical transitions in which various internal register states and/or other bit cell states internal or external to the processor 270 may be set to logic high or logic low.

As referred to herein, the processor 270 can be configured to execute a function where software is stored in a non-transitory computer-readable data store (e.g., the data storage 256) coupled to the processor 270, the software being configured to cause the processor 270 to proceed through a sequence of various logic decisions that result in the function being executed. The various components that are described herein as being executable by the processor 270 can be implemented in various forms of specialized hardware, software, or a combination thereof. For example, the processor 270 can be a digital signal processor (DSP) such as a 24-bit DSP processor. As another example, the processor 270 can be a multi-core processor, e.g., having two or more processing cores. As another example, the processor 270 can be an Advanced RISC Machine (ARM) processor, such as a 32 bit ARM processor. The processor 270 can execute an embedded operating system and further execute services provided by the operating system, where these services can be used for file system manipulation, display and audio generation, basic networking, firewalling, data encryption, communications, and/or the like.

The data storage 256 can include one or more of non-transitory computer-readable media, such as flash memory, solid state memory, magnetic memory, optical memory, cache memory, combinations thereof, and others. The data storage 256 can be configured to store executable instructions and data used for operation of the defibrillation controller 250. In implementations, as described above, the data storage 256 can include sequences of executable instructions that, when executed, are configured to cause the processor 270 to perform one or more functions. Additionally, the data storage 256 can be configured to store information such as digitized ECG signals of the patient 202.

In examples, the network interface 258 can facilitate the communication of information between the defibrillation controller 250 and one or more devices or entities over a communications network. For example, the network interface 258 can be configured to communicate with a remote server or other similar computing device. Using the network interface 258, the wearable cardiac defibrillator 200 may transmit, for example, ECG signals, other physiological signals, indications of abnormal cardiac events, etc., to the remote server. In implementations, the network interface 258 can include communications circuitry for transmitting data in accordance with a Bluetooth® wireless standard for exchanging such data over short distances to an intermediary device or devices (e.g., a base station, “hotspot” device, smartphone, tablet, portable computing device, and/or other device in proximity with the wearable cardiac defibrillator 200). The intermediary device(s) may in turn communicate the data to the remote server over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high speed wireless communication. In some implementations, the intermediary device(s) may communicate with the remote server over a Wi Fi communications link based on the IEEE 802.11 standard. In implementations, the network interface 258 may be configured to instead communicate directly with the remote server without the use of intermediary device(s). In such implementations, the network interface 258 may use any of the communications links and/or protocols provided above to communicate directly with the remote server.

The sensor interface 254 can include physiological signal circuitry that is coupled to one or more sensors 272 configured to be externally applied to the patient 202. As shown, the sensors 272 may be coupled to the defibrillation controller 250 via a wired or wireless connection. The sensors 272 may include, for example, the ECG electrodes 204 configured to sense surface electrical signals indicative of ECG activity from the skin surface of the patient 202, as well as one or more non-ECG sensors, such as a cardiovibration sensor 274 and/or a tissue fluid monitor 276. Other examples of external sensors 272 may include a respiration sensor, a bioacoustics sensor, a blood pressure sensor, a temperature sensor, a pressure sensor, a humidity sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and/or the like.

As an illustration, the sensor interface 254 may receive ECG signals generated by the ECG electrodes 204 along one or more ECG channels, where each ECG channel includes a pair of ECG electrodes 204. In implementations, and as discussed above, the sensor interface 254 may receive ECG signals that are preprocessed. For example, the ECG electrodes 204 and/or the signal processing node 210 may digitize, filter, remove noise, and/or the like from the ECG signals before sending the ECG signals to the sensor interface 254. In implementations, the sensor interface 254 may perform at least some of the processing of the ECG signals. The sensor interface 254, for instance, may digitize, filter, remove noise, and/or the like. In examples, the sensor interface 254 may use driven ground signals from a driven ground electrode to determine whether the ECG electrodes 204 are still sufficiently in contact with the patient's skin, as discussed above.

In implementations, the sensor interface 254 and/or the processor 270 may be configured to provide digitized ECG signals of the patient 202 based on electrical signals sensed by the ECG electrodes 204. In this sense, the sensor interface 254 and/or the processor 270 may be considered an ECG digitizing circuit. The digitized ECG signals of the patient 202 may be stored in the data storage 256. In implementations, the signal processing node 210 may instead perform the digitizing of the ECG signals, as discussed above. In implementations, the ECG electrodes 204 themselves may instead digitize the ECG signals, as further discussed above.

As previously described, the sensor interface 254 and the motion sensor interface 278 can be coupled to any one or combination of external sensors to receive patient data indicative of patient parameters. Once data from the sensors has been received by the sensor interface 254 and/or the motion sensor interface 278, the data can be directed by the processor 270 to an appropriate component within the defibrillation controller 250. For example, ECG signals collected by the ECG electrodes 204 may arrive at the sensor interface 254, and the sensor interface 254 may transmit the ECG signals to the processor 270, which, in turn, relays the patient's ECG data to the cardiac event detector 264. The sensor data can also be stored in the data storage 256 and/or transmitted to a remote server via the network interface 258.

In implementations, the cardiac event detector 264 can be configured to monitor the patient's ECG signal for an occurrence of a cardiac event such as an arrhythmia or other similar cardiac event. The cardiac event detector 264 can be configured to operate in concert with the processor 270 to execute one or more methods that process received ECG signals from, for example, the ECG electrodes 204 and determine the likelihood that the patient 202 is experiencing a cardiac event, such as a cardiac arrhythmia. The cardiac event detector 264 can be implemented using hardware or a combination of hardware and software. For instance, in examples, the cardiac event detector 264 can be implemented as a software component that is stored within the data storage 256 and executed by the processor 270. In this example, the instructions included in the cardiac event detector 264 can cause the processor 270 to perform one or more methods for analyzing a received ECG signal to determine whether an adverse cardiac event is occurring, such as a cardiac arrhythmia. In other examples, the cardiac event detector 264 can be an application-specific integrated circuit (ASIC) that is coupled to the processor 270 and configured to monitor ECG signals for adverse cardiac event occurrences. Thus, examples of the cardiac event detector 264 are not limited to a particular hardware or software implementation.

In implementations, the cardiac event detector 264 may determine that the patient is experiencing an arrhythmia using a combination of heart rate and/or cardiac cycle morphology. For example, the cardiac event detector 264 may determine the patient's heart rate from ECG signals (e.g., generated by the sensor interface 254). As an illustration, the cardiac event detector 264 may use a QRS detector to identify R peaks within the ECG signals and calculate the patient's heart rate based on the time period between adjacent R peaks. As another illustration, the cardiac event detector 264 may perform a fast Fourier transform (FFT) on the ECG signals, with the FFT decomposing the analog ECG waveform into its frequency components. The cardiac event detector 264 may then analyze the output of the FFT to determine the strongest frequency component indicative of heart rate. Once the cardiac event detector 264 has determined the patient's heart rate, the cardiac event detector 264 may then identify whether the patient's heart rate transgresses one or more arrhythmia thresholds. To illustrate, the cardiac event detector 264 may determine if the patient's heart rate is below a threshold for ventricular tachycardia, at or above the threshold for ventricular tachycardia but below a threshold for ventricular fibrillation, or at or above the threshold for ventricular fibrillation.

As another example, the cardiac event detector 264 may alternately or additionally determine the patient's current vectorcardiogram from their ECG signals. For instance, the ECG electrodes 204 may be positioned around the patient's torso when the patient is wearing the wearable cardiac defibrillator 200 to form orthogonal leads. To illustrate, the orthogonal leads may include leads that are front-to-back and side-to-side at the level of the patient's xiphoid process. The cardiac event detector 264 may determine a direction and magnitude of the electrical forces in the patient's heart and plot them to form a vectorcardiogram. For example, the cardiac event detector 264 may plot the direction and magnitude of the patient's electrical forces on an x-y or an x-y-z graph. The cardiac event detector 264 may then determine whether the patient's current vectorcardiogram matches a baseline vectorcardiogram. To illustrate, the cardiac event detector 264 may take a baseline vectorcardiogram for the patient 202 during a setup period and/or periodically during the patient's use of the wearable cardiac defibrillator 200. For instance, the cardiac event detector 264 may take a new baseline vectorcardiogram for the patient 202 weekly at a predetermined time, after the patient 202 is delivered a therapeutic shock, and/or the like. The cardiac event detector 264 may then compare the patient's current vectorcardiogram to the patient's baseline vectorcardiogram to determine if the two morphologies match with a predetermined degree of accuracy. If the patient's current vectorcardiogram does not match their baseline vectorcardiogram, this failure to match may serve as evidence that the patient 202 is experiencing a cardiac arrhythmia. If the patient's current vectorcardiogram does match their baseline vectorcardiogram with the predetermined degree of accuracy, this match may serve as evidence that the patient 202 is not experiencing a cardiac arrhythmia.

In implementations, the cardiac event detector 264 may use a combination of heart rate and/or cardiac cycle morphology to determine whether the patient is experiencing a cardiac arrhythmia. For example, the cardiac event detector 264 may determine whether the patient's current vectorcardiogram matches a baseline vectorcardiogram in response to the patient's heart rate transgressing an arrhythmia threshold. The cardiac event detector 264 may then output an indication that the patient 202 is experiencing an arrhythmia if the patient's current vectorcardiogram does not match their baseline vectorcardiogram with a predetermined degree of accuracy. In implementations, the cardiac event detector 264 may use additional inputs and/or measures to determine whether the patient is experiencing an arrhythmia. For instance, the cardiac event detector 264 may use cardiovibrational signals from the cardiovibration sensor 274 to determine the patient's heart rate (e.g., based on the presence of S1 and S2 cardiovibrational biomarkers corresponding to the closing of the patient's heart valves). As another example, the cardiac event detector 264 may use the cardiovibrational signals to detect the whether the patient is exhibiting S3 or S4 cardiovibrational biomarkers, which may indicate heart disease.

In implementations, the user interface 260 may include one or more physical interface devices, such as input devices, output devices, and combination input/output devices, as well as a software stack configured to drive operation of the devices. These user interface elements may render visual, audio, and/or tactile content. Thus, the user interface 260 may receive inputs and/or provide outputs, thereby enabling a user to interact with the defibrillation controller 250. Examples of user interfaces 260 include speakers, display screens, touch screens, tactile motors, and/the like.

The defibrillation controller 250 can also include at least one battery 262 configured to provide power to one or more electronic parts integrated in the defibrillation controller 250. The battery 262 can include a rechargeable multi-cell battery pack. In one example implementation, the battery 262 can include three or more cells (e.g., 2200 mA lithium-ion cells) that provide electrical power to the other device components within the defibrillation controller 250. For example, the battery 262 can provide its power output in a range of between a 20 mA to 1000 mA (e.g., 40 mA) output and can support 24 hours, 48 hours, 72 hours, or more of runtime between charges. In certain implementations, the battery capacity, runtime, and type (e.g., lithium-ion, nickel-cadmium, or nickel-metal hydride) can be changed to best fit the specific application of the defibrillation controller 250.

Additionally, the wearable cardiac defibrillator 200 is configured to provide therapeutic shocks to the patient 202 upon the cardiac event detector 264 detecting that the patient 202 is experiencing a cardiac arrhythmia. The therapy delivery circuit 268 can be coupled to the therapy electrodes 206 that are configured to provide therapy to the patient 202. For example, the therapy delivery circuit 268 can include, or be operably connected to, circuitry components that are configured to generate and provide an electrical therapeutic shock. The circuitry components can include, for example, resistors, capacitors, relays and/or switches, electrical bridges such as an H bridge (e.g., including a plurality of insulated gate bipolar transistors or IGBTs), voltage and/or current measuring components, and other similar circuitry components. The circuitry components are arranged and connected such that the circuitry components work in concert with the therapy delivery circuit 268 and under the control of one or more processors (e.g., processor 270) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and/or the like. In implementations, defibrillation or cardioversion pulses can be used to treat ventricular tachycardia and/or ventricular fibrillation.

In implementations, the therapy delivery circuit 268 includes a first high-voltage circuit connecting a first pair of the therapy electrodes 206 (e.g., the therapy electrode 206a and one of the therapy electrodes 206b) and a second high-voltage circuit connecting a second pair of the therapy electrodes 206 (e.g., the therapy electrode 206a and the other of the therapy electrodes 206b). Using the high-voltage circuits, the therapy delivery circuit 268 can deliver a first therapeutic shock, such as a first biphasic therapeutic pulse, via the first high-voltage circuit and a second therapeutic shock, such as a second biphasic therapeutic pulse, via the second high-voltage circuit. In implementations, the second high-voltage circuit is configured to be electrically isolated from the first high-voltage circuit. In implementations, the therapy delivery circuit 268 includes a capacitor configured to be selectively connected to the first high-voltage circuit and/or the second high-voltage circuit. As such, the first high-voltage circuit may be powered by the capacitor when the capacitor is selectively connected to the first high-voltage circuit, and the second high-voltage circuit may be powered by the capacitor when the capacitor is selectively connected to the second high-voltage circuit. In implementations, the therapy delivery circuit 268 includes a first capacitor electrically connected to the first high-voltage circuit and a second capacitor electrically connected to the second high-voltage circuit.

The capacitors can include a parallel-connected capacitor bank consisting of a plurality of capacitors (e.g., two, three, four, or more capacitors). In some examples, the capacitors can include a single film or electrolytic capacitor as a series connected device, including a bank of the same capacitors. These capacitors can be switched into a series connection during discharge for a defibrillation pulse. For example, four capacitors of approximately 140 μF or larger can be used. As another example, a single capacitor of approximately 162.5 μF or four capacitors of approximately 650 μF each can be used. The capacitors can have a 1600 VDC or higher rating for a single capacitor, or a surge rating between approximately 350 to 500 VDC for paralleled capacitors, and can be charged in approximately 15 to 30 seconds from a battery pack.

For example, each defibrillation pulse can deliver between 60 J to 180 J of energy. The therapy delivery circuit 268 can be configured to perform the switching and pulse delivery operations, e.g., under control of the processor 270. As the energy is delivered to the patient 202, the amount of energy being delivered can be tracked. For example, the amount of energy can be kept to a predetermined constant value even as the pulse waveform is dynamically controlled based on factors such as the patient's body impedance while the pulse is being delivered. In certain examples, the therapy delivery circuit 268 can be configured to deliver a set of cardioversion pulses to correct, for example, an improperly beating heart. When compared to defibrillation as described above, cardioversion often includes a less powerful shock that is delivered at a certain frequency to mimic a heart's normal rhythm.

The alarm manager 266 can be implemented using hardware or a combination of hardware and software. For instance, in examples, the alarm manager 266 can be implemented as a software component that is stored within the data storage 256 and executed by the processor 270. In these examples, the instructions included in the alarm manager 266 can cause the processor 270 to configure alarm profiles and notify intended recipients using the alarm profiles. In other examples, the alarm manager 266 can be an application-specific integrated circuit (ASIC) that is coupled to the processor 270 and configured to manage alarm profiles and notify intended recipients using alarms specified within the alarm profiles. Thus, examples of the alarm manager 266 are not limited to a particular hardware or software implementation.

In implementations, the alarm manager 266 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, and/or monitoring personnel), as well as computer systems (e.g., monitoring systems and/or emergency response systems). For example, in response to the cardiac event detector 264 described above determining that the patient 202 is experiencing an arrhythmia, the therapy delivery circuit 268 is configured to deliver a cardioversion/defibrillation shock to the patient 202 via the therapy electrodes 206. However, first the alarm manager 266 may issue an alarm (e.g., via the user interface(s) 260, via the patient interface pod 212, and/or via the signal processing node 210, as described above) that the patient 202 is about to experience a defibrillating shock. The alarm may include visual, auditory, tactile, and/or other types of alerts. In implementations, the alerts may increase in intensity over time. Examples of increasing intensity include increasing in pitch, increasing in volume, increasing in frequency, switching from a tactile alert to an auditory alert, and/or the like.

Additionally, in implementations, the alerts may inform the patient 202 that the patient 202 can abort the delivery of the defibrillating shock by interacting with the user interface 260 or another component of the wearable cardiac defibrillator 200. For instance, the patient 202 may be able to press a user response button or user response buttons of the user interface 260, after which the alarm manager 266 will cease issuing an alert and the therapy delivery circuit 268 will no longer prepare to deliver or will delay delivering the therapeutic shock. As described above, in implementations, the patient 202 may additionally or alternatively be able to interact with one or more user interface buttons disposed elsewhere on the wearable cardiac defibrillator 200 to abort or delay the therapeutic shock. To illustrate, the patient may be able to interact with the patient interface pod 212, such as by pressing a user response button or user response buttons on the patient interface pod 212.

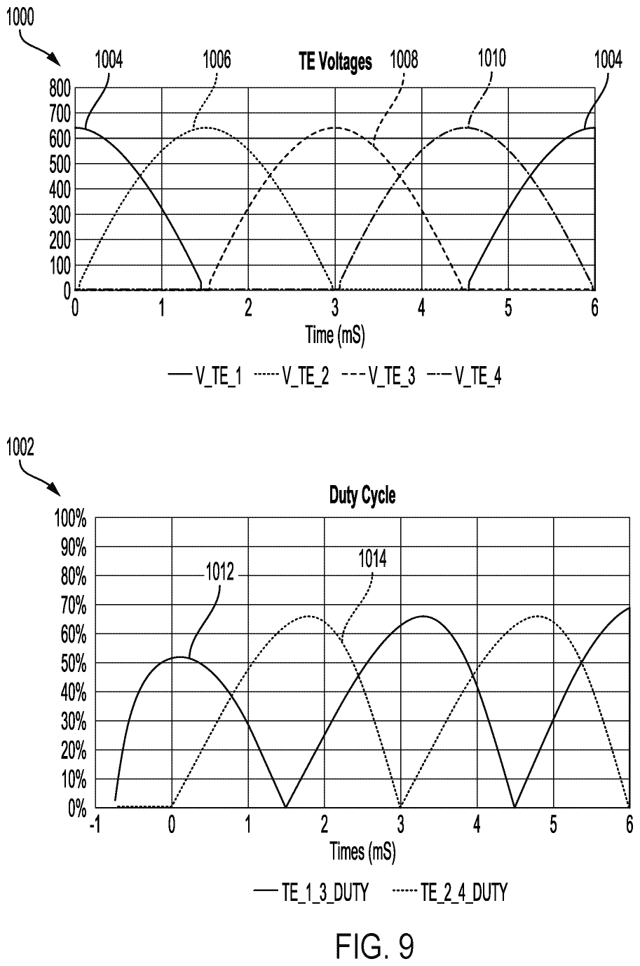

In implementations, a cardiac defibrillator, such as the wearable cardiac defibrillator 200 discussed above or the cardiac defibrillators discussed below with respect to FIGS. 19-21, can be configured to provide vector sweeping therapeutic shocks to an ambulatory patient. With vector sweeping therapeutic shocks, the cardiac defibrillator delivers energy in a sequence with defined timing, where a goal is to “sweep” the current direction through the patient's body through the heart at varying angles along a vector sweeping path. In examples, the vector sweep traverses a transverse plane bisecting the patient's torso (e.g., through a center point of the patient's heart). In examples, the vector sweep traverses an angled plane at a predetermined angle (e.g., between 1 to 75 degrees) to a transverse plane bisecting the patient's torso (e.g., through a center point of the patient's heart). In implementations, a cardiac defibrillator may deliver a vector sweeping defibrillation shock via the therapy electrodes along a number of vectors on a transverse or angled plane across the patient's torso. As described in further detail below, e.g., with reference to FIGS. 9-13 below, in various implementations the vectors are configured with fine vector angular resolution and smooth transitions between adjacent vectors. For example, the vectors originating from points across the patient's torso may be defined by a number of voltages administered to that patient by the therapy electrodes. Illustrations of such voltage waveforms may include a number of half sinusoidal waveforms and/or a number of synchronized multiple-capacitor exponential decay curves.

In implementations, a vector sweeping path includes a 360-degree path on a transverse or angled plane centered about a vertical axis through the patient's torso. Accordingly, therapy electrodes can be configured to be positioned at predetermined anatomical locations on the patient to provide the vector sweeping therapeutic shocks. In implementations, the vector sweeping path may be pre-defined, as a matter of design constraint or choice. For example, the vector sweeping path may be defined by the physical placement of the therapy electrodes at the predetermined anatomical locations and the vectors that can be formed between the therapy electrodes.

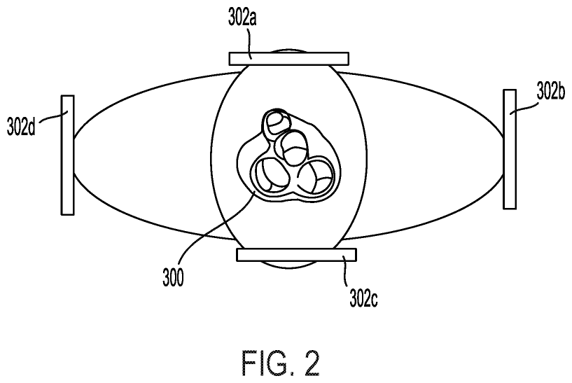

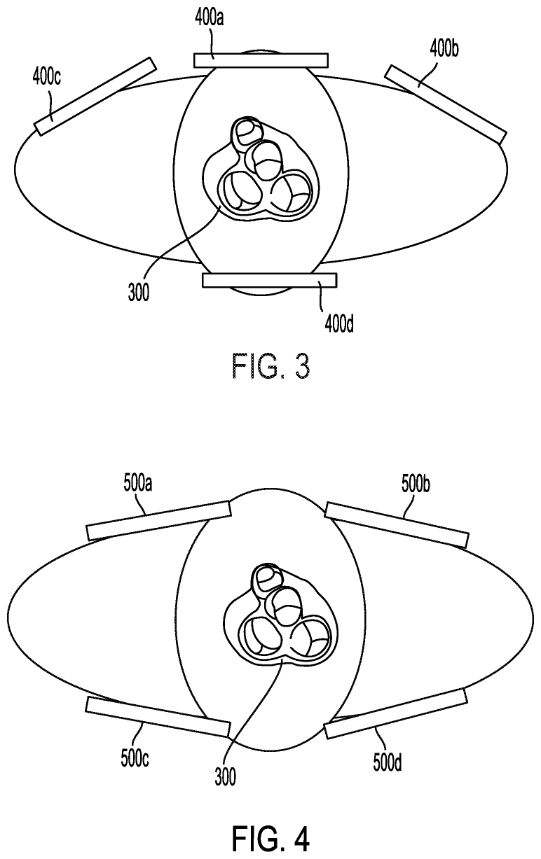

As an illustration, FIG. 2 shows an example of predetermined locations around a heart 300 of a patient for therapy electrodes 302a-302d. In the example of FIG. 2, a first therapy electrode 302a is configured to be disposed against a front torso location on a patient, a second therapy electrode 302b is configured to be disposed against a right side location on the patient, a third therapy electrode 302c is configured to be disposed against a back torso location on the patient, and a fourth therapy electrode 302d is configured to be disposed against a left side location on the patient (e.g., collectively, therapy electrodes 302). FIG. 3 illustrates another example of a therapy electrode placement that can be used to deliver vector sweeping defibrillation shocks. In the example of FIG. 3, three therapy electrodes 400a, 400b, and 400c are configured to be disposed on either a front or back torso location of a patient, and one therapy electrodes 400d is configured to be disposed on the other of a front or back torso location of the patient (collectively, therapy electrodes 400). FIG. 4 illustrates yet another example of a therapy electrode placement that can be used to deliver vector sweeping defibrillation shocks. In this example, the cardiac defibrillator includes therapy electrodes 500a and 500b that are configured to be disposed on first and second front torso locations of a patient and therapy electrodes 500c and 500d configured to be disposed on first and second back torso locations of the patient (collectively, therapy electrodes 500).

Other locations and/or numbers of therapy electrodes can be used to deliver vector sweeping defibrillation shocks. In examples, therapy electrodes may be disposed at predetermined locations on the patient that include at least one front torso location on the patient and at least one back torso location on the patient. In examples, various numbers of therapy electrodes may be used. For example, a cardiac defibrillator may include three therapy electrodes, four therapy electrodes, five therapy electrodes, six therapy electrodes, seven therapy electrodes, eight therapy electrodes, etc. For instance, a cardiac defibrillator may include six therapy electrodes configured to be disposed on three front torso locations and three back torso locations. As another illustration, a cardiac defibrillator may include eight therapy electrodes configured to be disposed on four front torso locations and four back torso locations. As another illustration, a cardiac defibrillator may include five or more therapy electrodes disposed substantially equidistantly from each other around the patient's torso. In implementations, the predetermined locations on the patient may include various anatomical locations across and around the patient's torso, for example, to accommodate a large number of therapy electrodes, such as more than ten.

In various embodiments, a cardiac defibrillator may be modified to allow for therapy electrode placements that better allow for the provision of vector sweeping defibrillation shocks to a patient. In examples, the wearable cardiac defibrillator 200 shown in FIG. 1A may be modified to create a wearable cardiac defibrillator better capable of providing vector sweeping defibrillation shocks. For instance, the wearable cardiac defibrillator 200 may be modified to allow for therapy electrode placement as shown in any of the configurations described above with respect to FIGS. 2-4. In examples, modifications may also be made to any of the other embodiments of cardiac defibrillators shown and described herein, such as the cardiac defibrillators discussed below with reference to FIGS. 19-21.

As an illustration, with respect to the wearable cardiac defibrillator 200 shown in FIG. 1A, the garment 208 of the wearable cardiac defibrillator 200 can be configured to support therapy electrodes in any of the configurations described above. For example, the garment 208 can be modified to support therapy components in any of the configurations shown in any of FIGS. 2-4. In such modified embodiments, the garment 208 may include additional and/or different enclosures or pockets to support additional and/or differently placed therapy electrodes from the embodiment shown in FIG. 1A. With reference to the configuration of FIG. 2, for instance, the garment 208 may be configured with an enclosure configured to be disposed at a front torso location of the patient (e.g., to house therapy electrode 302a), an enclosure configured to be disposed at a right side location of the patient (e.g., to house therapy electrode 302b), an enclosure configured to be disposed at a back torso location of the patient (e.g., to house therapy electrode 302c), and an enclosure configured to be disposed at a left side location of the patient (e.g., to house therapy electrode 302d). An example of this configuration is shown in FIG. 5D, discussed in further detail below. Alternatively, the garment 208 may be modified such that therapy electrodes 302 are permanently attached or adhered to the garment 208 in the locations shown in FIG. 2.

FIGS. 5A-5H illustrate example placements for therapy electrodes 600a-600nn (collectively, therapy electrodes 600) on a garment 602 of a wearable cardiac defibrillator. These example placements may be accommodated, for example, by enclosures configured to receive the therapy electrodes 600 and/or by permanent attachment of the therapy electrodes 600 to the garment 602. FIG. 5A shows example placements where therapy electrodes 600a and 600b are configured to be positioned on front locations of the patient's torso and where therapy electrodes 600c and 600d are configured to be positioned on a back location of the patient's torso. Similarly, FIG. 5B shows example placements where therapy electrodes 600e and 600f are configured to be positioned on front locations of the patient's torso and where therapy electrodes 600g and 600h are configured to be positioned on a back locations of the patient's torso (e.g., closer to the sagittal plane of the patient 202 compared to the position of the therapy electrodes 600c and 600d as shown in FIG. 5A). FIG. 5C shows example placements where therapy electrodes 600i, 600j, 600k, and 600m are all configured to be positioned on front portions of the patient's torso, with therapy electrodes 600k and 600m positioned superior to therapy electrodes 600i and 600j. FIG. 5D shows example placements where therapy electrode 600n is configured to be positioned on a front location on the patient's torso, therapy electrode 600p is configured to be positioned on a back location of the patient's torso, therapy electrode 600q is configured to be positioned on a right side location of the patient's torso, and therapy electrode 600u is configured to be positioned on a left side location of the patient's torso. For example, the therapy electrode 600q can be placed substantially about a middle axillary line on the right side of the patient. As another example, the therapy electrode 600u can be placed substantially about a middle axillary line on the left side of the patient. FIG. 5E shows example placements where therapy electrodes 600v and 600w are configured to be positioned on front locations of the patient's torso and therapy electrodes 600x and 600y are configured to be positioned on back locations of the patient's torso inferior to the therapy electrodes 600v and 600w.

FIG. 5F shows example placements where therapy electrodes 600z and 600aa are configured to be positioned on right side locations of the patient's torso (e.g., where therapy electrodes 600z and 600aa are placed side-by-side on the right side locations) and therapy electrodes 600bb and 600cc are configured to be positioned on left side locations of the patient's torso inferior to the therapy electrodes 600z and 600aa (e.g., where therapy electrodes 600bb and 600cc are placed side-by-side on the patient's left side locations). For example, the therapy electrodes 600z and 600aa can be placed on either side about a middle axillary line on the right side of the patient. As another example, the therapy electrodes 600bb and 600cc can be placed on either side about a middle axillary line on the left side of the patient. FIG. 5G shows example placements where therapy electrode 600dd is configured to be positioned on a right side location of the patient's torso, therapy electrode 600ee is configured to be positioned on a left side location of the patient's torso, and therapy electrodes 600ff and 600gg are configured to be positioned on back locations of the patient's torso. FIG. 5H shows example placements where therapy electrodes 600hh and 600ii are configured to be positioned on front locations of the patient's torso, therapy electrodes 600jj and 600kk are configured to be positioned against back locations of the patient's torso, therapy electrode 600mm is configured to be positioned again a right side location of the patient's torso, and therapy electrode 600nn is configured to be positioned against a left side location of the patient's torso. For example, the therapy electrode 600mm can be placed substantially about a middle axillary line on the right side of the patient. As another example, the therapy electrode 600nn can be placed substantially about a middle axillary line on the left side of the patient.

In implementations, the garment 602 is configured to receive the therapy electrodes 600 in the positions shown in FIGS. 5A-5H. As an illustration, the garment 602 may include enclosures configured to receive the therapy electrodes 600 as shown in FIGS. 5A-5H. Such enclosures may include, for instance, pockets. Thus, for the positions shown in FIGS. 5A, 5B, and 5E, the garment 602 may include two pockets configured to be positioned against front locations of the patient's torso. For example, the two pockets may be configured to receive therapy electrodes 600a and 600b in FIG. 5A. As another example, the two pockets may be configured to receive therapy electrodes 600e and 600f in FIG. 5B. As another example, the two pockets may be configured to receive therapy electrodes 600v and 600w in FIG. 5E. In implementations, the garment 602 may include two pockets configured to be positioned against back locations of the patient's torso. As an example, the two pockets may be configured to receive therapy electrodes 600c and 600d in FIG. 5A. As another example, the two pockets may be configured to receive therapy electrodes 600g and 600h in FIG. 5B. As another example, the two pockets may be configured to receive therapy electrodes 600x and 600y in FIG. 5E. For the positions shown in FIG. 5C, the garment 602 may include two pockets configured to be positioned against front locations of the patient's torso and configured to receive therapy electrodes 600i and 600j. The garment 602 may additionally include two pockets configured to be positioned against front locations of the patient's torso superior to the therapy electrodes 600i and 600j and configured to receive therapy electrodes 600k and 600m.

For the positions shown in FIG. 5D, the garment 602 may include a pocket configured to be positioned against a front location of the patient's torso and configured to receive therapy electrode 600n, a pocket configured to be positioned against a back location of the patient's torso and configured to receive therapy electrode 600p, a pocket configured to be positioned against a right side location of the patient's torso and configured to receive therapy electrode 600q, and a pocket configured to be positioned against a left side portion of the patient's torso and configured to receive therapy electrode 600u. For the positions shown in FIG. 5F, the garment 602 may include two pockets configured to be positioned against right side locations of the patient's torso and configured to receive therapy electrodes 600z and 600aa and two pockets configured to be positioned against left side locations of the patient's torso and configured to receive therapy electrodes 600bb and 600cc. For the positions shown in FIG. 5G, the garment 602 may include pockets configured to be positioned against back locations of the patient's torso and configured to receive therapy electrodes 600ff and 600gg, a pocket configured to be positioned against a right side location of the patient's torso and configured to receive therapy electrode 600dd, and a pocket configured to be positioned against a left side location of the patient's torso and configured to receive therapy electrode 600ee. For the positions shown in FIG. 5H, the garment 602 may include pockets configured to be positioned against front locations of the patient's torso and configured to receive therapy electrodes 600hh and 600ii, pockets configured to be positioned against back locations of the patient's torso and configured to receive therapy electrodes 600jj and 600kk, a pocket configured to be positioned against a right side location of the patient's torso and configured to receive therapy electrode 600mm, and a pocket configured to be positioned against a left side location of the patient's torso and configured to receive therapy electrode 600nn.

In implementations, instead of being configured with enclosures for the therapy electrodes 600, the garment 602 may be configured such that one or more of the therapy electrodes 600 shown in FIGS. 5A-5H is permanently attached at the respective location(s). For example, the garment 602 may be configured such that one or more of the therapy electrodes 600 is sewn into or permanently adhered at the respective location(s).

In implementations, the therapy electrode placements may be selected to account for male and female anatomy. For example, the electrode placements shown in FIGS. 5C and 5E including therapy electrodes 600 placed against upper front locations of the patient's torso may be used for male patients. The placement of the upper front therapy electrodes 600 may be adjusted for female patients to avoid interference from breast tissue. As an illustration, the upper front location therapy electrodes may be raised or moved to the patient's sides to avoid the therapy electrodes 600 being placed on top of the patient's breast tissue. In implementations, other positions of the therapy electrodes 600 may alternatively be used. For example, in implementations, the garment 602 may be configured to receive fewer than four or more than four therapy electrodes 600, such as the six therapy electrode 600 configuration shown in FIG. 5H.

In implementations, as discussed and shown above, a cardiac defibrillator may include three or more therapy electrodes. In implementations, a cardiac defibrillator may include four or more therapy electrodes. In implementations, the approximate predetermined locations on the patient for the therapy electrodes may be approximately 45 to 135 degrees radially from each other. In implementations, the approximate predetermined locations on the patient for the therapy electrodes may be approximately 60 to 120 degrees radially from each other. In implementations, the approximate predetermined locations on the patient for the therapy electrodes may be approximately 90 degrees radially from each other. As an example, a cardiac defibrillator may include eight electrodes positioned approximately 45 degrees radially from each other. As another example, a cardiac defibrillator may include six electrodes positioned approximately 60 degrees radially from each other. For instance, the predetermined locations for the six therapy electrodes may be similar to the locations for therapy electrodes 600hh-600nn shown in FIG. 5H. As another example, a cardiac defibrillator may include five electrodes positioned approximately 72 degrees from each other. As another example, a cardiac defibrillator may include four electrodes positioned approximately 90 degrees from each other. To illustrate, the predetermined locations for the four therapy electrodes may be similar to the locations for the therapy electrodes 600n-600u shown in FIG. 5D. As another example, a cardiac defibrillator may include three electrodes positioned approximately 120 degrees from each other. In implementations, the predetermined locations on the patient for the therapy electrodes may be a certain number of degrees radially from each other, plus or minus a certain amount of error. For example, the certain amount of error may be 10%, 15%, 20%, 25%, and/or the like.

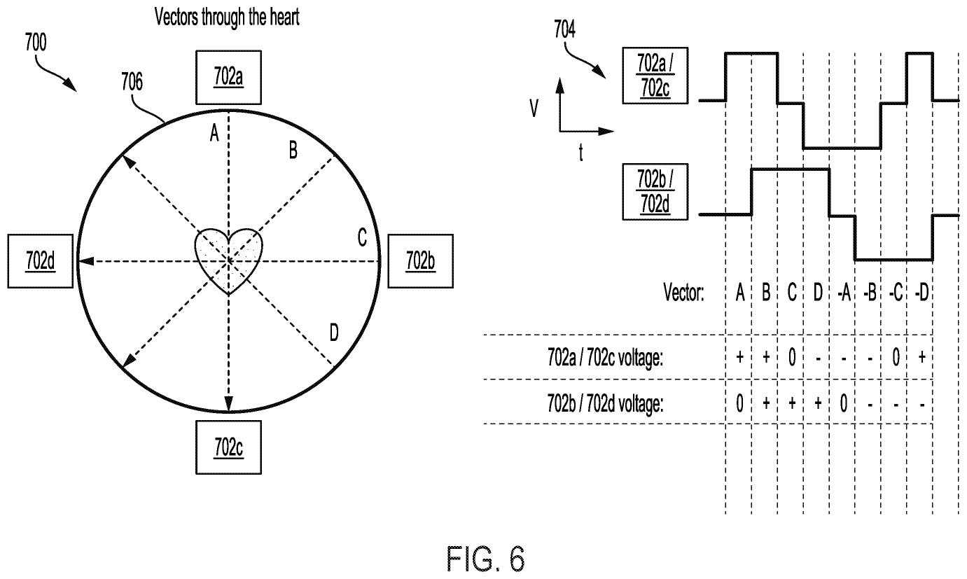

FIG. 6 illustrates an example of vector sweeping defibrillation shocks that can be delivered by a cardiac defibrillator using the therapy electrode placements shown in FIG. 2. Example vectors through a patient's heart 700 can be defined using two pairs of therapy electrodes: therapy electrodes 702a and 702c and therapy electrodes 702b and 702d (collectively, therapy electrodes 702). In the example of FIG. 6, the vectors through the patient's heart 700 include vector A defined by setting therapy electrode 702a to a high voltage and therapy electrode 702c to a low voltage, vector B defined by setting therapy electrodes 702a and 702b to a high voltage and therapy electrodes 702c and 702d to a low voltage, vector C defined by setting therapy electrode 702b to a high voltage and therapy electrode 702d to a low voltage, and vector D defined by setting therapy electrodes 702b and 702c to a high voltage and therapy electrodes 702d and 702a to a low voltage. Additional vectors-A, -B, -C, and -D can be defined by reversing the high/low voltages of vectors A, B, C, and D, respectively. As such, when the cardiac defibrillator including therapy electrodes 702 delivers vector sweeping defibrillation shocks, the wearable cardiac defibrillator may deliver a series of therapeutic shocks in a predetermined rotational sequence of vector A, vector B, vector C, vector D, vector A, vector-B, vector-C, and vector-D, as shown in vector sequence diagram 704. In this way, the cardiac defibrillator can deliver defibrillation shocks to the patient along a vector sweeping path 706 defined by the placement of the therapy electrodes 702 and the vectors A, B, C, D, -A, -B, -C, and -D that can be formed between them.

In implementations, the defibrillation vectors used in delivering the vector sweeping defibrillation shocks may include direct vectors and/or intermediate vectors. In implementations, direct vectors may be formed between therapy electrodes when the number of therapy electrodes set to a first polarity (e.g., a high voltage or a low voltage) is the same as the number of therapy electrodes set to a second polarity (e.g., the other of a high or low voltage). For example, a direct vector may be a two-electrode direct vector between a set of two therapy electrodes. A cardiac defibrillator may deliver a defibrillation shock along each two-electrode direct vector by setting a first member of the set of two therapy electrodes to a first polarity (e.g., a high voltage or a low voltage) and setting the other member of the set of two therapy electrodes to a second polarity (e.g., the other of the high voltage or low voltage). To illustrate, vectors A and -A are two-electrode direct vectors in FIG. 6, formed between therapy electrodes 702a and 702c. Similarly, vectors C and -C are two-electrode direct vectors in FIG. 6, formed between therapy electrodes 602b and 602d. Such two-electrode direct vectors may be referred to as first order vectors.

As another example, a direct vector may be a four-electrode direct vector formed from a four-electrode set of two pairs of therapy electrodes. The cardiac defibrillator is configured to deliver a therapeutic shock along each four-electrode direct vector by setting a first electrode pair from the four-electrode set to a first polarity (e.g., a high voltage or a low voltage) and setting the other electrode pair from the four-electrode set to a second polarity (e.g., the other of the high or low voltage). For instance, vectors B, D, -B, and -D are four-electrode direct vectors in FIG. 6, created by therapy electrodes 702. Within the therapy electrodes 702, a first pair of the therapy electrodes 702 are configured to be disposed at adjacent predetermined locations on the patient and are set to a first polarity. A second pair of the therapy electrodes 702 are also configured to be disposed at adjacent predetermined locations on the patient and are set to a second polarity. For example, to create vector B, therapy electrodes 702a and 702b serve as the first pair and are set to a high voltage, and therapy electrodes 702c and 702d serve as the second pair and are set to a low voltage. Conversely, to create vector D, therapy electrodes 702b and 702c serve as the first pair set to a high voltage, and therapy electrodes 702a and 702d serve as the second pair set to a low voltage. Such four-electrode direct vectors may be referred to as second-order vectors. In implementations, second-order direct vectors may be formed between therapy electrodes when the numbers of electrodes set to a first polarity and a second polarity are the same and the voltages used are also the same between the sets of electrodes. For instance, a direct vector B may be formed when electrodes 702a and 702b are set to the same positive voltage polarity and electrodes 702c and 702d are set to the same negative voltage polarity.

In implementations, intermediate vectors may be formed when the number of therapy electrodes set to a first polarity (e.g., a high voltage or a low voltage) is not the same as the number of therapy electrodes set to a second polarity (e.g., the other of a high or low voltage). For example, an intermediate therapy vector may be formed from at least three therapy electrodes. Other intermediate therapy vectors may be formed from other, odd numbers of therapy electrodes. Within the therapy electrodes configured to form an intermediate therapy vector, a first set within the therapy electrodes (e.g., including at least two electrodes) are configured to be disposed at adjacent predetermined locations on the patient. The remaining set of therapy electrodes may be a solo therapy electrode or multiple therapy electrodes also configured to be disposed at adjacent predetermined locations on the patient separate from the locations of the first set, where the first set and the second set have different numbers of therapy electrodes. For instance, in an intermediate vector formed from three therapy electrodes, two therapy electrodes may be disposed at adjacent locations on the patient, and the third therapy electrode may be disposed across the patient's torso from the other two therapy electrodes. The cardiac defibrillator may deliver a defibrillation shock along an intermediate vector by setting the members of the first set within the therapy electrodes to a first polarity (e.g., a high voltage or a low voltage) and may set the remaining member or members of therapy electrodes to a second polarity from the first set (e.g., the other of the high or low voltage).

As an illustration, referring to FIG. 6, an intermediate AB vector may be formed using therapy electrodes 702a, 702b, and 702c. The cardiac defibrillator may set therapy electrodes 702a and 702b to a high voltage and therapy electrode 702c to a low voltage. The direction of the intermediate AB vector may be determined by summing the individual vectors between the therapy electrodes 702a and 702c and between the therapy electrodes 702b and 702c. Other intermediate vectors can be formed with the therapy electrode configuration shown in FIG. 6. For instance, an intermediate BC vector can be formed by setting therapy electrodes 702a and 702b to a high voltage and therapy electrode 702d to a low voltage. An intermediate CD vector can be formed by setting therapy electrodes 702b and 702c to a high voltage and therapy electrode 702d to a low voltage. An intermediate D-A vector can be formed by setting therapy electrodes 702b and 702c to a high voltage and therapy electrode 702a to a low voltage, and so on. As an example, a cardiac defibrillator may deliver vector sweeping therapeutic shocks to a patient along vector A, vector AB, vector B, vector BC, vector C, vector CD, vector D, vector D A, vector-A, vector-A-B, vector-B, vector-B-C, vector-C, vector-C-D, vector-D, and vector DA.

In implementations, intermediate vectors may be formed between therapy electrodes when the numbers of electrodes set to a first polarity and a second polarity are the same but the voltages used are also the same between the sets of electrodes. As an example, an intermediate vector may be formed by setting electrode 702a to a first positive voltage polarity, electrode 702b to a second positive voltage polarity, electrode 702d to a first negative voltage polarity corresponding to the first positive voltage polarity, and electrode 702c to a second negative voltage polarity corresponding to the second positive voltage polarity.

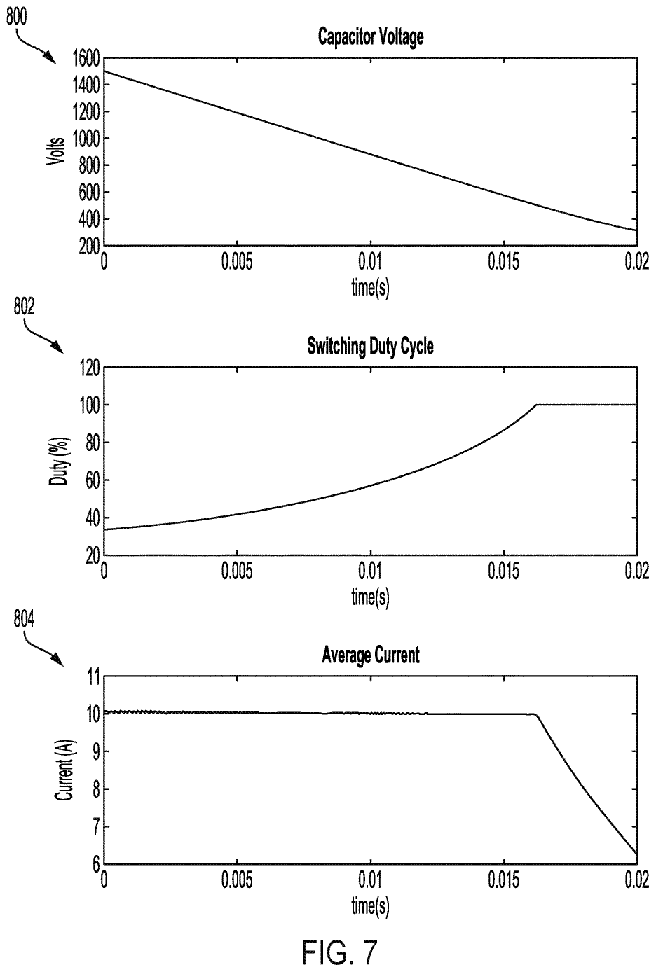

In implementations, more granular resolution for vector sweeping defibrillation shocks may be provided using pulse width modulation to shape the defibrillation waveforms. FIG. 7 illustrates an example of using pulse width modulation to maintain a constant current (e.g., a constant average current) from a capacitor. In aspects, the constant current may be delivered to the patient based on the fact that, even though the defibrillation waveforms may not be perfectly smooth, the cardiomyocytes integrate current pulses if they are faster than 5 Hz. For example, if sampled on an order of around 100 microseconds or more, the number of electrons flowing through the cardiomyocytes according to the techniques described herein may be roughly constant, and the “sampling period” of cardiomyocytes may be on the order of around 200 microseconds.

Pulse width modulation may be controlled by a defibrillation controller, such as the defibrillation controller 214, the defibrillation controller 250, and/or any of the defibrillation controller implementations described below with respect to FIGS. 19-21. Rather than turning the capacitor on at full capacity, the defibrillation controller modulates the duty cycle of the capacitor or bank of capacitors (e.g., the percentage of time the capacitor is on over time). Accordingly, FIG. 7 illustrates a duty cycle graph 802 for the capacitor, plotting duty cycle (%) over time(s). As shown in FIG. 7, the duty cycle for the capacitor starts around 35% and grows exponentially over time. A corresponding capacitor voltage graph 800 of capacitor voltage (V) over time(s) illustrates that the capacitor voltage starts at around 1500 V. However rather than the capacitor discharging at full capacity, due to the exponential nature of the duty cycle over time, the capacitor voltage decreases linearly over time. An average current graph 804 of current (A) over time(s) shows that, because the capacitor voltage decreases linearly, the average current remains roughly constant over time until the capacitor is nearly entirely discharged after 0.015 seconds. In implementations, the defibrillation controller modulates the duty cycle by rapidly varying the pulse width of how the capacitor is discharged. In implementations, the defibrillation controller may alternatively or additionally use similar methods to modulate the frequency of pulses from the capacitor as part of applying pulse frequency modulation.

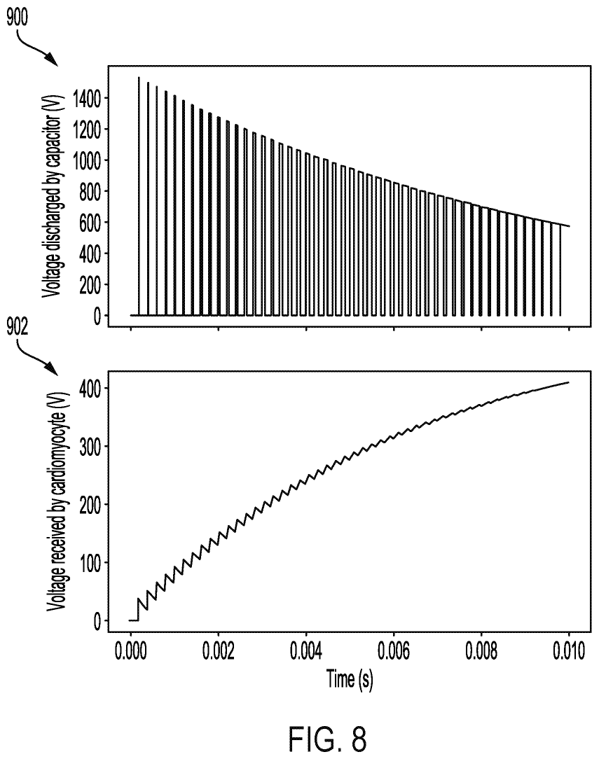

However, a defibrillation controller may modulate duty cycle over time according to a variety of waveforms to create a variety of corresponding voltage and average current waveforms. FIG. 8 shows another example of a defibrillation controller using pulse width modulation to shape the discharge of a capacitor. More specifically, FIG. 8 illustrates the discharge of a capacitor and the corresponding voltage that is received by cardiomyocytes upon delivery to the patient. For example, the defibrillation controller may be defibrillation controller 214, the defibrillation controller 250, and/or any of the defibrillation controller implementations described below with respect to FIGS. 19-21. FIG. 8 includes a capacitor voltage graph 900 of voltage (V) discharged from the capacitor over time(s) and a received voltage graph 902 of voltage (V) received by the cardiomyocytes during defibrillation treatment. As illustrated in FIG. 8, the capacitor discharges according to a duty cycle that increases over time as the remaining capacitor voltage decreases. In the example of FIG. 8, the defibrillation controller modulates the duty cycle by increasing the pulse width of pulses from the capacitor as the capacitor discharges and the available voltage drops. Accordingly, the voltage received by the cardiomyocytes over time follows a partial sine wave. In other examples, the defibrillation controller may additionally or alternatively modulate the frequency of pulses from the capacitor. For instance, the defibrillation controller may increase the frequency of pulses from the capacitor as the capacitor discharges to produce a similar voltage received waveform.

This principle can be extended to applying defibrillation shocks to a patient via a cardiac defibrillator, such as the wearable cardiac defibrillator 200 and/or the cardiac defibrillators discussed below with respect to FIGS. 19-21. As an illustration, FIG. 9 shows example graphs of using pulse modulation waveforms to shape vector sweeping defibrillation shocks delivered to a patient. More particularly, FIG. 9 includes a therapy electrode voltage graph 1000 of voltage (V) over time (ms) and a duty cycle graph 1002 of duty cycle (%) over time (ms) for a cardiac defibrillator with four therapy electrodes. The therapy electrode voltage graph 1000 includes voltage waveforms for a first therapy electrode 1004, a second therapy electrode 1006, a third therapy electrode 1008, and a fourth therapy electrode 1010. As shown in FIG. 9, each of the voltage waveforms 1004-1010 is configured as a half sine wave (with the exception of the voltage waveform for the first therapy electrode 1004, where the half sine wave is split into two portions). Additionally, as further illustrated in FIG. 9, the timings of the voltage waveforms 1004-1010 overlap with each other. For instance, in the example of FIG. 9, each of the voltage waveforms 1004-1010 begins halfway through the previous waveform 1004-1010. Accordingly, the waveform for the second therapy electrode 1006 begins at the apex of the waveform for the first therapy electrode 1004 (e.g., given that the waveform for the first therapy electrode 1004 is split into two portions), the waveform for the third therapy electrode 1008 begins at the apex of the waveform for the second therapy electrode 1006, and so on.

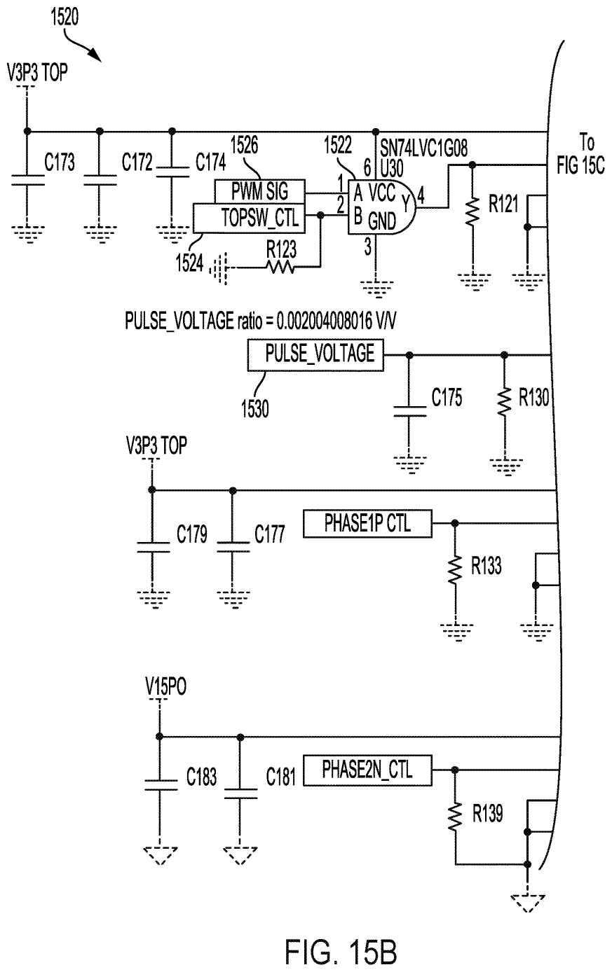

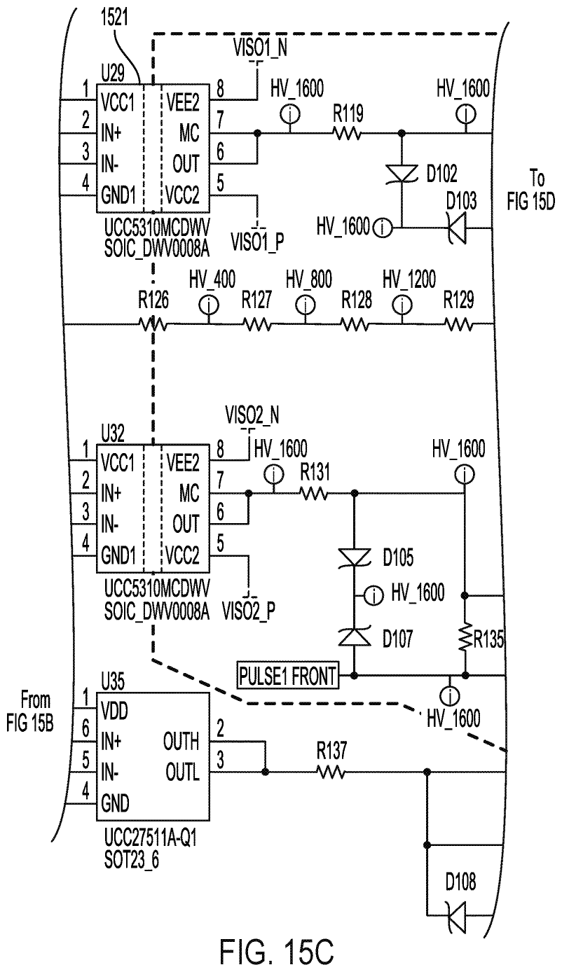

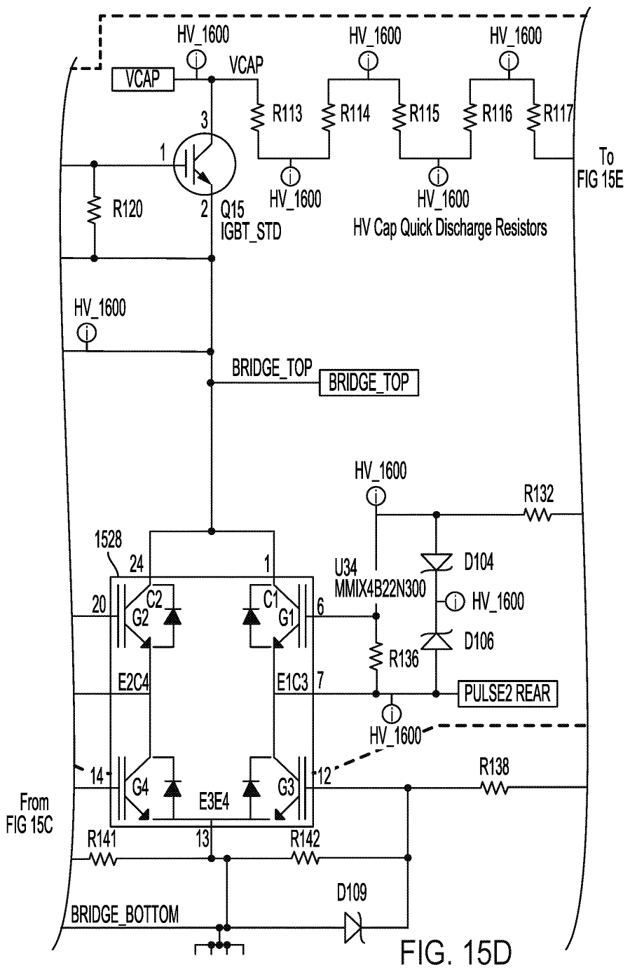

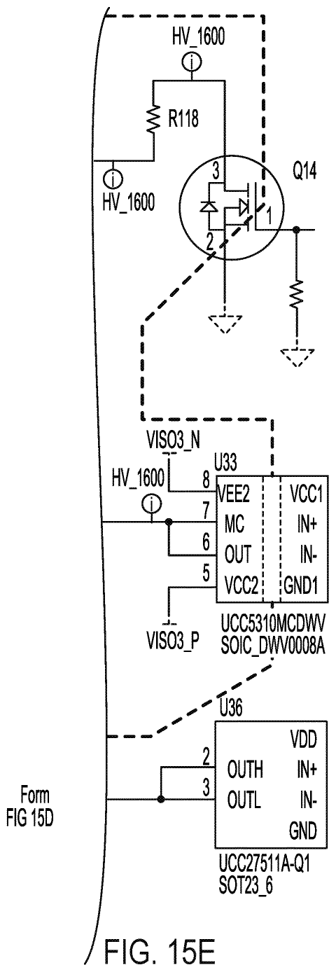

The shape of each of the voltage waveforms 1004-1010 is provided by corresponding duty cycle waveforms, as shown in the duty cycle graph 1002. For example, in implementations, the voltage waveforms 1004-1010 are generated by applying pulse width modulation to an insulated-gate bipolar transistor (IGBT) gate control electrically connected to the therapy electrodes. The pulse width modulation results in the IGBT turning on and off for a brief but defined period of time in a sequence. By varying the length of time “on” versus time “off” (e.g., by varying the duty cycle according to a duty cycle plot or waveform), the net output voltage can be controlled and shaped as described above. As an illustration, the duty cycle graph 1002 includes duty cycle waveforms 1012 and 1014, where the duty cycle waveform 1012 corresponds to the first and third therapy electrodes and the duty cycle waveform 1014 corresponds to the second and fourth therapy electrodes. In examples, the duty cycle waveforms and 1012 and 1014 are reduced down to two waveforms for the four electrodes, as shown in FIG. 9, due to switches for the therapy electrodes that are not on at the same time being combined into a single waveform. Examples of these switches are shown and described below with reference to FIGS. 15A and FIGS. 15B-15E. Each of the duty cycle waveforms 1012 and 1014 is configured as a series of half sine waves that are slightly slanted, with the waveform 1012 for the first and third therapy electrodes also including a first half sine wave that is truncated.

As a result of the therapy electrode capacitors being discharged according to the duty cycle waveforms 1012 and 1014, the defibrillation controller can create the voltage waveforms 1004-1010. Accordingly, the defibrillation controller of a cardiac defibrillator modulating vector sweeping waveforms of the vector sweeping defibrillation shock can allow for modulating a therapy electrode voltage for each of the therapy electrodes of the cardiac defibrillator. For example, with reference to FIG. 9, modulating the therapy electrode voltage for each of the therapy electrodes may include modulating the therapy electrode voltage according to an approximate sine wave.

In implementations, a peak of each approximate sine wave of a defibrillation voltage waveform may be between 400 V and 1250 V. In implementations, a peak of each approximate sine wave of a defibrillation voltage waveform may be between 400 V and 800 V. In implementations, a patient caregiver such as a healthcare provider or a service representative may be able to input the peak voltage for the defibrillation controller to use. For example, a patient caregiver may be able to input a peak voltage for the defibrillation controller using an input device on or operably connected to the defibrillation controller. To illustrate, a patient caregiver may be able to input a peak voltage using the user interface 260 of the defibrillation controller 250. In examples, a patient caregiver may instead input a target current for the defibrillation controller, with the defibrillation controller automatically determining other settings for the defibrillation waveforms using the target current. The other settings may include, for example, the peak voltage of voltage waveforms, the shape of the duty cycle waveforms, and/or the like.

In implementations, to generate the duty cycle waveforms, the time-varying voltage of the capacitors of the therapy electrodes and the load impedance must be known. The pulse width modulation compensates for decaying capacitor voltage to ensure the output voltage is the desired value to result in the correct load current. As an example, below is a generic version of the pulse width modulation waveform, where PWM(t) is the pulse width modulation (PWM) duty cycle output for a pair of therapy electrodes as a percentage over time, I_{load} is the desired load current (A), R is the desired load resistance (Ω), VC(t) is the capacitor voltage over time, and Vₜ is the vector time. In implementations, the total vector time is 8×Vₜ.

$$P W M ( t ) = [ \frac{{I}_{l o a d} * R}{{V}_{C} ( t )} ] * \vert sin ( \frac{π * t}{4 * {V}_{t}} ) \vert$$

The electronic architecture used to control the vector sweeping defibrillation shocks is also described in further detail below with respect to FIGS. 15A and FIGS. 15B-15E. In implementations, the above represents a generic formula for each individual electrode when attempting to deliver a constant radial current PWM. This formula describes the instantaneous duty cycle required to maintain a half wave sinusoidal output voltage. However, when comparing the individual therapy electrodes to each other, this formula would also include a time and phase delay to encompass the timing of when each individual therapy electrode delivers a shock to the patient, as the timings of the therapy electrodes are offset from each other (e.g., as shown in FIG. 9).

In implementations, the therapy electrodes used to produce the voltage waveforms 1004-1010 may be provided at predetermined locations approximately equidistant from each other around the patient's body. As an illustration, the therapy electrodes may be spaced radially even to each other, and therefore the vector sweeping defibrillation waveforms delivered to the patient may be spaced radially even to each other (e.g., as shown in FIG. 9). In examples, the therapy electrodes may be spaced radially even to each other at approximately 60 to 120 degree intervals. Accordingly, the defibrillation waveforms delivered by the therapy electrodes may be spaced radially even to each other at approximately 60 to 120 degree intervals. Such defibrillation waveforms may include, for instance and as discussed above, voltage waveforms shaped as an approximate sine wave or half sine wave. To illustrate, the therapy electrodes may be spaced radially even to each other at approximately 90 degrees to each other around the patient's body. For example, the therapy electrodes may be provided at the predetermined locations shown in FIG. 2 with therapy electrodes 302. As another example, the therapy electrodes may be provided at the predetermined locations shown in FIG. 5D with therapy electrodes 600n-600u. In such examples, the defibrillation waveforms delivered by the therapy electrodes may be spaced approximately 90 degrees to each other radially around the patient's body.

In other illustrations, the defibrillation waveforms shown in FIG. 9 may be adapted to a different number of therapy electrodes, such as by including additional or fewer defibrillation waveforms for additional or fewer numbers of therapy electrodes. For example, a defibrillation controller may deliver defibrillation waveforms shaped similarly to those shown in FIG. 9 but where the defibrillation waveforms include six waveforms for six therapy electrodes. In such examples, the six therapy electrodes may be placed radially even to each other at approximately 60-degree intervals. As such, the defibrillation waveforms may be delivered to the patient at approximately 60-degree intervals.

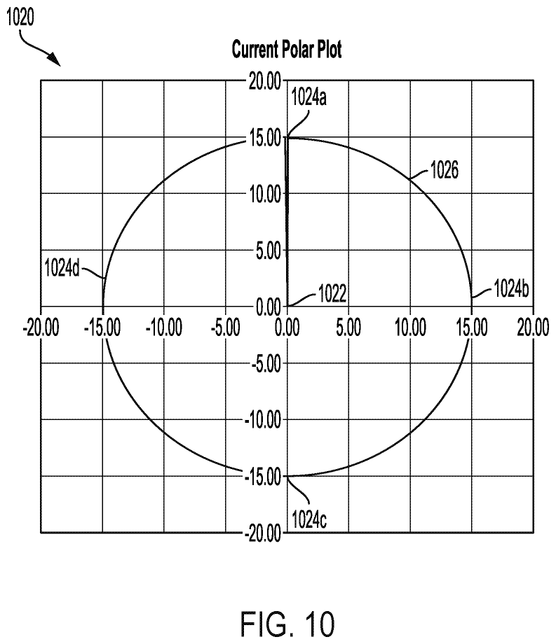

FIG. 10 shows an example of a resulting polar current plot 1020 from the voltage waveforms 1004-1010 of FIG. 9, with the therapy electrodes placed at predetermined, equidistant locations (e.g., approximately 90 degrees from each other around the patient's body). In the example of FIG. 10, a center 1022 of the polar current plot 1020 represents the center of the patient's body as seen looking down on a transverse plane bisecting the patient's body. The therapy electrodes may be positioned at predetermined locations 1024a, 1024b, 1024c, and 1024d (collectively, predetermined locations 1024). The therapy electrodes at the predetermined locations 1024 define the vector sweeping path for current around the patient's torso, as represented by a graph of the current 1026.

As illustrated in FIG. 10, the result of the voltage waveforms 1004-1010 is an approximately constant current 1016 around the patient's body along the vector sweeping path. The vector sweeping path may be, as an illustration, a 360-degree path in a transverse or angled plane about a vertical axis through the patient's torso. For instance, as shown, the approximately constant current 1026 creates an approximate circle around the patient's body. In examples, an approximately constant current 1016 may include current readings being within 5% of each other around the majority of the vector sweeping path. In examples, an approximately constant current 1026 may include current readings being within 10% of each other around the majority of the vector sweeping path. In examples, an approximately constant current 1026 may include current readings being within 15% of each other around the majority of the vector sweeping path. In examples, an approximately constant current 1026 may include current readings being within 20% of each other around the majority of the vector sweeping path. In examples, the approximately constant current may have reduced variability of voltage or current delivered via the vector sweep waveform by an order of magnitude compared to non-PWM methods.

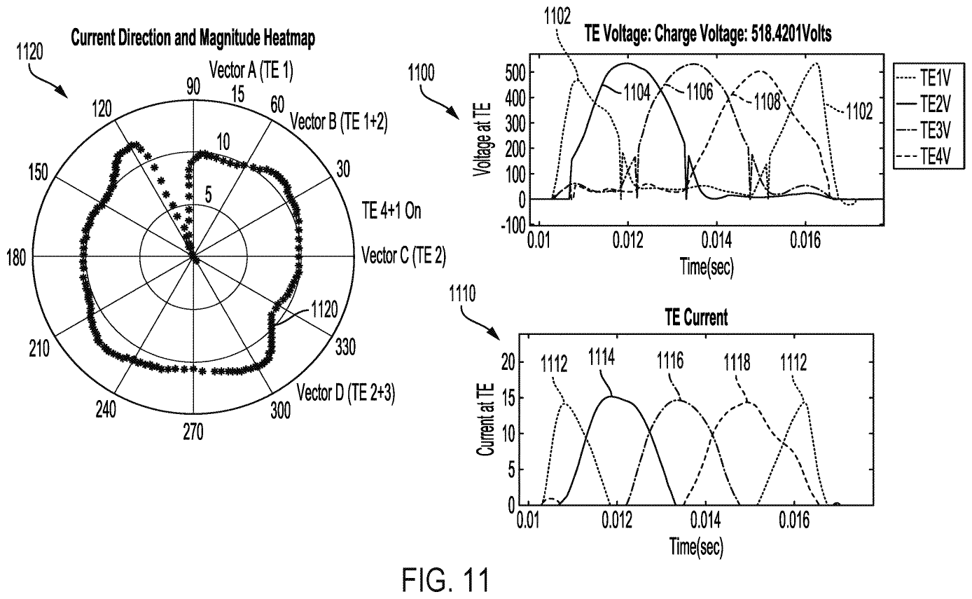

In various implementations, the approximately constant current 1016 shown in FIG. 10 may be an idealizes representation of the current delivered by the therapy electrodes according to the graphs shown in FIG. 9. During actual therapy delivery, the constant current 1016 may be affected by external factors, such as patient impedance changes (e.g., due to the patient moving during energy delivery), system response time, the overall energy required, and/or the like. As a further illustration, FIG. 11 shows a model of the voltage waveforms 1004-1010 being applied to a patient. The example of FIG. 11 includes a therapy electrode voltage graph 1100 of the voltage (V) applied by four modeled therapy electrodes over time(s). In the voltage graph 1100, a voltage waveform for a first therapy electrode 1102 corresponds to the voltage waveform for the first therapy electrode 1004 of FIG. 9, a voltage waveform for a second therapy electrode 1104 corresponds to the voltage waveform for the second therapy electrode 1006, a voltage waveform for a third therapy electrode 1106 corresponds to the voltage waveform for the third therapy electrode 1008, and a voltage waveform for a fourth therapy electrode 1108 corresponds to the voltage waveform for the fourth therapy electrode 1010. FIG. 11 also includes a therapy electrode current graph 1110 of the current (A) applied by the four modeled therapy electrodes over time(s). As shown, the therapy electrode current graph 1110 includes current waveforms for each of the four electrodes, including a current waveform for the first therapy electrode 1112, a current waveform for the second therapy electrode 1114, a current waveform for the third therapy electrode 1116, and a current waveform for the fourth therapy electrode 1118. Each of the current waveforms 1112-1118 is in a similar shape to the corresponding voltage waveform 1102-1108.

FIG. 11 further illustrates a polar current plot 1120 for the modeled vector sweeping defibrillation shocks applied to the patient. Similar to the polar current plot 1020 shown in FIG. 10, as modeled, the vector sweeping defibrillation shocks produce approximately constant current along the vector sweeping path around the patient's torso. The approximately constant current is demonstrated by the approximate circle the plotted current creates in the polar current plot 1020. For example, the target constant current along the vector sweeping path around the patient's torso for FIG. 11 was approximately 10 A. However, in implementations, the target current for the vector sweeping defibrillation shocks may be, for example, between 3 A and 75 A. In examples, a defibrillation controller may be set to provide an average current between 3 A and 75 A around the vector sweeping defibrillation path using pulse wave modulation. In examples, a defibrillation controller may be set to provide an average current between 5 A and 20 A around the vector sweeping defibrillation path using pulse wave modulation.

In implementations, a patient caregiver such as a healthcare provider or a service representative may be able to input a target current for the defibrillation controller. For example, a patient caregiver may be able to input a target current for the defibrillation controller using an input device on or operably connected to the defibrillation controller. To illustrate, a patient caregiver may be able to input a target current using the user interface 260 of the defibrillation controller 250. Such target current may be based, for example, on impedance measurements taken for the patient, on the patient's body mass index (BMI), and/or the like.

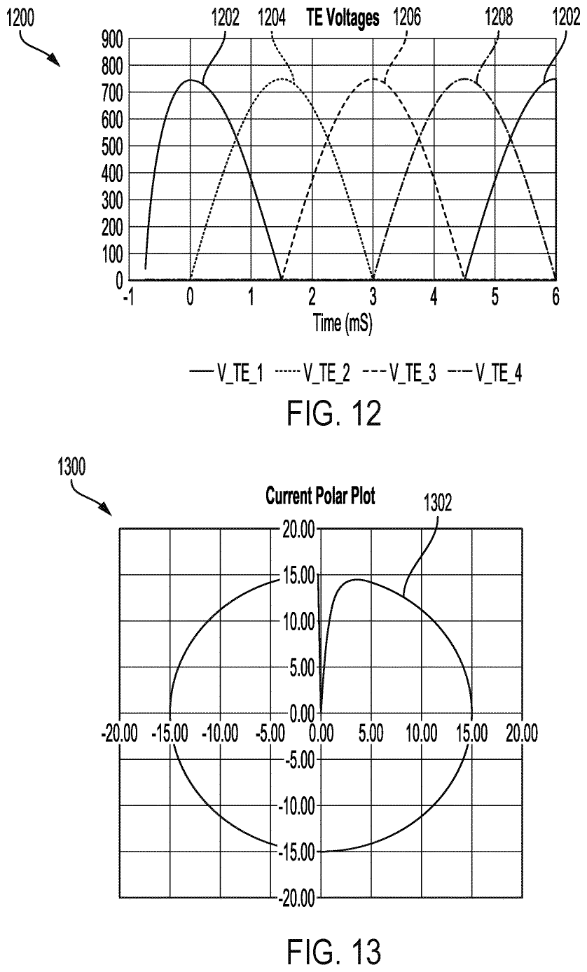

A defibrillation controller may use pulse width modulation to produce other variations of, for example, the voltage waveform shown in FIGS. 9 and 11. For instance, FIG. 12 illustrates another example of a therapy electrode voltage graph 1200. Similar to the therapy electrode voltage graph 1000, the therapy electrode voltage graph 1200 of FIG. 12 includes voltage waveforms for a first therapy electrode 1202, a second therapy electrode 1204, a third therapy electrode 1206, and a fourth therapy electrode 1208. Additionally, similar to the voltage waveforms 1004-1010, the voltage waveforms 1204-1208 are shaped as half sine waves with timings that are offset from each other (e.g., including the voltage waveform for the first therapy electrode 1202 being split into two portions). For example, as shown in FIG. 12, each of the voltage waveforms 1204-1208 begins approximately at the halfway point of the previous voltage waveform 1204-1208. However, unlike the voltage waveform for the first therapy electrode 1004 shown in FIG. 9, the voltage waveform for the first therapy electrode 1202 includes a leading edge that forms a ramp at the start of the voltage waveform 1202. Including the ramp at the first voltage waveform 1202 eliminates the sharp initial edge of the defibrillation shocks applied to the patient, which may reduce the incidence of tissue damage.

For example, in implementations and as shown in FIG. 12, the ramp starts at t=−ramp_time. The first voltage waveform 1202 thus achieves peak voltage at t=0, and the vector sweep begins as the second therapy electrode starts to energize according to the second voltage waveform 1204. With respect to the example formula for pulse width modulation discussed above, the ramp is achieved by adding a ramp term to the formula, as shown below.

$$PWM ( t ) = ( 1 - {e}^{( \frac{5 * ( t + {r}_{t} )}{{r}_{t}} )} ) [ \frac{{I}_{l o a d} * R}{{V}_{C} ( t )} ] * \vert sin ( \frac{π * t}{4 * {V}_{t}} ) \vert$$

Compared to the previous formula, this formula includes an exponential term that produces the ascending ramp, where rt represents the ramp time and the t+rₜ term produces an offset so that the ramp occurs before the rest of the pulse width modulation.

Alternatively, in implementations, rather than occurring before the defibrillation begins to sweep around the patient's torso along the vector sweeping path, the ramp may occur during the vector sweep. As an example, with reference to the previous formula, the t+rₜ term would simply be t as shown below.

$$P W M ( t ) = ( 1 - {e}^{( \frac{5 * t}{{r}_{t}} )} ) [ \frac{{I}_{l o a d} * R}{{V}_{C} ( t )} ] * \vert sin ( \frac{π * t}{4 * {V}_{t}} ) \vert$$

FIG. 13 illustrates a polar current plot 1300 for the situation in which the ramp occurs during the vector sweep. As shown, the ramp manifests as a notch 1302 in the current at the vertical axis (e.g., near where the first therapy electrode would be placed). In implementations, a patient caregiver may be able to input to a defibrillation controller whether the vector sweeping path includes an initial rising waveform and/or where the initial rising waveform occurs. For example, a patient caregiver may be able to input to the defibrillation controller that the vector sweeping defibrillation shocks should include an initial rising waveform that ramps up before the sweeping nature of the defibrillation shocks initiate.

Accordingly, by using pulse width modulation, a defibrillation controller may deliver a vector sweeping defibrillation shock, via a number of therapy electrodes, along vectors across the patient's torso where the vectors are configured with fine angular resolution and smooth transitions between adjacent vectors. Such defibrillation shocks may provide for better patient outcomes by maximizing the vector angles by which defibrillation energy is delivered to the patient, which in turn maximizes the chances of depolarizing cardiomyocytes in various orientations. For example, as shown above with respect to FIGS. 10, 11, and 13, the vector sweeping path may be in a 360-degree path around a vertical axis of the patient's torso, thereby delivering current in 360 degrees around the patient's heart. Additionally, the smooth transitions between adjacent vectors may allow for more seamless and patient-friendly defibrillation delivery (e.g., less likely to burn the patient's skin during delivery).

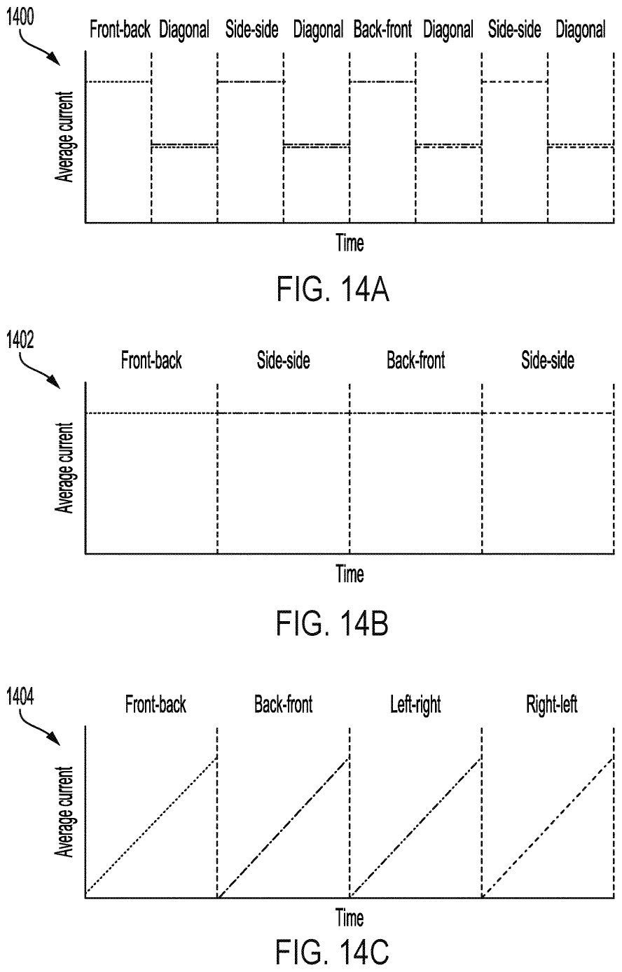

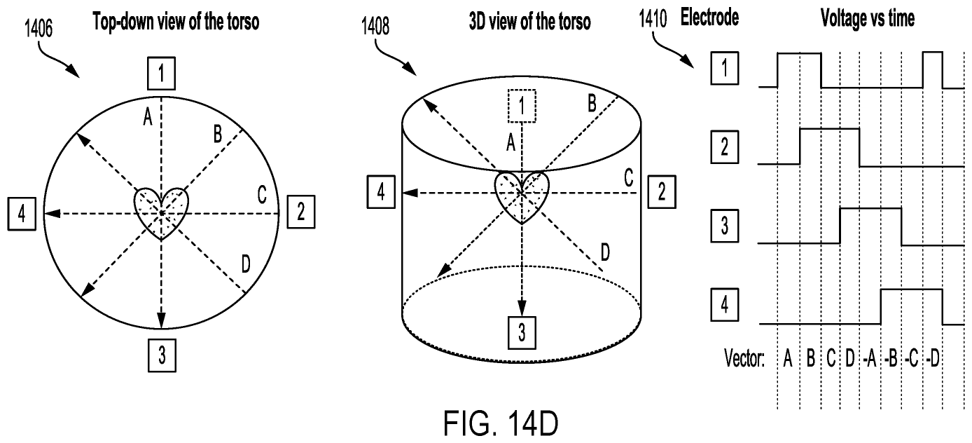

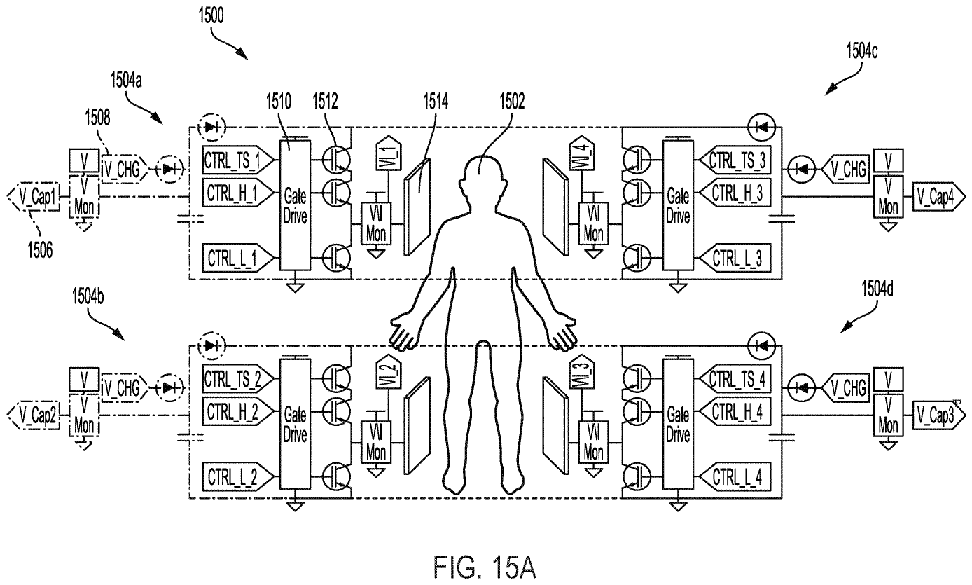

As shown above, in implementations, a defibrillation controller may deliver voltage waveforms to a patient during defibrillation therapy where the voltage waveforms are configured as half sinusoidal or approximately half sinusoidal waveforms. In implementations, a defibrillation controller may use pulse width modulation to create other types and shapes of voltage and current waveforms as part of delivering vector sweeping defibrillation shocks to the patient. FIGS. 14A-14C illustrate additional examples of therapy electrode current graphs that can be generated by a defibrillation controller. Examples of the defibrillation controller include the defibrillation controller 214, the defibrillation controller 250, and/or the defibrillation controllers discussed below with respect to FIGS. 19-21. For example, a defibrillation controller may generate the therapy electrode current graphs shown in FIGS. 14A-14C for vector sweeping defibrillation shocks delivered by four electrodes disposed at predetermined locations around a patient's torso. Such predetermined locations may be, for instance, approximately equidistant around the patient's torso (e.g., at approximately 90 degrees from each other around the patient's torso). As an illustration, a first therapy electrode may be positioned at a predetermined location on the front of the patient's torso, a second therapy electrode may be positioned at a predetermined location on the back of the patient's torso, and third and fourth therapy electrodes may be positioned on each of the patient's left and right sides. In examples, the four electrodes may be at predetermined locations around the patient's torso as shown with respect to the therapy electrodes 302 shown in FIG. 2. In examples, the four electrodes may be at predetermined locations around the patient's torso as shown with respect to the therapy electrodes 600n-600u shown in FIG. 5D. In implementations, adaptations may be made to the therapy electrode current graphs shown in FIGS. 14A-14C for different numbers of electrodes positioned approximately equidistant around the patient's torso, such as three electrodes, five electrodes, six electrodes, and/or the like.

FIG. 14A illustrates an example therapy electrode current graph 1400 where the defibrillation controller uses pulse width modulation to generate constant current for each of a series of therapy electrode vectors. In implementations, the therapy electrode vectors may resemble the vectors shown in FIG. 6 and described above. For example, the defibrillation controller generates, in sequence, (1) a first, constant level of current using the front and back therapy electrodes (e.g., analogous to vector A in FIG. 6), (2) a second, constant, lower level of current along a diagonal using all four electrodes (e.g., analogous to vector B in FIG. 6), (3) the first level of current using the left side and right side therapy electrodes (e.g., analogous to vector C in FIG. 6), (4) the second level of current along another diagonal using all four electrodes (e.g., analogous to vector D in FIG. 6), (5) the first level of current using the back and front therapy electrodes (e.g., analogous to vector-A in FIG. 6), (6) the second level of current along another diagonal using all four electrodes (e.g., analogous to vector-B in FIG. 6), (7) the first level of current using the right side and left side therapy electrodes (e.g., analogous to vector-C in FIGS. 6), and (8) the second level of current along another diagonal using all four electrodes (e.g., analogous to vector-D in FIG. 6). In implementations, the sequence may be generated in the opposite direction (e.g., counterclockwise instead of clockwise around the patient's torso).

FIG. 14B illustrates another example therapy electrode current graph 1402 where the defibrillation controller uses pulse width modulation to generate constant current for each of a series of therapy electrode vectors. In the example of FIG. 14B, the defibrillation controller generates, in sequence, (1) a constant level of current using the front and back therapy electrodes (e.g., analogous to vector A in FIG. 6), (2) the same constant level of current using the left side and right side therapy electrodes (e.g., analogous to vector C in FIG. 6), (3) the same constant level of current using the back and front therapy electrodes (e.g., analogous to vector-A in FIGS. 6), and (4) the same constant level of current using the right side and left side therapy electrodes (e.g., analogous to vector-C in FIG. 6). In implementations, the sequence may be generated in the opposite direction (e.g., counterclockwise instead of clockwise around the patient's torso).

FIG. 14C illustrates another example therapy electrode current graph 1404. In the therapy electrode current graph 1404, the defibrillation controller uses pulse width modulation to generate a linearly increasing amount of current for each of a series of therapy electrode vectors. For instance, as shown, the defibrillation controller generates in a sequence (1) a linearly increasing current using the front and back therapy electrodes (e.g., analogous to vector A in FIG. 6), (2) a linearly increasing current using the left side and right side therapy electrodes (e.g., analogous to vector C in FIG. 6), (3) a linearly increasing current using the back and front therapy electrodes (e.g., analogous to vector-A in FIGS. 6), and (4) a linearly increasing current using the right side and left side therapy electrodes (e.g., analogous to vector-C in FIG. 6). In implementations, the sequence may be generated in the opposite direction (e.g., counterclockwise instead of clockwise around the patient's torso). In implementations, a defibrillation controller may generate different and/or additional types of voltage and current waveforms using pulse width modulation as part of providing defibrillation shocks to a patient. For instance, a defibrillation controller may generate synchronized multiple capacitor exponential decay curves using pulse width modulation.

In FIGS. 14A-14C, the time scale of the defibrillation pulses shown is in ms. As such, the defibrillation pulses may be shaped using pulse width modulation techniques discussed above, which vary the pulses on the us scale, and which integrate along the ms scale to form smoother defibrillation pulse waveforms as shown. In implementations, and as discussed above, the vector sweeping defibrillation pulses may be delivered using electrode placements offset diagonally from a transverse plane bisecting the patient. As an illustration, FIG. 14D shows a diagram 1406 of a patient with four electrodes, which form vectors A, B, C, D, -A, -B, -C, and -D, similar to FIG. 6 described above. However, in addition to the illustration shown in FIG. 6, the electrodes 1-4 are positioned on the patient along a diagonal plane, as shown in a top-down diagram of the patient 1408. Accordingly, while the defibrillation controller may provide vector sweeping shocks using square waves shaped using pulse width modulation techniques as shown in the defibrillation scheme 1410, which is further similar to FIG. 6 above and also according to FIG. 14A discussed above, the vectors A, B, D, -A, -B, and -D go through the heart at a diagonal.

FIG. 15A illustrates an example circuit diagram 1500 for therapy electrodes implementing vector sweeping defibrillation shocks delivered to a patient 1502. The example circuit diagram 1500 includes circuitry for a first therapy electrode 1504a, a second therapy electrode 1504b, a third therapy electrode 1504c, and a fourth therapy electrode 1504d (collectively, therapy electrode circuitry 1504). The therapy electrode circuitry 1504 may include circuitry to charge one or more capacitors in preparation for delivering one or more therapeutic shocks, generate the therapeutic shocks, and deliver the therapeutic shocks to the patient 1502. Accordingly, in implementations, the therapy electrode circuitry 1504 for each associated therapy electrode includes a connection 1506 to one or more defibrillation capacitors. The therapy electrode circuitry 1504 may also include a connection 1508 to high-voltage (HV) capacitor charging circuitry, such as high-voltage capacitor charging circuitry located in a defibrillation controller. As such, in implementations, the one or more defibrillation capacitors associated with the therapy electrode circuitry 1504 may be configured to be charged via energy received from the HV capacitor charging circuitry connection 1508 and transmitted to the defibrillation capacitor(s) connection 1506. In implementations, when the cardiac defibrillator is preparing to deliver vector sweeping therapeutic shocks to the patient 1502, the HV capacitor charging circuitry may selectively connect to the therapy electrode circuitry 1504a, 1504b, 1504c, and 1504d via the HV capacitor charging circuitry connections 1508 to ensure that the associated defibrillation capacitors are charged uniformly or mostly uniformly. When the defibrillation capacitors are ready to deliver the therapeutic shocks, the HV capacitor charging circuitry connections 1508 may be disabled (e.g., the HV capacitor charging circuitry may be turned off).

Once the therapy electrode circuit 1504 is ready to deliver a therapeutic shock to the patient 1502 (e.g., as part of the vector sweeping therapeutic shocks), a gate driver 1510 and a series of switches 1512 may administer a therapeutic shock using the energy stored in the one or more associated defibrillation capacitors. The gate driver 1510 and associated circuitry may be configured to generate voltage for the therapeutic shock. Additionally, in the example circuit diagram 1500, the therapy electrode circuitry 1504 for each therapy electrode may include three HV electronically controlled switches 1512. Two of the switches 1512 are top side switches for single fault safety, which connect the one or more defibrillation capacitors to the respective therapy electrode. The last switch 1512 is a single low side switch. The gate driver 1510 and the switches 1512 also provide the therapeutic shock to an electrode surface 1514.

In this way, the therapy delivery circuit of each respective therapy electrode may include a first portion of bridge circuitry, such as an H-bridge, configured to administer the delivery of the successive therapeutic shocks (e.g., the gate driver 1510 and switches 1512). The second portion of the bridge circuitry is located in another electrode of the cardiac defibrillator electrically connected to the respective therapy electrode. For example, in implementations, the HV capacitor charging circuitry may be located in a defibrillation controller and/or a signal processing node of the cardiac defibrillator.

In implementations, the therapy electrode circuitry 1504 may further include circuitry configured to isolate each respective therapy electrode during the delivery of the vector sweeping defibrillation shocks when the respective therapy electrode is not delivering a therapeutic shock. As an illustration, the therapy electrode circuitry 1504 may include a shunt circuit configured to isolate the respective therapy electrode, for example, including a switchable diode. As another illustration, the therapy electrode circuitry 1504 may include a high-voltage electronically controlled switching device configured to isolate the respective therapy electrode. For example, the high-voltage electronically controlled switching device may include an IGBT.

Additionally, in implementations, a defibrillation controller may implement these electronic gates, switches, and/or the like to perform pulse width modulation as part of shaping voltage waveforms and current waveforms delivered to a patient during defibrillation shocks. For instance, a defibrillation controller may activate electronic gates, switches, and/or the like to control the duty cycle of a capacitor being discharged to deliver defibrillation shocks to a patient according to a duty cycle waveform. Examples of such duty cycle waveforms are shown and described above with reference to FIG. 9 above. As an illustration of the foregoing, using an IGBT to shape voltage waveforms using pulse width modulation is discussed above with respect to FIGS. 9-13.

In implementations, pulse-modulated defibrillation waveforms can be created through a defibrillation controller generating a pulse width modulation (PWM) signal and using the PWM signal to control switching elements in the delivery system, such as in an H-bridge and/or its isolation switch. Examples of switching elements include a metal-oxide-semiconductor-field-effect transistors (MOSFETs), IGBTs, silicon carbide (SiC) MOSFETs, and/or the like. For instance, the switching element may be a fast-switching IGBT. As noted above, the defibrillation controller can rapidly turn these switching elements on and off to modulate the current through the patient as one or more capacitors are discharged. In implementations, alternatively or additionally, a defibrillation controller may similarly modulate the frequency of defibrillation waveforms using pulse frequency modulation (PFM) or use a combination of varying pulse train frequency and duty cycle (e.g., using a combination of PWM and PFM). In implementations, the defibrillation controller may use open-loop or closed-loop controls. Regardless, the control loop may be based on the peak voltage and/or current and adjusted to provide smooth transitions from vector to vector. While topologies may vary, the defibrillation controller may control the switching element(s) in the energy delivery subsystem via a PWM (and/or, in implementations, a PFM) signal. For example, the defibrillation controller may include therapy delivery circuitry configured as or similarly to the therapy delivery circuit 268 discussed above, which includes the switching element(s) and is configured to generate the PWM (and/or PFM) signal. The therapy delivery circuitry may generate the PWM signal using a microprocessor in software and/or a hardware in which a subcircuit generates the PWM signal from an analog voltage.

As an illustration, including PWM functionality in a cardiac defibrillator requires generating a PWM output. This PWM output is used to control at least one switch in the defibrillation controller of the cardiac defibrillator. These switches much be “fast,” such that the gate impedance allows for fast switching of the defibrillation controller. In implementations, the fast switching may be able to switch on and off on a microsecond or less scale, for example, as shown in FIG. 8. For example, the switch may be able to switch on and within a 0.01 μs, within a 0.1 μs, within 1.0 μs, and/or the like. To minimize self-heating, these switches should be driven from gate drivers capable of very fast rise times. In implementations, for instance, the top or bottom of a bridge is energized in a specific order to pulse the current through various vectors. The bridges are tied together so that at any given time, any therapy electrode can be either sourcing pulse current from its respective capacitor or sinking the current to a common ground. During PWM, the top and/or bottom sides of the bridge(s) are rapidly signaled to turn on or off as needed to deliver faster current pulses.

FIGS. 15B-15E illustrate another example circuit diagram 1520 that may be implemented in a defibrillator controller of a cardiac defibrillator. For example, the example circuit diagram 1520 may be implemented in the defibrillation controller 214, the defibrillation controller 250, and/or the defibrillation controller for any of the cardiac defibrillators discussed with reference to FIGS. 19-21 below. The circuit diagram 1520 includes an AND Gate 1522 configured to perform an operation based on its inputs. Overall, a PWM_SIG 1526 is used to toggle an IGBT (e.g., IGBT_STD) on and off through a gate control 1521.

More specifically, AND Gate 1522 is controlled by a TOPSW_CTL input 1524, which defines the overall envelope for the phases of a defibrillation shock, and the PWM_SIG 1526, which is the PWM signal used to modulate the defibrillation waveform. When the phases are enabled and firing, they will be modulated according to the PWM duty cycle of the PWM_SIG signal 1526. For example, the PWM_SIG signal 1526 may instruct the AND Gate 1522 when to turn on and off to modulate the pulses from one or more discharging capacitors. Thus, the duty cycle of the pulses can be controlled according to the desired defibrillation waveforms for the defibrillation shock. In implementations, other circuits may be used to achieve pulse wave modulated waveforms. For example, some control schemes may require multiple PWM signals and/or different drive methodologies. As an illustration, a defibrillation controller may use low-side switching as another drive methodology.

In implementations, feedback of the pulse voltage can be provided to a processor 1528, for example, through a PULSE_VOLTAGE input 1530. The processor 1528 can then adjust the pulses to provide either the full bandwidth PWM signal or a smoothed version of the PWM signal as would be experienced by the patient's body/cardiomyocytes. One advantage of using real-time feedback to dynamically adjust the PWM signal is that the patient's transthoracic impedance would not necessarily need to be known prior to delivering the defibrillation pulses.

Alternatively, or additionally, in implementations, a defibrillation controller may be configured to modulate vector sweeping defibrillation waveforms to compensate for differences in tissue impedance along the vector sweeping path. For example, such compensation may thereby provide an approximately constant level of vector sweeping defibrillation current radially along the vector sweeping path. As an illustration, a defibrillation controller may take tissue impedance measurements along a vector sweeping path. Such tissue impedance measurements may be determined, for instance, using falloff signals facilitated by a driven ground electrode discussed above with reference to FIG. 1A. Accordingly, a defibrillation controller may use pulse width modulation to adjust for tissue impedance measurements by to provide higher voltage in areas of greater impedance to maintain the desired level of current around the patient's torso. For example, a defibrillation controller could adjust the pulse wave modulation for defibrillation waveforms to obtain a specific wave shape according to a predetermined table of PWM duty cycles. To illustrate, these predetermined PWM duty cycle values could be stored in a software look-up table. In implementations, a defibrillation controller may take impedance measurements during the delivery of each therapeutic pulse and uses the measured impedance as an input to update the duty cycle of the next pulse. This process may help the defibrillation controller achieve the target current.

As another illustration, a defibrillation controller may receive one or more physical body measurements for the patient. For example, the defibrillation controller may receive one or more physical body measurements for the patient from a patient caregiver via an input device of the defibrillation controller or operably connected to the defibrillation controller. With reference to the defibrillation controller 250, for instance, the defibrillation controller 250 may receive one or more physical body measurements for the patient via the user interface 260. Example physical body measurements may include BMI, a waist measurement, a hip measurement, a waist-to-hip measurement, a measurement around a transverse place intersecting the bottom of the patient's sternum, and/or the like. The defibrillation controller may use the one or more physical body measurements for the patient to determine the patient's likely impedance around their torso. For instance, the defibrillation controller may compare the one or more physical body measurements against stored sample physiology models with corresponding stored impedances to determine the patient's likely impedance around their torso. The defibrillation controller may then use pulse width modulation to adjust for these tissue impedance measurements, such as by providing higher voltage in areas of greater impedance as discussed above.

In implementations, a defibrillation controller may be configured to adjust the defibrillation waveforms to be delivered to the patient as part of a vector sweeping shock based on determining whether each of the therapy electrodes are still attached to the patient. For example, a defibrillation controller may inject falloff signals into a patient using a driven ground electrode as discussed above. The defibrillation controller may then detect these falloff signals at each of the therapy electrodes and determine, using the falloff signals, whether each of the therapy electrodes has a sufficient level of attachment to the patient. For example, the defibrillation controller may determine whether each therapy electrode is detecting at least a minimum amplitude of falloff signal. As an illustration, the defibrillation controller may determine whether each therapy electrode is detecting at least 20%, 30%, 40%, 50%, and/or the like of the amplitude of the injected falloff signal.

If a given therapy electrode is not detecting at least a minimum amplitude of falloff signal, the defibrillation controller may determine that the given therapy electrode lacks a sufficient level of attachment to the patient. Upon determining that at least one therapy electrode lacks a sufficient level of attachment to the patient, the defibrillation controller may adjust the defibrillation waveforms to be delivered to a patient to compensate for the at least one therapy electrode that has fallen off. For instance, such compensation may be based on the predetermined locations of the therapy electrodes still having a sufficient level of attachment to the patient. As an example, the defibrillation controller may adjust one or more voltage waveforms to be administered to the patient by the therapy electrodes with the sufficient level of attachment. Example adjustments may include adjusting a timing of the voltage waveforms to compensate for the missing therapy electrode, adjusting shape of the voltage waveforms using pulse width modulation to approximate the desired level or levels of current around the patient's torso, and/or the like. As another example, the defibrillation controller may switch the delivery of the vector sweeping defibrillation shocks to multi-sequential defibrillation shocks to be administered by the therapy electrodes with the sufficient level of attachment. To illustrate, the defibrillation controller may switch to multi-sequential defibrillation shocks in response to determining that not enough therapy electrodes are sufficiently attached to the patient to provide the desired level or levels of current around the patient's torso using pulse width modulation. To illustrate another example, the defibrillation controller may switch to multi-sequential defibrillation shocks as a backup whenever the defibrillation controller determines that a certain number of therapy electrodes lack the sufficient level of attachment. For instance, the defibrillation controller may switch to multi-sequential defibrillation shocks when the defibrillation controller determines that one or more therapy electrodes have fallen off, that two or more therapy electrodes have fallen off, and/or the like. In examples, the multi-sequential defibrillation shocks may be provided as truncated exponential waveforms.

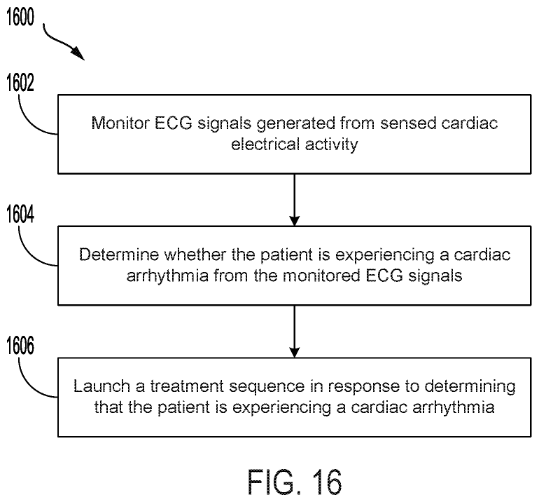

FIG. 16 illustrates a sample process flow for providing vector sweeping therapeutic shocks to a patient. For example, the sample process 1600 shown in FIG. 16 can be implemented by a defibrillation controller of a cardiac defibrillator, such as the defibrillation controller 214 of the wearable cardiac defibrillator 200 or a defibrillation controller of any of the embodiments of a cardiac defibrillator discussed below with reference to FIGS. 19-21. As shown in FIG. 16, the defibrillation controller monitors ECG signals generated from sensed cardiac electrical activity at step 1602. In implementations, the cardiac defibrillator generates ECG signals as discussed above with reference to ECG electrodes 204 shown in FIGS. 1A and 1B. For instance, the cardiac defibrillator may include ECG electrodes (e.g., configured as or configured similarly to the ECG electrodes 204) configured to sense cardiac electrical activity of the patient and generate ECG signals based on the sensed cardiac electrical activity. The ECG electrodes, a signal processing node (e.g., configured as or configured similarly to the signal processing node 210), and/or the defibrillation controller may amplify, filter, and/or digitize the ECG signals, which the defibrillation controller uses to monitor the patient for cardiac arrhythmias.

The defibrillation controller determines whether the patient is experiencing a cardiac arrhythmia from the monitored ECG signals at step 1604. In implementations, the defibrillation controller may include a cardiac event detector (e.g., configured as or configured similarly to the cardiac event detector 264) configured to detect whether the patient is experiencing a cardiac arrhythmia using the ECG signals, as described above with reference to the cardiac event detector 264. As an example, the cardiac event detector may detect cardiac arrhythmias in the ECG signals using heart rate and/or cardiac cycle morphology.

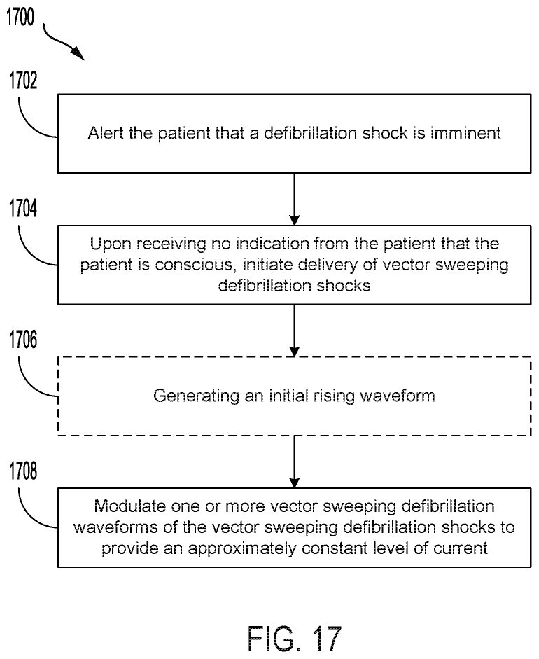

The defibrillation controller launches a treatment sequence in response to determining that the patient is experiencing a cardiac arrhythmia at step 1606. To illustrate, the treatment sequence may include alerting the patient and/or bystanders of an impending defibrillation shock, waiting for a patient response to the alert, preparing a vector sweeping defibrillation shock, and/or delivering the vector sweeping defibrillation shock. In implementations, the treatment sequence proceeds according to the process flow shown in FIG. 17. As such, the defibrillation controller of a cardiac defibrillator, such as the defibrillation controller 214 of the wearable cardiac defibrillator 200 or a defibrillation controller of any of the embodiments of a cardiac defibrillator discussed below with reference to FIGS. 19-21, may implement sample process 1700 of FIG. 17.

The defibrillation controller alerts the patient that a defibrillation shock is imminent at step 1702. In implementations, the defibrillation controller may include an alarm manager (e.g., configured as or similarly to the alarm manager 266 shown in FIG. 1B). The defibrillation controller may thus issue alerts to the patient as described above with reference to FIG. 1B. For example, alerts may be visual, auditory, and/or tactile. In examples, the alerts may also include instructions to the patient that the patient can abort the delivery of the defibrillating shock by interacting with a user interface. The user interface may include, in implementations, one or more response buttons that the patient can press to indicate to the defibrillation controller that the patient is still conscious.

The defibrillation controller, upon receiving no indication from the patient that the patient is conscious, initiates delivery of vector sweeping defibrillation shocks. In implementations, the defibrillation controller initiates delivery of vector sweeping defibrillation shocks, via therapy electrodes of the cardiac defibrillator, along a vector sweeping path around the patient's torso. The therapy electrodes may be configured according to the therapy electrodes 206 described with reference to the wearable cardiac defibrillator 200 of FIGS. 1A and 1B above and/or any of the therapy electrodes described below in the context of the cardiac defibrillators of FIGS. 19-21. Additionally, in implementations, the therapy electrodes may be configured around the body as shown in any of FIGS. 2-5H to create the vector sweeping path.

In implementations, initiating the delivery of vector sweeping defibrillation shocks may include, for example, charging one or more capacitors used to deliver the vector sweeping defibrillation shocks and then discharging the one or more capacitors to deliver a first waveform or waveforms of the vector sweeping defibrillation shocks. As an illustration, a defibrillation controller may include a therapy delivery circuit (e.g., configured as or similarly to the therapy delivery circuit 268 described with reference to FIG. 1B), which includes one or more capacitors. The therapy delivery circuit may charge the capacitors from a power source (e.g., configured as or similarly to the battery 262) as described above with reference to FIG. 1B. In examples, the defibrillation controller may charge the capacitors at least partially while delivering the alert to the patient at step 1702.

In implementations, the defibrillation controller may generate an initial rising waveform at step 1706. For instance, the defibrillation controller may generate an initial rising waveform as a ramp as shown and described above with reference to FIGS. 12 and 13 above. In examples, the defibrillation controller may generate the initial rising waveform before modulating the one or more vector sweeping defibrillation waveforms of the vector sweeping defibrillation shocks at step 1708, described below. As an illustration, the defibrillation controller may generate the initial rising waveform as part of initiating the delivery of the vector sweeping defibrillation shocks at step 1704. For example, the defibrillation controller may generate the initial rising waveform as described above with reference to FIG. 12 before the beginning of the vector sweeping therapeutic shocks, such that the ramp occurs before the rest of the pulse width modulation. In examples, the defibrillation controller may generate the initial rising waveform as part of modulating the one or more vector sweeping defibrillation waveforms of the vector sweeping defibrillation shocks (e.g., at step 1708). As an example, the defibrillation controller may generate the initial rising waveform during the vector as described above with reference to FIG. 13. In implementations, the defibrillation controller may not generate an initial rising waveform, which is why step 1706 is marked with dotted lines.

The defibrillation controller modulates one or more vector sweeping defibrillation waveforms of the vector sweeping defibrillation shocks to provide an approximately constant level of current. For example, in implementation, the defibrillation controller modulates the one or more vector sweeping defibrillation waveforms to provide the approximately constant level of vector sweeping current radially along a vector sweeping path. In implementations, the defibrillation controller is configured to modulate the vector sweeping defibrillation waveforms as described above with reference to FIGS. 9-14C above. Such modulations may be carried out, for example, by the therapy delivery circuit of the defibrillation controller. As an illustration, the defibrillation controller may modulate a duty cycle waveform used to discharge one or more capacitors of the defibrillation controller, and thereby modulate the therapy electrode voltage waveforms delivered by the therapy electrodes, to provide the approximately constant level of current. Such modulation may occur, for instance, according to any of FIGS. 9-13 above. In examples, modulating the one or more vector sweeping defibrillation waveforms of the vector sweeping defibrillation shock includes modulating a therapy electrode voltage for each of the therapy electrodes according to an approximate sine wave, as described above with respect to FIGS. 9-13 above.

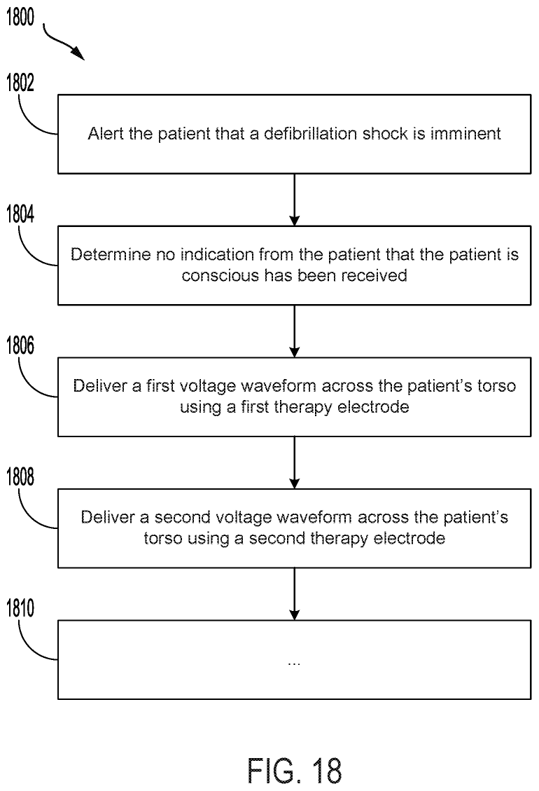

In implementations, the treatment sequence proceeds according to the process flow shown in FIG. 18. As such, the defibrillation controller of a cardiac defibrillator, such as the defibrillation controller 214 of the wearable cardiac defibrillator 200 or a defibrillation controller of any of the embodiments of a cardiac defibrillator discussed below with reference to FIGS. 19-21, may implement sample process 1800 of FIG. 18. The defibrillation controller alerts the patient that a defibrillation shock is imminent at step 1802. In implementations, the defibrillation controller may implement step 1802 similarly to step 1702, as discussed above.

The defibrillation controller determines no indication from the patient that the patient is conscious has been received at step 1804. In implementations, the patient may be able to indicate that the patient is conscious by interacting with a user interface. Examples of a user interface include one or more response buttons provided on the defibrillation controller, on a separate patient interface pod (e.g., configured as or similarly to the patient interface pod 212 shown in FIG. 1A), a button provided on a personal user device, and/or the like. To illustrate, the defibrillation controller may transmit the alert to a personal user device associated with the patient, such as a smartphone. The patient may interact with a graphical user interface displayed on the personal user device, where the graphical user interface includes one or more response buttons. In implementations, as discussed above with respect to FIG. 1A, if the defibrillation controller determines that the patient has interacted with the user interface, the defibrillation controller may delay or stop the delivery of the defibrillation shocks. By contrast, if the defibrillation controller determines that the patient has not interacted with the user interface during a predetermined time period, the defibrillation controller may proceed to delivering the defibrillation shocks. For example, a predetermined time period may last while the defibrillation controller is providing alerts or alarms to the patient at step 1802. If the patient does not interact with the user interface during the alert time period, the defibrillation controller may determine that no indication from the patient that the patient is conscious has been received.

The defibrillation controller delivers a first voltage waveform across the patient's torso using a first therapy electrode at step 1806. The defibrillation controller delivers a second voltage waveform across the patient's torso using a second therapy electrode at step 1808. In implementations, the defibrillation controller is configured to deliver the second voltage waveform at least partially simultaneously with the first voltage waveform. In various embodiments, the defibrillation controller may deliver additional voltage waveforms using the same and/or different therapy electrodes, as represented by step 1810. In implementations, the defibrillation controller may deliver the at least first and second waveforms as shown and described with respect to any of FIGS. 9-14C above. In implementations, the delivery of the at least first voltage waveform and second voltage waveform cause an approximately level of vector sweeping defibrillation current to be delivered radially around the patient's torso, as discussed with respect to FIGS. 9-13 above.

In implementations, a cardiac defibrillator may use a modified process flow compared to the sample processes 1600, 1700, and/or 1800. For example, in various embodiments, a cardiac defibrillator may be configured as an AED. Some embodiments of an AED may not include ECG sensing electrodes. As such, implementations of a cardiac defibrillator applying sample process 1600 may not perform steps 1602 and 1604 and instead may only launch the treatment sequence according to step 1606. To illustrate, an AED may launch the treatment sequence in response to a healthcare professional indicating to the AED that the AED should apply defibrillation shocks to the patient, such as by interacting with a user interface of the AED.

Alternatively, or additionally, implementations of an AED may not be configured to alert a patient that a defibrillation shock is imminent, or any similar alerts may be configured differently. For example, an AED may be configured to alert a patient and any bystanders that a defibrillation shock will be presently delivered, but unlike embodiments of the wearable cardiac defibrillator discussed above and below, the patient may have no ability to interact with the AED to indicate to the AED that the patient is still conscious. Accordingly, implementations of a cardiac defibrillator applying sample process 1700 may not include step 1702 and may apply step 1704 simply in response to the cardiac defibrillator launching the treatment sequence. Alternatively, implementations of a cardiac defibrillator applying sample process 1700 may include 1702 but may apply step 1704, not in response to whether or not an indication has been received from the patient as to whether the patient is conscious, but as part of the scheduled part of the treatment sequence. As another example, the cardiac defibrillator may be able to receive an indication to delay or abort the treatment sequence, but such an input may be made using a user input of the cardiac defibrillator in a way that may or may not be related to the patient's state of consciousness. For instance, an AED may include a button that allows a first responder to abort the treatment sequence for any reason. Along these same lines, implementations of a cardiac defibrillator applying sample process 1800 may not include steps 1802 and/or 1804.

In implementations, a cardiac defibrillator configured to provide vector sweeping therapeutic shocks to a patient may also be configured to provide other types of therapy to the patient, such as pacing therapy. As an illustration, at least some of the therapy electrodes may be configured to also generate and deliver pacing pulses to the patient. Accordingly, the circuitry configured to generate and deliver the vector sweeping therapeutic shocks may also be configured for pacing. For example, a given therapy electrode may deliver a pacing pulse to the patient using stored energy in one or more defibrillation capacitors associated with the therapy electrode. In implementations, the cardiac defibrillator may deliver pacing pulses to the patient's heart using a similar process as delivering the vector sweeping defibrillation shocks, except at a lower energy level. For example, the cardiac defibrillator may deliver vector sweeping defibrillation shocks in a predetermined sequence along a vector sweeping path around the patient's torso. The vector sweeping path and/or vectors used for vector sweeping defibrillation shocks may be the same as or different from the vector sweeping path and/or vectors used to deliver pacing pulses. For example, cardiac defibrillator may use a first predetermined rotational series of therapy vectors for defibrillation shocks and a second predetermined rotational series of therapy vectors for pacing shocks.

In implementations, the cardiac defibrillator may be configured to deliver the pacing pulses synchronously with the patient's cardiac cycle. As such, the defibrillation controller may be configured to determine the patient's cardiac cycle using current or recent ECG cycles of the patient (e.g., based on the last 5 cardiac cycles, based on the last 10 cardiac cycles, based on the last 20 cardiac cycles, etc. of the patient). The defibrillation controller may then control the delivery of the pacing pulses to occur at a certain point in the patient's cardiac rhythm. For example, the defibrillation controller may use a certain number of previous cardiac cycles (e.g., 5, 10, 15, 20, etc.) to determine an average cardiac cycle length, such as an average R-R interval. The defibrillation controller may then control the delivery of the pacing pulses to pace the patient, for example, according to a certain portion of the patient's cardiac cycle based on the previous cycles and average cardiac cycle length. For instance, the defibrillation controller may pace the patient according to when the cardiac controller predicts the patient's next Q wave will occur. In implementations, the cardiac defibrillator may use a feedback loop in delivering pacing pulses to the patient. For example, the cardiac defibrillator may use ECG signals and/or cardiovibrational signals to update subsequent shock timings, energy levels, etc. based on whether previous pacing pulses succeeded or failed. As another example, the cardiac defibrillator may use impedance measurements to adjust energy levels of future pacing pulses to maintain a predetermined current level during pacing.

Implementing pacing using therapy vectors as described above may have several advantages over other systems for external pacing. When pacing a patient's heart, it may be difficult to determine capture, or whether the shock succeeds or fails at inducing a heartbeat. With pacing using vector sweeping shocks, the cardiac defibrillator may be able to deliver lower energy pulses compared to other external pacing systems. In addition to such lower energy pulses being more tolerable for a patient, lower energy pulses may make it easier for the cardiac defibrillator to determine capture. For example, the electrode components sensing the patient's surface electrical activity may be less likely to saturate, making it easier for the cardiac defibrillator to determine whether the pacing pulse succeeded at inducing a heartbeat. Similar considerations may apply to a cardiovibrational sensor (e.g., because the lower energy pulse may mean that the patient moves less during a pacing pulse). In examples, the ECG electrodes may be sequentially isolated during pacing pulses in time with the therapy vectors to prevent saturation of the ECG electrodes closest to the therapy electrodes while allowing other sensing electrode components to continue to sense surface electrical activity.

As discussed above, the wearable cardiac defibrillator 200 shown in FIGS. 1A and 1B is an example of a wearable cardiac defibrillator that can be modified or otherwise configured to provide the vector sweeping defibrillation shocks described herein. Referring back to the defibrillation controller 250 shown in FIG. 1B, the defibrillation controller 250 may include additional functionalities that the defibrillation controller 250 uses to monitor the patient's cardiac status and deliver treatment. For example, with reference to the sensor interface 254, the sensor interface 254 may be electrically connected to additional types of sensors, as noted above. As another illustration, the one or more cardiovibration sensors 274 can be configured to detect cardiac or pulmonary vibration information. The one or more cardiovibration sensors 274 can transmit information descriptive of the cardiovibrations (and other types of sensed vibrations) to the sensor interface 254 for subsequent analysis. For example, the one or more cardiovibration sensors 274 can detect the patient's heart valve vibration information (e.g., from opening and closing during cardiac cycles). As a further example, the one or more cardiovibration sensors 274 can be configured to detect cardiovibrational signal values including one or more of S1, S2, S3, and/or S4 cardiovibrational biomarkers. From these cardiovibrational signal values or heart vibration values, certain heart vibration metrics may be calculated (e.g., at the wearable cardiac defibrillator 200 and/or at a remote server). These heart vibration metrics may include one or more of electromechanical activation time (EMAT), average EMAT, percentage of EMAT (% EMAT), systolic dysfunction index (SDI), and/or left ventricular systolic time (LVST). In implementations, the cardiovibrational signal values from the one or more cardiovibration sensors 274 can be used to detect whether the patient is experiencing a cardiac arrhythmia.

The one or more cardiovibration sensors 274 can also be configured to detect heart wall motion, for instance, by placement of the sensor in the region of the apical beat. In implementations, the one or more cardiovibration sensors 274 can include a vibrational sensor configured to detect vibrations from the patient's cardiac and pulmonary system and provide an output signal responsive to the detected vibrations of a targeted organ. For example, the one or more cardiovibration sensors 274 may be configured to detect vibrations generated in the trachea or lungs due to the flow of air during breathing. In implementations, additional physiological information can be determined from pulmonary-vibrational signals such as, for example, lung vibration characteristics based on sounds produced within the lungs (e.g., stridor, crackle, etc.). In implementations, the one or more cardiovibration sensors 274 can include a multi-channel accelerometer, for example, a three-channel accelerometer configured to sense movement in each of three orthogonal axes such that patient movement/body position can be detected and correlated to detected cardiovibration information.

As another illustration, the tissue fluid monitor 276 may include a radiofrequency (RF) sensor configured to take bio-impedance measurements of the patient's thorax. In implementations, sensor interface 254 may use the bio-impedance measurements to determine a thoracic fluid level in the patient 202. An example embodiment of the tissue fluid monitor 276 includes at least one RF antenna, such as a transmitting antenna and a receiving antenna, or a single antenna configured to transmit and receive RF waves. The tissue fluid monitor 276 also includes RF circuitry (e.g., implemented by the sensor interface 254) configured to transmit a low-power signal in an ultra-high frequency band (e.g., 0.1 GHz to 5.0 GHz, 0.5 GHz to 2.1 GHZ) at a predetermined rate (e.g., every 10 ms, every 20 ms, every 30 ms, every 40 ms, every 50 ms, etc.). The tissue fluid monitor 276 receives RF-based biosignals indicative of the thoracic fluid level in the patient 202 in the form of RF waves transmitted through the patient 202, scattered by the patient 202, and/or reflected from the patient 202. For example, the tissue fluid monitor 276 may detect transmitted, scattered, and/or reflected RF waves for a predetermined amount of time (e.g., about 30 seconds, about 1 minute, about 2 minutes, about 3 minutes, about 5 minutes, about 10 minutes, etc.).

In implementations, the wearable cardiac defibrillator 200 is configured to gate when RF measurements are taken and/or discard certain RF measurements based on the patient's state when the RF measurements were taken. For example, the sensor interface 254 may determine whether the patient 202 showed movement above a predetermined threshold before tissue fluid monitor 276 started the RF measurements process and/or while the RF measurements were taking place. If RF measurements were taken during or immediately after movement above the predetermined threshold, the sensor interface 254 may discard those RF measurements. In implementations, at least some of the analysis and/or gating of RF measurements may be performed by a remote server. As an example, the defibrillation controller 250 may transmit the RF measurements to the remote server via the network interface 258 and receive back from the remote server a determined thoracic fluid level.

In implementations, the sensor interface 254 may be connected to one or more motion sensors (e.g., one or more accelerometers, gyroscopes, magnetometers, ballistocardiographs, etc.) as part of the externally applied sensors 272. In implementations, the defibrillation controller 250 may include a motion sensor interface 278, either implemented separately or as part of the sensor interface 254. For instance, as shown in FIG. 1B, the defibrillation controller 250 may include a motion sensor interface 278 operatively coupled to one or more motion detectors 280 configured to generate motion data, for example, indicative of physical activity performed by the patient 202 and/or physiological information internal to the patient 202. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. For instance, the motion data may include accelerometer counts indicative of physical activity performed by the patient 202, accelerometer counts indicative of respiration rate of the patient 202, accelerometer orientation indicative of posture information for the patient 202, accelerometer counts indicative of cardiovibrational information for the patient 202, and/or the like.

The motion sensor interface 278 is configured to receive one or more outputs from the motion sensor(s) 280. The motion sensor interface 278 can be further configured to condition the output signals by, for example, converting analog signals to digital signals (if using an analog motion sensor), filtering the output signals, and/or combining the output signals into a combined directional signal. As an illustration, the motion sensor interface 278 may combine each x-axis signal into a composite x-axis signal, combining each y-axis signal into a composite y-axis signal, and combining each z-axis signal into a composite z-axis signal. In examples, the motion sensor interface 278 can be configured to filter the signals using a high-pass or band-pass filter to isolate the acceleration of the patient 202 due to movement from the component of the acceleration due to gravity. Additionally, the motion sensor interface 278 can configure the outputs from a given motion sensor 280 for further processing. For example, the motion sensor interface 278 can be configured to arrange the output of an individual motion sensor 280 as a vector expressing acceleration components of the x-axis, the y-axis, and the z-axis of the motion sensor 280. The motion sensor interface 278 can thus be operably coupled to the processor 270 and configured to transfer the output and/or processed motion signals from the motion sensor(s) 280 to the processor 270 for further processing and analysis.

In implementations, the one or more motion detectors 280 can be integrated into one or more components of the wearable cardiac defibrillator 200, either within the defibrillation controller 250 or external to the defibrillation controller 250, as shown in FIG. 1B. For instance, in some implementations, the one or more motion detectors 280 may be located in or near the ECG electrodes 204. In some implementations, the one or more motion detectors 280 may be located elsewhere on the wearable cardiac defibrillator 200. For example, a motion detector 280 may be integrated into the defibrillation controller 250 (e.g., such that the one or more motion detectors 280 would be located within the housing 252 of the defibrillation controller 250, as shown in FIG. 1B). In some implementations, a motion detector 280 may be integrated into another component of the wearable cardiac defibrillator 200, such as a therapy electrode 206, the signal processing node 210, and/or the like. In some implementations, a motion detector 280 can be integrated into an adhesive ECG sensing and/or therapy electrode patch.

The wearable cardiac defibrillator 200 shown in FIGS. 1A and 1B is also an example of a wearable cardiac defibrillator. Other embodiments of a wearable cardiac defibrillator may similarly be modified or otherwise configured for vector sweeping therapy. For example, FIG. 19 illustrates another embodiment of a wearable cardiac defibrillator that can be modified as discussed above to provide vector sweeping defibrillation shocks to a patient. More specifically, FIG. 19 shows a hospital wearable cardiac defibrillator 1900 that is external, ambulatory, and wearable by a patient 1901. In some implementations, the hospital wearable cardiac defibrillator 1900 can be configured to also provide pacing therapy, e.g., to treat bradycardia, tachycardia, and asystole conditions. In examples, the patient 1901 may use the hospital wearable cardiac defibrillator 1900 in hospital settings (e.g., for cardiac protection after a cardiac event). In examples, the patient 1901 may use the hospital wearable defibrillator in other settings where the patient 1901 is ambulatory (e.g., at the patient's home and workplace).

The hospital wearable cardiac defibrillator 1900 can include one or more ECG electrodes 1902a, 1902b, 1902c (collectively, ECG electrodes 1902), therapy electrodes 1904a and 1904b (collectively, therapy electrodes 1904), and a defibrillation controller 1906. In implementations, the hospital wearable cardiac defibrillator 1900 may also include a signal processing unit 1608. In examples, each of these components can function similarly to the like components of the wearable cardiac defibrillator 200 discussed above with reference to FIGS. 1A and 1B. In implementations, the electrodes 1902 and 1904 can be supported by disposable adhesive patches configured to be removably attached to a skin surface of the patient 1901. For example, the electrodes 1902 and 1904 can include ECG sensing and therapy electrodes disposed on separate sensing and therapy electrode adhesive patches. In implementations, both ECG sensing and therapy electrodes can be integrated and disposed on a same electrode adhesive patch that is then attached to the patient 1901.

As an illustration, the front therapy electrode 1904a attaches to the front of the patient's torso to deliver pacing or defibrillating therapy. Similarly, the back therapy electrode 1904b attaches to the back of the patient's torso. Similarly, in an example scenario, at least three ECG adhesively attachable sensing electrodes 1902 can be attached at least above the patient's chest near the right arm (e.g., ECG electrode 1902a), above the patient's chest near the left arm (e.g., ECG electrode 1902b), and towards the bottom of the patient's chest (e.g., ECG electrode 1902c) in a manner prescribed by a trained professional. In implementations, the hospital wearable cardiac defibrillator 1900 may include additional adhesive therapy electrodes 1904. Additionally, or alternatively, the patches shown in FIG. 19 may include additional therapy electrodes 1904 on them. As an illustration, the hospital wearable cardiac defibrillator 1900 may be configured with adhesive therapy electrodes 1904 configured in any of the positions shown in FIGS. 2-5H. In this way, at least two vectors may be formed between the therapy electrodes 1904 of the hospital wearable cardiac defibrillator 1900.

In implementations, the defibrillation controller 1906 may be configured to function similarly to the defibrillation controller 214 of FIG. 1A and defibrillation controller 250 of FIG. 1B. As shown in FIG. 19, the defibrillation controller 1906 may include a user interface 1910 configured to communicate information, for instance, with the patient 1901. In examples, the patient 1901 being monitored by a hospital wearable cardiac defibrillator 1900 may be confined to a hospital bed or room for a significant amount of time (e.g., 75% or more of the patient's stay in the hospital). As a result, a user interface 1910 can be configured to interact with a user other than the patient 1901 (e.g., a technician, a clinician, and/or other caregiver) for device-related functions. Examples of such device-related functions include initial device baselining, setting and adjusting patient parameters, changing the device batteries, and/or charging the device.

The hospital wearable cardiac defibrillator 1900 may be modified to provide the vector sweeping defibrillation shocks described above. For example, in implementations, the hospital wearable cardiac defibrillator 1900 may include adhesive therapy electrodes. The adhesive therapy electrodes may be configured to be disposed at predetermined locations on the patient 1901. Additionally, the hospital wearable cardiac defibrillator 1900 may be configured to provide vector sweeping defibrillation shocks to the patient 1901 as described, for example, with reference to any of FIGS. 9-14C above.

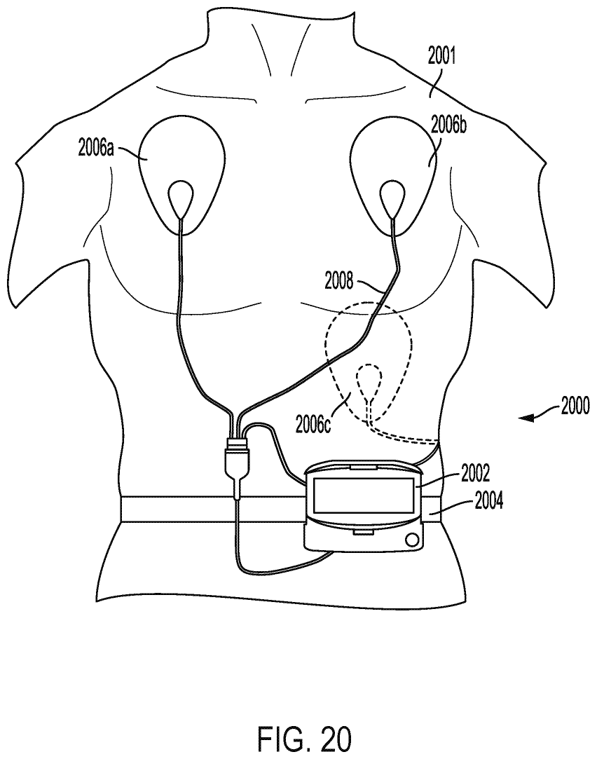

FIG. 20 illustrates another example of a wearable cardiac defibrillator. As shown in FIG. 20, a wearable cardiac defibrillator may include a belted wearable cardiac defibrillator 2000 that is external, ambulatory, and wearable by a patient 2001. In implementations, the belted wearable cardiac defibrillator 2000 may include a defibrillation controller 2002 configured to be worn mounted on a belt 2004 around the patient's torso. In such examples, the belted wearable cardiac defibrillator 2000 may be configured similarly to the hospital wearable defibrillator 1900 shown in FIG. 19. In implementations, the belted wearable cardiac defibrillator 2000 may instead include a defibrillation controller 2002 integrated into the belt 2004. In such implementations, the belt 2004 may include a number of modules housing the circuitry of the defibrillation controller 2002 such that the patient 2001 does not need to wear a separate defibrillation controller 2002. Regardless of the implementation, the defibrillation controller 2002 implemented either as a separate unit or integrated into the belt 2004 may be configured to function similarly to the defibrillation controller embodiments described above.

Similar to the hospital wearable cardiac defibrillator 1900, the belted wearable cardiac defibrillator 2000 can include adhesive patches configured to be removably attached to a skin surface of the patient 2001. In implementations, the adhesive patches are configured to support electrodes 2006a, 2006b, 2006c (collectively, adhesive electrodes 2006). For example, the adhesive electrodes 2006 may be disposable adhesive electrodes in a wired connection 2008 with the defibrillation controller 2002 (or, in implementations, with the belt 2004 including the circuitry of the defibrillation controller 2002). Alternatively, at least some of the adhesive electrodes 2006 may be wirelessly connected to the defibrillation controller 2002 (or, in implementations, with the belt 2004 including the circuitry of the defibrillation controller 2002). For instance, the adhesive electrodes 2006 may be configured to communicate via Bluetooth® with the defibrillation controller 2002. In implementations, at least some of the adhesive electrodes 2006 may include both sensing and therapy electrodes integrated into the same electrode adhesive patch that is attached to the patient 2001. In implementations, at least some of the adhesive electrodes 2006 may be a dedicated ECG electrode or a dedicated therapy electrode. For example, adhesive electrodes 2006a and 2006c may be dedicated therapy electrodes. In implementations, the belted wearable cardiac defibrillator 2000 may include additional adhesive electrodes 2006, which include ECG sensing and/or therapy electrodes configured to form additional therapy electrode vectors.

Similar to the hospital wearable defibrillator 1900 of FIG. 19, the belted wearable cardiac defibrillator 2000 may be modified to provide the vector sweeping defibrillation shocks described above. For example, in implementations and as described, the belted wearable cardiac defibrillator 2000 may include adhesive therapy electrodes. The adhesive therapy electrodes may be configured to be disposed at predetermined locations on the patient 2001. Additionally, the belted wearable cardiac defibrillator 2000 may be configured to provide vector sweeping defibrillation shocks to the patient 2001 as described, for example, with reference to any of FIGS. 9-14C above.

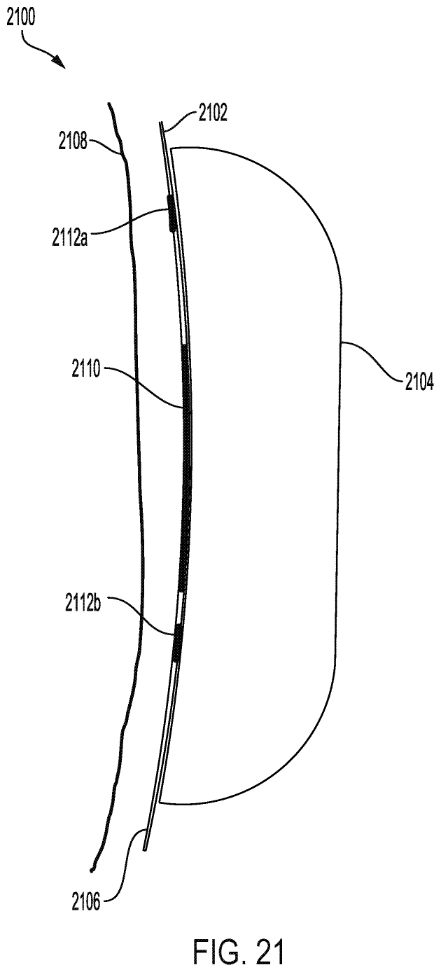

FIG. 21 illustrates another example of a wearable cardiac defibrillator that can be modified to provide vector sweeping therapeutic shocks. As shown in FIG. 21, the wearable cardiac defibrillator may be or may include an adhesive assembly 2100. The adhesive assembly 2100 includes, for example, an adhesive patch configured as a contoured pad 2102 and a housing 2104 configured to form a watertight seal with the contoured pad 2102. In implementations, the housing 2104 is configured to house electronic components of the adhesive assembly 2100. For example, the housing 2104 may house electronic components forming a defibrillation controller (e.g., similar to the defibrillation controller embodiments discussed above, such as with respect to the defibrillation controller 250 of FIG. 1B). The adhesive assembly 2100 includes a conductive adhesive layer 2106 configured to adhere the adhesive assembly 2100 to a skin surface 2108 of a patient. The adhesive layer 2106 may include, for example, a water-vapor permeable conductive adhesive material. Examples of a water-vapor permeable conductive adhesive material include a material selected from the group consisting of an electro-spun polyurethane adhesive, a polymerized microemulsion pressure sensitive adhesive, an organic conductive polymer, an organic semi-conductive conductive polymer, an organic conductive compound, and a semi-organic conductive compound, and/or combinations thereof. As such, the contoured pad 2102 having the adhesive layer 2106 is configured to support the housing 2104 while attached to the patient's body.

The adhesive assembly 2100 also includes at least one therapy electrode 2110 integrated with the contoured pad 2102. In implementations, the adhesive assembly 2100 may include a therapy electrode 2110 that forms a vector with another therapy electrode disposed on another adhesive assembly 2100 adhered to the patient's body and/or with a separate therapy electrode adhered to the patient's body. The adhesive assembly 2100 may also include one or more ECG electrodes 2112 integrated with the contoured pad 2102. For example, as shown, the adhesive assembly 2100 may include ECG electrodes 2112a and 2112b. In implementations, the adhesive assembly 2100 may alternatively or additionally be in electronic communication with a separate ECG electrode, such as a separate adhesive ECG electrode adhered to the patient's body. The electronic communication may be a wired connection or a wireless connection between the adhesive assembly 2100 and the separate ECG electrode. As such, the adhesive layer or layers 2106 of the adhesive assembly or assemblies 2100 are configured to support the ECG electrodes 2112 and the therapy electrodes 2110. In examples, as shown in FIG. 21, the therapy electrode(s) 2110 and ECG electrode(s) 2112 may be formed within the contoured pad 2102 such that a skin-contacting surface of each component is coplanar with or protrudes from the patient-contacting face of the contoured pad 2102.

Similar to the hospital wearable cardiac defibrillator 1900 of FIG. 19 and the belted wearable cardiac defibrillator 2000 of FIG. 20, the adhesive assembly 2100 may be modified to provide the vector sweeping defibrillation shocks described above. As an example, in implementations, the adhesive assembly 2100 may include therapy electrodes 2110 and be electronically connected to one or more additional therapy electrodes, which together are configured to be disposed at predetermined locations on the patient. To illustrate, the additional therapy electrodes may be disposed on one or more separate contoured pads configured to be adhesively attached to the patient. Additionally, the adhesive assembly 2100 may be configured to provide vector sweeping defibrillation shocks to the patient 2001 as described, for example, with reference to any of FIGS. 9-14C above.

These other embodiments of a wearable cardiac defibrillator shown in FIGS. 19-21 may include other features of the wearable cardiac defibrillator 200 of FIG. 1A. For example, in implementations, a hospital wearable cardiac defibrillator 1900, a belted wearable cardiac defibrillator 2000, and/or an adhesive assembly 2100 may include a patient interface unit similar to the patient interface pod 212. Alternatively, or additionally, a hospital wearable cardiac defibrillator 1900, a belted wearable cardiac defibrillator 2000, and/or an adhesive assembly 2100 may include another user interface that the patient using the wearable cardiac defibrillator can use to indicate to the wearable cardiac defibrillator that the patient is still conscious. For example, a hospital wearable cardiac defibrillator 1900, a belted wearable cardiac defibrillator 2000, and/or an adhesive assembly 2100 can include one or more response buttons similar to those described above with respect to the wearable cardiac defibrillator 200.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-204. (canceled)

205. A cardiac defibrillator configured to provide vector sweeping therapeutic shocks to a patient, comprising:
a plurality of electrocardiogram (ECG) electrodes configured to sense cardiac electrical activity of an ambulatory patient;
a plurality of therapy electrodes configured to be positioned at predetermined locations on the patient; and
a defibrillation controller configured to be electrically connected to the plurality of ECG electrodes and the plurality of therapy electrodes, wherein the defibrillation controller is configured to
monitor ECG signals generated from the sensed cardiac electrical activity,
determine whether the patient is experiencing a cardiac arrhythmia from the monitored ECG signals, and
launch a treatment sequence in response to determining that the patient is experiencing a cardiac arrhythmia, wherein the treatment sequence comprises
alerting the patient that a defibrillation shock is imminent;
upon receiving no indication from the patient that the patient is conscious, initiating delivery of vector sweeping defibrillation shocks, via the plurality of therapy electrodes, along a vector sweeping path around the patient's torso; and
modulating one or more vector sweeping defibrillation waveforms of the vector sweeping defibrillation shocks to provide an approximately constant level of vector sweeping defibrillation current radially along the vector sweeping path.

206. The cardiac defibrillator of claim 205, wherein the vector sweeping path comprises a 360 degree path around a vertical axis of the patient's torso.

207. The cardiac defibrillator of claim 205, wherein the approximately constant level of vector sweeping defibrillation current comprises an average current between 3 and 75 Amps.

208. The cardiac defibrillator of claim 207, wherein the approximately constant level of vector sweeping defibrillation current comprises an average current between 5 and 20 Amps.

209. The cardiac defibrillator of claim 205, wherein modulating the one or more vector sweeping defibrillation waveforms of the vector sweeping defibrillation shocks comprises applying pulse width modulation to provide the approximately constant level of vector sweeping defibrillation current radially along the vector sweeping path.

210. The cardiac defibrillator of claim 205, wherein the delivery of the vector sweeping defibrillation shock, via the plurality of therapy electrodes, is along a plurality of vectors across the patient's torso.

211. The cardiac defibrillator of claim 210, wherein the plurality of vectors are configured with fine vector angular resolution and smooth transitions between adjacent vectors.

212. The cardiac defibrillator of claim 210, wherein the plurality of vectors across the patient's torso are defined by a plurality of voltage waveforms administered to the patient by the plurality of therapy electrodes.

213. The cardiac defibrillator of claim 212, wherein the plurality of voltage waveforms comprise a plurality of half sinusoidal waveforms.

214. The cardiac defibrillator of claim 212, where the plurality of voltage waveforms comprise a plurality of synchronized multiple capacitor exponential decay curves.

215. The cardiac defibrillator of claim 205, wherein modulating the one or more vector sweeping defibrillation waveforms of the vector sweeping defibrillation shocks comprises applying pulse width modulation to provide the approximately constant level of vector sweeping defibrillation current radially along the vector sweeping path.

216. The cardiac defibrillator of claim 205, wherein modulating the one or more vector sweeping defibrillation waveforms of the vector sweeping defibrillation shock comprises modulating a therapy electrode voltage for each of the plurality of therapy electrodes according to an approximate sine wave.

217. The cardiac defibrillator of claim 216, wherein the approximate sine waves of the plurality of therapy electrodes are spaced radially even to each other.

218. The cardiac defibrillator of claim 205, wherein the defibrillation controller is further configured to
detect a falloff signal at each of the plurality of therapy electrodes; and
determine, using the falloff signals, whether each of the therapy electrodes has a sufficient level of attachment to the patient.

219. The cardiac defibrillator of claim 218, wherein the defibrillation controller is further configured to adjust the delivery of the vector sweeping defibrillation shocks in response to detecting that one or more of the plurality of therapy electrodes lacks the sufficient level of attachment to the patient based on the falloff signals.

220. The cardiac defibrillator of claim 219, wherein the defibrillation controller is configured to adjust the delivery of the vector sweeping defibrillation shocks based on the predetermined locations on the patient associated with one or more of the plurality of therapy electrodes having the sufficient level of attachment to the patient.

221. The cardiac defibrillator of claim 219, wherein the defibrillation controller is configured to adjust the delivery of the vector sweeping defibrillation shocks by switching the delivery of the vector sweeping defibrillation shocks to multi-sequential defibrillation shocks to be administered by the one or more of the plurality of therapy electrodes having the sufficient level of attachment to the patient.

222. The cardiac defibrillator of claim 205, wherein the cardiac defibrillator is configured as a wearable cardiac defibrillator.

223. The cardiac defibrillator of claim 222, wherein the plurality of ECG electrodes, the plurality of therapy electrodes, and the defibrillation controller are configured to be worn by the patient.

224. The cardiac defibrillator of claim 223, further comprising a garment configured to be worn about a torso of the patient.
